(19) United States
(12) Patent Application Publication
(10) Pub. No.: US 2026/0201473 A1
(43) Pub. Date: Jul. 16, 2026

(54) METHODS AND COMPOSITIONS FOR PREDICTING AND TREATING TRIPLE NEGATIVE BREAST CANCER

(71) Applicants: CbsBioscience Co., Ltd., Daejeon (KR); NATIONAL CANCER CENTER, Goyang-si Gyeonggi-do (KR); KOREA UNIVERSITY RESEARCH AND BUSINESS FOUNDATION, Seoul (KR)

(72) Inventors: Jin Young Park, Daejeon (KR); Yunsuk Yu, Daejeon (KR); Chang Min Kim, Daejeon (KR); Sun-Young Kong, Goyang (KR); Kyong Hwa Park, Seoul (KR)

(21) Appl. No.: 19/127,778

(22) PCT Filed: Nov. 7, 2023
(86) PCT No.: PCT/IB2023/061238

§ 371 Date: May 7, 2025

Related U.S. Application Data
(60) Provisional application No. 63/382,702, filed on Nov. 7, 2022.

(51) Int. Cl. C12Q 1/6886; G01N 33/57515

(52) U.S. Cl. CPC C12Q 1/6886; G01N 33/57515; C12Q 2600/118; C12Q 2600/158

(57) ABSTRACT

Biomarkers that can be used for the detection or diagnosis of disease states, preferably cancer (e.g., triple negative breast cancer (TNBC)) disease states, to the prediction of disease prognosis and/or a treatment outcome, to the identification of a treatment regimen for cancer (e.g., TNBC), and/or to indicate the responsiveness to the treatment regimen for cancer (e.g., TNBC) in a subject are described. Also described are probes capable of detecting the biomarkers and related methods and kits for determining cancer (e.g., TNBC) disease states and/or identification of treatment regimens for the cancer (e.g., TNBC) disease states.

### CROSS REFERENCE TO RELATED APPLICATION

This application claims priority to U.S. Provisional Application No. 63/382,702, filed Nov. 7, 2022, the disclosure of which is herein incorporated by reference in its entirety.

### FIELD OF THE INVENTION

The present invention is directed generally to the detection or diagnosis of disease states, preferably cancer (e.g., triple negative breast cancer (TNBC)) disease states, to predict disease prognosis and/or a treatment outcome, to the identification of a treatment regimen for cancer (e.g., TNBC), and/or to indicate the responsiveness to the treatment regimen and/or surgical treatment for cancer (e.g., TNBC) in a subject. Further, the present invention provides a method, system, computer program, reagents, and/or kits useful for these purposes. Provided herein are a panel of biomarkers that are indicative of, diagnostic for, useful for predictions of disease prognosis and identification of a treatment regimen, and/or indicative of responsiveness to the treatment regimen and/or surgical treatment for cancer (e.g., TNBC) states, and probes capable of detecting the panel of biomarkers and related methods and kits thereof. Also provided herein are methods, systems, and computer programs for predicting disease prognosis, and/or response to treatment, of cancer (e.g., TNBC) in a subject.

### BACKGROUND OF THE INVENTION

Breast cancer is one of the most frequently diagnosed cancers and is increasing globally, including in Korea (1-3). Owing to recent research, the clinical application of new therapeutics, and precision medicine approaches, the prognosis of patients with breast cancer has greatly improved over time (4).

For better treatment outcomes in breast cancer, intensive research has enabled the development of a multidisciplinary approach utilizing the rational application of surgery, radiation, systemic chemotherapy, and endocrine therapy. Of note, escalation or de-escalation of adjuvant systemic treatment based on multi-gene assays has become a standard of care in patients with hormone receptor-positive cancers (5, 6). However, TNBC has still remained as a collective entity of most challenging clinical subtype of breast cancer with various biological features. Traditionally, it has been immunohistochemically identified as the lack of expression of the estrogen receptor (ER)/progesterone receptor (PR)/human epidermal growth factor receptor-2 (HER2) (ER/PR/HER2). Compared with other subtypes with hormone receptor or HER-2, prognosis of TNBC is poor due to its aggressive biology and high metastatic potential even after a good response to standard systemic chemotherapy (7). With the advancement of new technologies, TNBC has been categorized according to molecular characteristics in multi-omic analyses (8-11), most extensively studied using PAM-50 defined subtypes. However, none has been identified as a biomarker for predicting high-risk patients, guiding treatment, and discovery of new therapeutic targets. Thus, to establish a more elaborate therapeutic strategy in patients with this subtype, significant effort should be made to figure out the unmet need for biomarkers that accurately predict prognosis and response to treatment.

### SUMMARY OF THE INVENTION

In one general aspect, the invention relates to an isolated set of probes capable of detecting a panel of biomarkers comprising at least two biomarkers selected from the group consisting of ankyrin repeat domain 36 (ANKRD36), ankyrin repeat domain 36B pseudogene 2 (ANKRD36BP2), B-box and SPRY domain-containing (BSPRY), chromosome 12 open reading frame 65 (C12orf65), chromosome 2 open reading frame 49 (C2orf49), chromosome 1 open reading frame 198 (C1orf198), coiled-coil domain containing 114 (CCDC114), claudin 4 (CLDN4), CUE domain containing 1 (CUEDC1), outer dynein arm docking complex subunit 1 (ODAD1), cell growth regulator with EF-hand domain 1 (CGREF1), DEP domain containing 7 (DEPDC7), doublecortin like kinase 2 (DCLK2), diacylglycerol kinase (DGKH), disco interacting protein 2 homolog B (DIP2B), disrupted-in-schizophrenia 1 (DISC1), epithelial membrane protein 1 (EMP1), ERH mRNA splicing and mitosis factor (ERH), growth arrest and DNA damage inducible beta (GADD45B), glutaminase (GLS), grainyhead like transcription factor 1 (GRHL1), glycophorin C (GYPC), H2A histone family member X (H2AFX), HRas proto-oncogene (HRAS), intracellular adhesion molecule 1 (ICAM1), interphotoreceptor matrix proteoglycan 2 (IMPG2), potassium voltage-gated channel subfamily C member 3 (KCNC3), kruppel like factor 6 (KLF6), kruppel like factor 7 (KLF7), keratin 17 (KRT17), LON peptidase protein (LRBA), leucine rich repeat, Ig-like and transmembrane domains 3 (LRIT3), leucine rich repeat containing 37B (LRRC37B), LSM11, U7 small nuclear RNA associated (LSM11), lysosomal trafficking regulator (LYST), metastasis associated lung adenocarcinoma transcript 1 (MALAT1), minichromosome maintenance complex component 3 associated protein antisense RNA 1 (MCM3AP_AS1), MICAL like 2 (MICALL2), MHC class I polypeptide-related sequence B (MICB), metallothionein 2A (MT2A), myelin expression factor 2 (MYEF2), NEDD4 binding protein 3 (N4BP3), neuroblastoma breakpoint family member 20 (NBPF20), NADH: Ubiquinone Oxidoreductase Core Subunit V2 (NDUFV2), neurogenic locus notch homolog protein 2 (NOTCH2), NADPH oxidase activator 1 (NOXA1), natriuretic peptide receptor 3 (NPR3), nuclear receptor subfamily 6 group A member 1 (NR6A1), P21 (RAC1) activated kinase 3 (PAK3), pantothenate kinase 3 (PANK3), par-6 family cell polarity regulator beta (PARD6B), peroxisomal biogenesis factor 1 (PEX1), piggyBac transposable element derived 4 (PGBD4), praja ring finger ubiquitin ligase 1 (PJA1), pleckstrin homology and FYVE domain containing 1 (PLEKHF1), purine nucleoside phosphorylase (PNP), protein tyrosine phosphatase receptor type A (PTPRA), protein phosphatase, Mg2+/Mn2+ dependent 1K (PPM1K), prickle planar cell polarity protein 1 (PRICKLE1), protein kinase AMP-activated non-catalytic subunit beta 2 (PRKAB2), PYD and CARD domain containing (PYCARD), RAS p21 protein activator 1 (RASA1), RASD family member 2 (RASD2), RAS guanyl releasing protein 1 (RASGRP1), rhophilin RHO GTPase binding protein 2 (RHPN2), Rab interacting lysosomal protein like 2 (RILPL2), roundabout guidance receptor 1 (ROBO1), RAR related orphan receptor A (RORA), Stromal cell derived factor 4 (SDF4), SERTA domain containing 4 (SERTAD4), shisa family member 5 (SHISA5), signal induced proliferation associated 1 like 2 (SIPA1L2), solute carrier family 22 member 20 (SLC22A20P), solute carrier family 24 member 3 (SLC24A3), solute carrier family 2 member 12 (SLC2A12), solute carrier family 39 member 10 (SLC39A10), solute carrier family 25 member 40 (SLC25A40), solute carrier family 43 member 1 (SLC43A1), solute carrier family 45 member 4 (SLC45A4), solute carrier family 6 member 20 (SLC6A20), spectrin beta, erthrocytic (SPTB), stromal antigen 3-like 3 (STAG3L3), sushi domain containing 3 (SUSD3), TATA-Box binding protein associated factor 10 (TAF10), t-complex-associated testis expressed 3 (TCTE3), transmembrane channel like 7 (TMC7), transmembrane and coiled-coil domain family 2 (TMCC2), transcriptional repressor GATA binding 1 (TRPS1), tubulin tyrosine ligase like 4 (TTLL4), tRNA-yW synthesizing protein 5 (TYW5), ubiquitin conjugating enzyme E2 W (UBE2 W), WD repeat, sterile alpha motif and U-box domain containing 1 (WDSUB1), N-protein N-terminal glutamine amidohydrolase (WDYHV1), N-terminal glutamine amidase 1 (NTAQ1), zinc finger BED-type containing 6 (ZBED6), zinc finger and BTB domain containing 46 (ZBTB46), zinc finger CCCH-type containing 13 (ZC3H13), zinc fingers and homeoboxes 2 (ZHX2), zinc finger protein 217 (ZNF217), zinc finger protein 233 (ZNF233), zinc finger protein 248 (ZNF248), zinc finger protein 469 (ZNF469), and zinc finger protein 785 (ZNF785).

In certain embodiments, the panel of biomarkers comprises at least two biomarkers selected from the group consisting of DGKH, KLF7, NR6A1, PYCARD, ROBO1, SLC22A20P, SLC24A3, DIP2B, EMP1, NOTCH2, RORA, NOXA1, CUEDC1, PRICKLE1, DCKL2, C12orf65, GADD45B, LRBA, LYST, PEX1, PRKAB2, TYW5, ODAD1, DEPDC7, MICALL2, SLC43A1, SLC6A20, RASA1, SLC45A4, NTAQ1, CGREF1, MICB, LSM11, PJA1, C2orf49, HRAS, KCNC3, MT2A, LRIT3, SHISA5, SLC25A40, H2AFX, PTPRA, RILPL2, ZC3CH13, ZHX2, CDLN4, ERH, GYPC, MTA2, NDUFV2, SDF4, and UBE2 W.

In certain embodiments, the panel of biomarkers comprises at least three biomarkers, at least four biomarkers, at least five biomarkers, at least six biomarkers, at least seven biomarkers, at least eight biomarkers, at least nine biomarkers, or ten biomarkers selected from the biomarker signature as follows: (a) DGKH, GADD45B, KLF7, LYST, NR6A1, PYCARD, ROBO1, SLC22A20P, SLC24A3, and SLC45A4; (b) DGKH, DIP2B, EMP1, GADD45B, MT2A, NOTCH2, NR6A1, RORA, SLC22A20P, and SL24A3; (c) GADD45B, LYST, NOXA1, NR6A1, PYCARD, SLC22A20P, SLC24A3, and NTAQ1; (d) DGKH, KLF7, LYST, NR6A1, ROBO1, SLC24A3, and SLC6A20; (e) DGKH, EMP1, GADD45B, LYST, SLC22A20P, SLC24A3, and SLC6A20; (f) CUEDC1, DGKH, EMP1, LYST, NOXA1, SLC22A20P, and SLC6A20; (g) DGKH, KLF7, LYST, NR6A1, PRICKLE1, ROBO1, and SLC24A3; (h) DCLK2, GADD45B, LYST, NR6A1, SLC22A20P, SLC24A3, and NTAQ1; (i) GADD45B, KLF7, LYST, NR6A1, ROBO1, SLC22A20P, and SLC6A20; (j) DCLK2, GADD45B, LYST, NR6A1, SLC22A20P, and NTAQ1; (k) C12orf65, GADD45B, LRBA, LYST, PEX1, PRKAB2, and TYW5; (l) LYST, PEX1, ODAD1, DEPDC7, MICALL2, SLC43A1, and SLC6A20; (m) C12orf65, GADD45B, LYST, PEX1, RASA1, SLC45A4, and NTAQ1; (n) TYW5, DEPDC7, SLC43A1, CGREF1, MICB, HRAS, and MT2A; (o) LRBA, LYST, PEX1, DEPDC7, SLC43A1, MICB, and C2orf49; (p) GADD45B, PEX1, DEPDC7, SLC43A1, LSM11, and PJA1; (q) DEPDC7, SLC43A1, MICB, C2orf49, HRAS, KCNC3, and MT2A; (r) LYST, DEPDC7, SLC43A1, SLC6A20, MICB, and HRAS; (s) PEX1, MICALL2, SLC43A1, RASA1, KCNC3, and LRIT3; (t) GADD45B, SLC43A1, SLC45A4, KCNC3, SHISA5, and SLC25A40; (u) GADD45B, H2AFX, PTPRA, RILPL2, RORA, ZC3H13, and ZHX2; or (v) CLDN4, ERH, GADD45B, GYPC, H2AFX, MT2A, NDUFV2, SDF4, and UBE2 W.

In certain embodiments, the probe is selected from the group consisting of an aptamer, an antibody, an affibody, a peptide, a protein, an organic molecule, and a nucleic acid.

In certain embodiments, a computer-implemented method is provided for predicting prognosis, and/or response to treatment, of cancer (e.g., breast cancer, i.e., a TNBC), the computer-implemented method comprising: (a) receiving computer-readable data of a panel of biomarkers for a sample from a subject; (b) analyzing the computer-readable data; (c) generating a risk score based on the analysis; (d) predicting a prognosis of cancer (e.g., breast cancer, i.e., a TNBC) in the subject based on the risk score and/or analysis of the computer-readable data; and (e) classifying the subject as high risk or low risk for disease progression, relapse, recurrence, and/or death based on the risk score. In an embodiment, the analyzing the computer-readable data includes identifying a pattern of the panel of the biomarkers in the received computer-readable data that is predictive and/or determinative of a cancer (e.g., breast cancer, i.e., a TNBC) prognosis. In certain embodiments, the computer-readable data can include data for an isolated set of probes capable of detecting a panel of biomarkers, including, for example, at least two, at least three, at least four, at least five, at least six, at least seven, at least eight, at least nine, or ten biomarkers selected from the group ankyrin repeat domain 36 (ANKRD36), ankyrin repeat domain 36B pseudogene 2 (ANKRD36BP2), B-box and SPRY domain-containing (BSPRY), chromosome 12 open reading frame 65 (C12orf65), chromosome 2 open reading frame 49 (C2orf49), chromosome 1 open reading frame 198 (C1orf198), coiled-coil domain containing 114 (CCDC114), claudin 4 (CLDN4), CUE domain containing 1 (CUEDC1), outer dynein arm docking complex subunit 1 (ODAD1), cell growth regulator with EF-hand domain 1 (CGREF1), DEP domain containing 7 (DEPDC7), doublecortin like kinase 2 (DCLK2), diacylglycerol kinase (DGKH), disco interacting protein 2 homolog B (DIP2B), disrupted-in-schizophrenia 1 (DISC1), epithelial membrane protein 1 (EMP1), ERH mRNA splicing and mitosis factor (ERH), growth arrest and DNA damage inducible beta (GADD45B), glutaminase (GLS), grainyhead like transcription factor 1 (GRHL1), glycophorin C (GYPC), H2A histone family member X (H2AFX), HRas proto-oncogene (HRAS), intracellular adhesion molecule 1 (ICAM1), interphotoreceptor matrix proteoglycan 2 (IMPG2), potassium voltage-gated channel subfamily C member 3 (KCNC3), kruppel like factor 6 (KLF6), kruppel like factor 7 (KLF7), keratin 17 (KRT17), LON peptidase N-terminal domain and RING finger protein 2 (LONRF2), LPS responsive beige-like anchor protein (LRBA), leucine rich repeat, Ig-like and transmembrane domains 3 (LRIT3), leucine rich repeat containing 37B (LRRC37B), LSM11, U7 small nuclear RNA associated (LSM11), lysosomal trafficking regulator (LYST), metastasis associated lung adenocarcinoma transcript 1 (MALAT1), minichromosome maintenance complex component 3 associated protein antisense RNA 1 (MCM3AP_AS1), MICAL like 2 (MICALL2), MHC class I polypeptide-related sequence B (MICB), metallothionein 2A (MT2A), myelin expression factor 2 (MYEF2), NEDD4 binding protein 3 (N4BP3), neuroblastoma breakpoint family member 20 (NBPF20), NADH: Ubiquinone Oxidoreductase Core Subunit V2 (NDUFV2), neurogenic locus notch homolog protein 2 (NOTCH2), NADPH oxidase activator 1 (NOXA1), natriuretic peptide receptor 3 (NPR3), nuclear receptor subfamily 6 group A member 1 (NR6A1), P21 (RAC1) activated kinase 3 (PAK3), pantothenate kinase 3 (PANK3), par-6 family cell polarity regulator beta (PARD6B), peroxisomal biogenesis factor 1 (PEX1), piggyBac transposable element derived 4 (PGBD4), praja ring finger ubiquitin ligase 1 (PJA1), pleckstrin homology and FYVE domain containing 1 (PLEKHF1), purine nucleoside phosphorylase (PNP), protein tyrosine phosphatase receptor type A (PTPRA), protein phosphatase, Mg2+/Mn2+ dependent 1K (PPM1K), prickle planar cell polarity protein 1 (PRICKLE1), protein kinase AMP-activated non-catalytic subunit beta 2 (PRKAB2), PYD and CARD domain containing (PYCARD), RAS p21 protein activator 1 (RASA1), RASD family member 2 (RASD2), RAS guanyl releasing protein 1 (RASGRP1), rhophilin RHO GTPase binding protein 2 (RHPN2), Rab interacting lysosomal protein like 2 (RILPL2), roundabout guidance receptor 1 (ROBO1), RAR related orphan receptor A (RORA), Stromal cell derived factor 4 (SDF4), SERTA domain containing 4 (SERTAD4), shisa family member 5 (SHISA5), signal induced proliferation associated 1 like 2 (SIPA1L2), solute carrier family 22 member 20 (SLC22A20P), solute carrier family 24 member 3 (SLC24A3), solute carrier family 2 member 12 (SLC2A12), solute carrier family 39 member 10 (SLC39A10), solute carrier family 25 member 40 (SLC25A40), solute carrier family 43 member 1 (SLC43A1), solute carrier family 45 member 4 (SLC45A4), solute carrier family 6 member 20 (SLC6A20), spectrin beta, erthrocytic (SPTB), stromal antigen 3-like 3 (STAG3L3), sushi domain containing 3 (SUSD3), TATA-Box binding protein associated factor 10 (TAF10), t-complex-associated testis expressed 3 (TCTE3), transmembrane channel like 7 (TMC7), transmembrane and coiled-coil domain family 2 (TMCC2), transcriptional repressor GATA binding 1 (TRPS1), tubulin tyrosine ligase like 4 (TTLL4), tRNA-yW synthesizing protein 5 (TYW5), ubiquitin conjugating enzyme E2 W (UBE2 W), WD repeat, sterile alpha motif and U-box domain containing 1 (WDSUB1), N-protein N-terminal glutamine amidohydrolase (WDYHV1), N-terminal glutamine amidase 1 (NTAQ1), zinc finger BED-type containing 6 (ZBED6), zinc finger and BTB domain containing 46 (ZBTB46), zinc finger CCCH-type containing 13 (ZC3H13), zinc fingers and homeoboxes 2 (ZHX2), zinc finger protein 217 (ZNF217), zinc finger protein 233 (ZNF233), zinc finger protein 248 (ZNF248), zinc finger protein 469 (ZNF469), and zinc finger protein 785 (ZNF785). In certain embodiments, the method further comprises treating the cancer (e.g., breast cancer, i.e., TNBC) in the subject based on the classification of the subject.

In certain embodiments, a system is provided for predicting prognosis, and/or response to treatment, of cancer (e.g., breast cancer, i.e., TNBC), the system comprising: (a) a receiver configured to receive computer-readable data of a panel of biomarkers for a sample from a subject; and (b) a system configured to (i) analyze the computer-readable data, (ii) generate a risk score based on the analysis, (iii) predict a prognosis of TNBC in the subject based on the risk score and/or analysis of the computer-readable data, and (iv) classify the subject as high risk or low risk for disease progression, relapse, recurrence, and/or death based on the risk score. In certain embodiments, the computer-readable data can include data for an isolated set of probes capable of detecting a panel of biomarkers, including, for example, at least two, at least three, at least four, at least five, at least six, at least seven, at least eight, at least nine, or ten biomarkers selected from the group consisting of ANKRD36, ANKRD36BP2, BSPRY, C12orf65, C2orf49, C1orf198, CCDC114, CLDN4, CUEDC1, ODAD1, CGREF1, DEPDC7, DCLK2, DGKH, DIP2B, DISC1, EMP1, ERH, GADD45B, GLS, GRHL1, GYPC, H2AFX, HRAS, ICAM1, IMPG2, KCNC3, KLF6, KLF7, KRT17, LONRF2, LRBA, LRIT3, LRRC37B, LSM11, LYST, MALAT1, MCM3AP_AS1, MICALL2, MICB, MT2A, MYEF2, N4BP3, NBPF20, NDUFV2, NOTCH2, NOXA1, NPR3, NR6A1, PAK3, PANK3, PARD6B, PEX1, PGBD4, PJA1, PLEKHF1, PNP, PPM1K, PRICKLE1, PRKAB2, PTPRA, PYCARD, RASA1, RASD2, RASGRP1, RHPN2, RILPL2, ROBO1, RORA, SDF4, SERTAD4, SHISA5, SIPA1L2, SLC22A20P, SLC24A3, SLC2A12, SLC39A10, SLC25A40, SLC43A1, SLC45A4, SLC6A20, SPTB, STAG3L3, SUSD3, TAF10, TCTE3, TMC7, TMCC2, TRPS1, TTLL4, TYW5, UBE2 W, WDSUB1, WDYHV1, NTAQ1, ZBED6, ZBTB46, ZC3H13, ZHX2, ZNF217, ZNF233, ZNF248, ZNF469, and ZNF785. In an embodiment, the system is configured to analyze the computer-readable data and identify a pattern of the panel of the biomarkers in the received computer-readable data that is predictive and/or determinative of a cancer (e.g., TNBC) prognosis and/or treatment outcome. In certain embodiments, the system comprises formulating and outputting, via a display or other user interface device, a treatment regimen for treating the cancer (e.g., TNBC) in the subject based on the classification of the subject.

Also provided are methods for predicting a disease prognosis and/or a treatment outcome for a subject diagnosed with cancer (e.g., breast cancer, i.e., TNBC), the method comprising (a) obtaining a sample from the subject; (b) contacting the sample with the isolated set of probes to detect a panel of biomarkers in the sample, wherein the panel of biomarkers comprises at least two biomarkers selected from the group consisting of ANKRD36, ANKRD36BP2, BSPRY, C12orf65, C2orf49, C1orf198, CCDC114, CLDN4, CUEDC1, ODAD1, CGREF1, DEPDC7, DCLK2, DGKH, DIP2B, DISC1, EMP1, ERH, GADD45B, GLS, GRHL1, GYPC, H2AFX, HRAS, ICAM1, IMPG2, KCNC3, KLF6, KLF7, KRT17, LONRF2, LRBA, LRIT3, LRRC37B, LSM11, LYST, MALAT1, MCM3AP_AS1, MICALL2, MICB, MT2A, MYEF2, N4BP3, NBPF20, NDUFV2, NOTCH2, NOXA1, NPR3, NR6A1, PAK3, PANK3, PARD6B, PEX1, PGBD4, PJA1, PLEKHF1, PNP, PPM1K, PRICKLE1, PRKAB2, PTPRA, PYCARD, RASA1, RASD2, RASGRP1, RHPN2, RILPL2, ROBO1, RORA, SDF4, SERTAD4, SHISA5, SIPA1L2, SLC22A20P, SLC24A3, SLC2A12, SLC39A10, SLC25A40, SLC43A1, SLC45A4, SLC6A20, SPTB, STAG3L3, SUSD3, TAF10, TCTE3, TMC7, TMCC2, TRPS1, TTLL4, TYW5, UBE2 W, WDSUB1, WDYHV1, NTAQ1, ZBED6, ZBTB46, ZC3H13, ZHX2, ZNF217, ZNF233, ZNF248, ZNF469, and ZNF785; (c) analyzing a pattern of the panel of biomarkers to determine a risk score for the subject. In certain embodiments, the methods further comprise (d) classifying the subject as high risk or low risk for disease progression, relapse, recurrence, and/or death based on the risk score. In certain embodiments, the method further comprises treating the cancer (e.g., TNBC) in the subject based on the classification of the subject.

In certain embodiments, the panel of biomarkers comprises the biomarkers of a biomarker signature as follows: (a) DGKH, GADD45B, KLF7, LYST, NR6A1, PYCARD, ROBO1, SLC22A20P, SLC24A3, and SLC45A4; (b) DGKH, DIP2B, EMP1, GADD45B, MT2A, NOTCH2, NR6A1, RORA, SLC22A20P, and SL24A3; (c) GADD45B, LYST, NOXA1, NR6A1, PYCARD, SLC22A20P, SLC24A3, and NTAQ1; (d) DGKH, KLF7, LYST, NR6A1, ROBO1, SLC24A3, and SLC6A20; (e) DGKH, EMP1, GADD45B, LYST, SLC22A20P, SLC24A3, and SLC6A20; (f) CUEDC1, DGKH, EMP1, LYST, NOXA1, SLC22A20P, and SLC6A20; (g) DGKH, KLF7, LYST, NR6A1, PRICKLE1, ROBO1, and SLC24A3; (h) DCLK2, GADD45B, LYST, NR6A1, SLC22A20P, SLC24A3, and NTAQ1; (i) GADD45B, KLF7, LYST, NR6A1, ROBO1, SLC22A20P, and SLC6A20; (j) DCLK2, GADD45B, LYST, NR6A1, SLC22A20P, and NTAQ1; (k) C12orf65, GADD45B, LRBA, LYST, PEX1, PRKAB2, and TYW5; (l) LYST, PEX1, ODAD1, DEPDC7, MICALL2, SLC43 A1, and SLC6A20; (m) C12orf65, GADD45B, LYST, PEX1, RASA1, SLC45A4, and NTAQ1; (n) TYW5, DEPDC7, SLC43A1, CGREF1, MICB, HRAS, and MT2A; (o) LRBA, LYST, PEX1, DEPDC7, SLC43A1, MICB, and C2orf49; (p) GADD45B, PEX1, DEPDC7, SLC43A1, LSM11, and PJA1; (q) DEPDC7, SLC43A1, MICB, C2orf49, HRAS, KCNC3, and MT2A; (r) LYST, DEPDC7, SLC43A1, SLC6A20, MICB, and HRAS; (s) PEX1, MICALL2, SLC43A1, RASA1, KCNC3, and LRIT3; (t) GADD45B, SLC43A1, SLC45A4, KCNC3, SHISA5, and SLC25A40; (u) GADD45B, H2AF, PTPRA, RILPL2, RORA, ZC3H13, and ZHX2; or (v) CLDN4, ERH, GADD45B, GYPC, H2AFX, MT2A, NDUFV2, SDF4, and UBE2 W.

In certain embodiments, the sample is a tissue sample, a blood sample, or a urine sample. The tissue sample can, for example, be a fresh frozen tumor tissue sample or a fixed formalin paraffin embedded tumor tissue sample.

In certain embodiments, the subject is high risk for disease progression, relapse, recurrence, and/or death, and the method further comprises administering an advanced, strengthened, or standard treatment to the subject to treat the cancer (e.g., TNBC). The advanced, strengthened, or standard treatment can, for example, in the case of early-stage TNBC, comprise surgery and administering a chemotherapeutic agent, radiotherapy, an immunotherapeutic agent, any novel treatment, or a combination of those treatments as a neoadjuvant treatment, an adjuvant treatment, and/or a maintenance treatment. The chemotherapeutic agent can, for example, be selected from capecitabine, doxorubicin, cyclophosphamide, docetaxel, olaparib, carboplatin, paclitaxel, epirubicin, methotrexate, and/or fluorouracil. The immunotherapeutic agent can, for example, be an immune checkpoint inhibitor. The immune checkpoint inhibitor can be selected from pembrolizumab, atezolizumab, nivolumab, ipilimumab, durvalumab, and/or avelumab.

In certain embodiments, the subject is low risk for disease progression, relapse, recurrence, and/or death, and the method further comprises administering the standard or attenuated treatment for cancer (e.g., TNBC). The standard or attenuated treatment can, for example, in the case of early-stage TNBC, comprise surgery only or surgery and administering a chemotherapeutic agent, radiotherapy, an immunotherapeutic agent, any novel treatment, or a combination of those treatments as a neoadjuvant treatment, an adjuvant treatment, and/or a maintenance treatment. The chemotherapeutic agent can, for example, be selected from capecitabine, doxorubicin, cyclophosphamide, docetaxel, olaparib, carboplatin, paclitaxel, epirubicin, methotrexate, and/or fluorouracil. The immunotherapeutic agent can, for example, be an immune checkpoint inhibitor. The immune checkpoint inhibitor can be selected from pembrolizumab, atezolizumab, nivolumab, ipilimumab, durvalumab, and/or avelumab.

Also provided are kits for predicting disease prognosis and/or a treatment outcome for a subject diagnosed with TNBC, the kits comprising (a) an isolated set of probes capable of detecting a panel of biomarkers comprising at least two biomarkers selected from the group consisting of ankyrin repeat domain 36 (ANKRD36), ankyrin repeat domain 36B pseudogene 2 (ANKRD36BP2), B-box and SPRY domain-containing (BSPRY), chromosome 12 open reading frame 65 (C12orf65), chromosome 2 open reading frame 49 (C2orf49), chromosome 1 open reading frame 198 (C1orf198), coiled-coil domain containing 114 (CCDC114), claudin 4 (CLDN4), CUE domain containing 1 (CUEDC1), outer dynein arm docking complex subunit 1 (ODAD1), cell growth regulator with EF-hand domain 1 (CGREF1), DEP domain containing 7 (DEPDC7), doublecortin like kinase 2 (DCLK2), diacylglycerol kinase (DGKH), disco interacting protein 2 homolog B (DIP2B), disrupted-in-schizophrenia 1 (DISC1), epithelial membrane protein 1 (EMP1), ERH mRNA splicing and mitosis factor (ERH), growth arrest and DNA damage inducible beta (GADD45B), glutaminase (GLS), grainyhead like transcription factor 1 (GRHL1), glycophorin C (GYPC), H2A histone family member X (H2AFX), HRas proto-oncogene (HRAS), intracellular adhesion molecule 1 (ICAM1), interphotoreceptor matrix proteoglycan 2 (IMPG2), potassium voltage-gated channel subfamily C member 3 (KCNC3), kruppel like factor 6 (KLF6), kruppel like factor 7 (KLF7), keratin 17 (KRT17), LON peptidase protein (LRBA), leucine rich repeat, Ig-like and transmembrane domains 3 (LRIT3), leucine rich repeat containing 37B (LRRC37B), LSM11, U7 small nuclear RNA associated (LSM11), lysosomal trafficking regulator (LYST), metastasis associated lung adenocarcinoma transcript 1 (MALAT1), minichromosome maintenance complex component 3 associated protein antisense RNA 1 (MCM3AP_AS1), MICAL like 2 (MICALL2), MHC class I polypeptide-related sequence B (MICB), metallothionein 2A (MT2A), myelin expression factor 2 (MYEF2), NEDD4 binding protein 3 (N4BP3), neuroblastoma breakpoint family member 20 (NBPF20), NADH: Ubiquinone Oxidoreductase Core Subunit V2 (NDUFV2), neurogenic locus notch homolog protein 2 (NOTCH2), NADPH oxidase activator 1 (NOXA1), natriuretic peptide receptor 3 (NPR3), nuclear receptor subfamily 6 group A member 1 (NR6A1), P21 (RAC1) activated kinase 3 (PAK3), pantothenate kinase 3 (PANK3), par-6 family cell polarity regulator beta (PARD6B), peroxisomal biogenesis factor 1 (PEX1), piggyBac transposable element derived 4 (PGBD4), praja ring finger ubiquitin ligase 1 (PJA1), pleckstrin homology and FYVE domain containing 1 (PLEKHF1), purine nucleoside phosphorylase (PNP), protein tyrosine phosphatase receptor type A (PTPRA), protein phosphatase, Mg2+/Mn2+ dependent 1K (PPM1K), prickle planar cell polarity protein 1 (PRICKLE1), protein kinase AMP-activated non-catalytic subunit beta 2 (PRKAB2), PYD and CARD domain containing (PYCARD), RAS p21 protein activator 1 (RASA1), RASD family member 2 (RASD2), RAS guanyl releasing protein 1 (RASGRP1), rhophilin RHO GTPase binding protein 2 (RHPN2), Rab interacting lysosomal protein like 2 (RILPL2), roundabout guidance receptor 1 (ROBO1), RAR related orphan receptor A (RORA), Stromal cell derived factor 4 (SDF4), SERTA domain containing 4 (SERTAD4), shisa family member 5 (SHISA5), signal induced proliferation associated 1 like 2 (SIPA1L2), solute carrier family 22 member 20 (SLC22A20P), solute carrier family 24 member 3 (SLC24A3), solute carrier family 2 member 12 (SLC2A12), solute carrier family 39 member 10 (SLC39A10), solute carrier family 25 member 40 (SLC25A40), solute carrier family 43 member 1 (SLC43A1), solute carrier family 45 member 4 (SLC45A4), solute carrier family 6 member 20 (SLC6A20), spectrin beta, erthrocytic (SPTB), stromal antigen 3-like 3 (STAG3L3), sushi domain containing 3 (SUSD3), TATA-Box binding protein associated factor 10 (TAF10), t-complex-associated testis expressed 3 (TCTE3), transmembrane channel like 7 (TMC7), transmembrane and coiled-coil domain family 2 (TMCC2), transcriptional repressor GATA binding 1 (TRPS1), tubulin tyrosine ligase like 4 (TTLL4), tRNA-yW synthesizing protein 5 (TYW5), ubiquitin conjugating enzyme E2 W (UBE2 W), WD repeat, sterile alpha motif and U-box domain containing 1 (WDSUB1), N-protein N-terminal glutamine amidohydrolase (WDYHV1), N-terminal glutamine amidase 1 (NTAQ1), zinc finger BED-type containing 6 (ZBED6), zinc finger and BTB domain containing 46 (ZBTB46), zinc finger CCCH-type containing 13 (ZC3H13), zinc fingers and homeoboxes 2 (ZHX2), zinc finger protein 217 (ZNF217), zinc finger protein 233 (ZNF233), zinc finger protein 248 (ZNF248), zinc finger protein 469 (ZNF469), and zinc finger protein 785 (ZNF785); and (b) instructions for use.

In certain embodiments, the isolated set of probes capable of detecting a panel of biomarkers comprises at least three, at least four, at least five, at least six, at least seven, at least eight, at least nine, or ten biomarkers selected from the biomarker signature as follows: (a) DGKH, GADD45B, KLF7, LYST, NR6A1, PYCARD, ROBO1, SLC22A20P, SLC24A3, and SLC45A4; (b) DGKH, DIP2B, EMP1, GADD45B, MT2A, NOTCH2, NR6A1, RORA, SLC22A20P, and SL24A3; (c) GADD45B, LYST, NOXA1, NR6A1, PYCARD, SLC22A20P, SLC24A3, and NTAQ1; (d) DGKH, KLF7, LYST, NR6A1, ROBO1, SLC24A3, and SLC6A20; (e) DGKH, EMP1, GADD45B, LYST, SLC22A20P, SLC24A3, and SLC6A20; (f) CUEDC1, DGKH, EMP1, LYST, NOXA1, SLC22A20P, and SLC6A20; (g) DGKH, KLF7, LYST, NR6A1, PRICKLE1, ROBO1, and SLC24A3; (h) DCLK2, GADD45B, LYST, NR6A1, SLC22A20P, SLC24A3, and NTAQ1; (i) GADD45B, KLF7, LYST, NR6A1, ROBO1, SLC22A20P, and SLC6A20; (j) DCLK2, GADD45B, LYST, NR6A1, SLC22A20P, and NTAQ1; (k) C12orf65, GADD45B, LRBA, LYST, PEX1, PRKAB2, and TYW5; (l) LYST, PEX1, ODAD1, DEPDC7, MICALL2, SLC43A1, and SLC6A20; (m) C12orf65, GADD45B, LYST, PEX1, RASA1, SLC45A4, and NTAQ1; (n) TYW5, DEPDC7, SLC43A1, CGREF1, MICB, HRAS, and MT2A; (o) LRBA, LYST, PEX1, DEPDC7, SLC43A1, MICB, and C2orf49; (p) GADD45B, PEX1, DEPDC7, SLC43A1, LSM11, and PJA1; (q) DEPDC7, SLC43A1, MICB, C2orf49, HRAS, KCNC3, and MT2A; (r) LYST, DEPDC7, SLC43A1, SLC6A20, MICB, and HRAS; (s) PEX1, MICALL2, SLC43A1, RASA1, KCNC3, and LRIT3; (t) GADD45B, SLC43A1, SLC45A4, KCNC3, SHISA5, and SLC25A40; (u) GADD45B, H2AFX, PTPRA, RILPL2, RORA, ZC3H13, and ZHX2; or (v) CLDN4, ERH, GADD45B, GYPC, H2AFX, MT2A, NDUFV2, SDF4, and UBE2 W.

The probe can, for example, be selected from the group consisting of an aptamer, an antibody, an affibody, a protein, an organic molecule, and a nucleic acid.

Further aspects, features and advantages of the present invention will be better appreciated upon a reading of the following detailed description of the invention and claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1B. Selected prognostic gene signature evaluation of clinical performance. The clinical performance of the prognostic gene signature was evaluated using receiver operating characteristic (ROC) analysis, cross validation, and logistic regression analysis. (FIG. 1A) ROC analysis of the prognostic gene signature to predict the recurrence of TNBC. (FIG. 1B) Clinical performance of the gene signature in logistic regression analysis, cross-validation, and ROC analysis.

FIGS. 2A-2C. Invasive disease-free survival in high risk and low risk groups. The invasive disease-free survival was analyzed in different cases. (FIG. 2A) Kaplan-Meier curves for all patients. (FIG. 2B) Kaplan-Meier curves of patients treated with adjuvant chemotherapy (FIG. 2C) Kaplan-Meier curves of patients treated with neoadjuvant chemotherapy.

FIGS. 3A-3E. Prognostic validation of the gene signature in the validation cohort. The prognostic gene signature was validated by invasive disease-free survival analysis in various cases. (FIG. 3A) Kaplan-Meier curves for all patients. (FIG. 3B) Kaplan-Meier curves in surgical specimens (primary tumor of adjuvant patients and residual tumor of neoadjuvant patients). (FIG. 3C) Kaplan-Meier curves of patients treated with adjuvant chemotherapy (FIG. 3D) Kaplan-Meier curves in biopsies of patients treated with neoadjuvant chemotherapy. (FIG. 3E) Kaplan-Meier curves for invasive disease-free survival in residual tumors of patients treated with neoadjuvant chemotherapy.

FIG. 4. Flow chart of biomarker development. To develop biomarkers for the detection or diagnosis of a TNBC disease state, to predict a treatment outcome, to identify a treatment regimen for TNBC, and/or to indicate responsiveness to the treatment regimen for TNBC in a subject, in a first step, RNA sequencing is performed on a sample of interest to provide expression data for correlation analysis. From the analysis, differentially expressed genes are identified and a candidate gene signature is produced. The candidate gene signature is verified through cross validation, uni-/multi-variable analysis, and meta-analysis to produce a specific gene signature, which is then validated in a separate cohort of samples.

FIGS. 5A-5G. PAM50 Call Analysis. (FIG. 5A) PCA analysis of TNBC patients with PAM50 Call. (FIG. 5B) Kaplan-Meier curves for basal subtype. (FIG. 5C) Kaplan-Meier curves for Her-2 subtype. (FIG. 5D) Kaplan-Meier curves for LumA subtype. (FIG. 5E) Kaplan-Meier curves for LumB subtype. (FIG. 5F) Kaplan-Meier curves for Normal subtype. (FIG. 5G) Kaplan-Meier curves for PAM50 Call ROR-S.

FIGS. 6A-6C. T-cell receptor repertoire analysis. (FIG. 6A) T-cell receptor beta diversity index in TNBC patients according to presence of recurrence. (FIG. 6B) ROC analysis of TRB diversity to predict recurrence of TNBC. (FIG. 6C) Kaplan-Meier curves with TRB diversity.

FIG. 7. Non-limiting embodiment of a system constructed according to the principles of the invention.

FIG. 8. Non-limiting embodiment of a computer-implemented process, according to the principles of the invention.

FIGS. 9A-9B. Selected prognostic gene signature evaluation of clinical performance. The clinical performance of the prognostic gene signature was evaluated using receiver operating characteristic (ROC) analysis, cross validation, and logistic regression analysis. (FIG. 9A) ROC analysis of the prognostic gene signature to predict the recurrence of TNBC. (FIG. 9B) Clinical performance of the gene signature in logistic regression analysis, cross-validation, and ROC analysis.

FIGS. 10A-10C. Invasive disease-free survival in high risk and low risk groups. The invasive disease-free survival was analyzed in different cases. (FIG. 10A) Kaplan-Meier curves for all patients. (FIG. 10B) Kaplan-Meier curves of patients treated with adjuvant chemotherapy (FIG. 10C) Kaplan-Meier curves of patients treated with neoadjuvant chemotherapy.

FIGS. 11A-11E. Prognostic validation of the gene signature in the validation cohort. The prognostic gene signature was validated by invasive disease-free survival analysis in various cases. (FIG. 11A) Kaplan-Meier curves for all patients. (FIG. 11B) Kaplan-Meier curves in surgical specimens (primary tumor of adjuvant patients and residual tumor of neoadjuvant patients). (FIG. 11C) Kaplan-Meier curves of patients treated with adjuvant chemotherapy (FIG. 11D) Kaplan-Meier curves in biopsies of patients treated with neoadjuvant chemotherapy. (FIG. 11E) Kaplan-Meier curves for invasive disease-free survival in residual tumors of patients treated with neoadjuvant chemotherapy.

FIGS. 12A-12G. PAM50 Call Analysis. (FIG. 12A) PCA analysis of TNBC patients with PAM50 Call. (FIG. 12B) Kaplan-Meier curves for basal subtype. (FIG. 12C) Kaplan-Meier curves for Her-2 subtype. (FIG. 12D) Kaplan-Meier curves for LumA subtype. (FIG. 12E) Kaplan-Meier curves for LumB subtype. (FIG. 12F) Kaplan-Meier curves for Normal subtype. (FIG. 12G) Kaplan-Meier curves for PAM50 Call ROR-S.

FIGS. 13A-13C. T-cell receptor repertoire analysis. (FIG. 13A) T-cell receptor beta diversity index in TNBC patients according to presence of recurrence. (FIG. 13B) ROC analysis of TRB diversity to predict recurrence of TNBC. (FIG. 13C) Kaplan-Meier curves with TRB diversity.

FIGS. 14A-14C. Immune Cell Clustering. The same type of immune cells clustered closely (FIG. 14A). Further sub-cluster was analyzed in each t cell cluster and myeloid cluster of prior clustering (FIG. 14B, 14C).

FIGS. 15A-15E. Gene-set Signatures related to CD8+ T cells. Gene set signature related to CD8+ T cells were identified as following: GADD45B, H2AFX, PTPRA, TILPL2, RORA, ZC3H13, ZHX2 (AUC=0.927, sensitivity=92.31%, specificity=93.65%, accuracy=93.42%). (FIG. 15A, 15B) In Kaplan Meier (KM) analysis, patients with tumors with high-risk gene signatures (n=16, median iDFS=42.7 months) showed significantly shorter iDFS than those with low-risk gene signatures (n=60, median iDFS not reached). (FIG. 15C, 15D) CD8+ T cells related gene signature was marked on CD8+ T cell near the CD4+ T cell in t-SNE (FIG. 15E).

FIGS. 16A-16E. Gene-set Signatures related to Macrophages. Gene-set signatures related to macrophages were identified as following; CLDN4, ERH, GADD45B, GYPC, H2AFX, MT2A, NDUFV2, SDF4, UBE2 W (AUC=0.963, sensitivity=92.31%; specificity=93.65%; accuracy=93.42%) (FIG. 16A, 16B). In Kaplan Meier (KM) analysis, patients with tumors with high-risk gene signatures (n=16, median iDFS=42.7 months) showed significantly shorter iDFS than those with low-risk gene signatures (n=60, median iDFS not reached) (FIG. 16C, 16D). Macrophages related gene signature was marked on macrophage, monocyte and dendritic cells in t-SNE (FIG. 16E).

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, a system, an article, or an apparatus that comprises a list of elements is not necessarily limited to only those elements but can include other elements not expressly listed or inherent to such composition, mixture, process, method, article, system, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, “biomarker” refers to a gene or protein whose level of expression or concentration in a sample is altered compared to that of a normal or healthy sample or is indicative of a condition. The biomarkers disclosed herein are genes and/or proteins whose expression level or concentration or timing of expression or concentration correlates with the capability of determining whether a subject has a high risk or low risk for a cancer (e.g., breast cancer, i.e., TNBC) disease state. As used herein, the term “high risk” means that there is a high risk of developing the cancer, a high risk of further progression of the cancer, a high risk of relapse or recurrence of cancer, and/or a high risk of death. As used herein, the term “low risk” means that there is a low risk of developing the cancer, a low risk of further progression of the cancer, a low risk of relapse or recurrence of the cancer, and/or a low risk of death. Determining whether a subject is high risk or low risk for a cancer (e.g., breast cancer, i.e., TNBC) disease state can, for example, result in determining a treatment outcome for a biological therapy in a subject diagnosed with cancer (e.g., breast cancer, i.e., TNBC) and/or determining a biological therapy treatment program for a subject with cancer (e.g., breast cancer, i.e., TNBC).

As used herein, “probe” refers to any molecule or agent that is capable of selectively binding to an intended target biomolecule. The target molecule can be a biomarker, for example, a nucleotide transcript or a protein encoded by or corresponding to a biomarker. Probes can be synthesized by one of skill in the art, or derived from appropriate biological preparations, in view of the present disclosure. Probes can be specifically designed to be labeled. Examples of molecules that can be utilized as probes include, but are not limited to, RNA, DNA, proteins, peptides, antibodies, aptamers, affibodies, and organic molecules.

As used herein, “predicting a treatment outcome” when referring to a subject with cancer (e.g., breast cancer, such as a TNBC) means that the panel of biomarkers can, for example, determine and/or be determinative of which subjects will be responsive to which specific treatment for the cancer (e.g., the breast cancer, i.e., the TNBC). By way of an example, the methods disclosed herein can predict and/or determine if a subject is high risk for further progression of TNBC, and, if a subject is predicted and/or determined to be high risk, the subject can be treated with an advanced, strengthened, or standard form of treatment comprising, for example, in the case of early-stage TNBC, surgery and further chemotherapeutic agents, radiotherapy, immunotherapeutic agents, any novel therapeutics, or combination of those treatments as a neoadjuvant treatment, an adjuvant treatment, and/or a maintenance treatment. By way of another example, the methods disclosed herein can predict and/or determine if a subject is low risk for further progression of TNBC, and, if the subject is predicted and/or determined to be low risk, the subject can be treated with the standard or attenuated treatment comprising, for example, in the case of early-stage TNBC, surgery only or surgery and chemotherapeutic agents, radiotherapy, immunotherapeutic agents, any novel therapeutics, or combination of those treatments as a neoadjuvant treatment, an adjuvant treatment, and/or maintenance treatment.

As used herein, “prognosis of triple negative breast cancer” or “prognosis of TNBC” refers to anticipating various conditions of the subject suffering from TNBC, such as, for example, full recovery from TNBC, possibility of recurrence of TNBC, and/or possibility of survival of subject after being diagnosed with TNBC. This can vary based on various conditions, such as, for example, severity of the TNBC, diagnosis point, and/or treatment progress. TNBC can be treated efficiently when various treatment methods are properly applied according to the prognosis.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

As used herein, “sample” is intended to include any sampling of cells, tissues, or bodily fluids in which expression of a biomarker can be detected. Examples of such samples include, but are not limited to, biopsies, smears, blood, lymph, urine, saliva, or any other bodily secretion or derivative thereof. Blood can, for example, include whole blood, plasma, serum, or any derivative of blood. Samples can be obtained from a subject by a variety of techniques, which are known to those skilled in the art. The sample can, for example, be a fresh frozen tumor sample. The sample can, for example, be formalin fixed paraffin embedded (FFPE) tumor tissue sample.

The term “administering” with respect to the methods of the invention, means a method for therapeutically or prophylactically preventing, treating or ameliorating a syndrome, disorder or disease (e.g., cancer, such as breast cancer, i.e., TNBC) as described herein. Such methods include administering an effective amount of said therapeutic agent (e.g., a chemotherapy) at different times during the course of a therapy or concurrently in a combination form. The methods of the invention are to be understood as embracing all known therapeutic treatment regimens.

The term “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating a syndrome, disorder, or disease being treated, or the symptoms of a syndrome, disorder or disease being treated (e.g., cancer, such as breast cancer, i.e., TNBC).

### Biomarker Panel and Probes for Detecting the Biomarkers

The present invention relates generally to the prediction of a prognosis and/or treatment outcome for a treatment regimen for cancer (e.g., breast cancer, i.e., TNBC) in a subject, and provides methods, reagents, systems, and kits useful for this purpose. Provided herein are biomarkers that are predictive for prognosis of, and/or responsiveness to a treatment regimen for, cancer (e.g., breast cancer, such as a TNBC) in a subject. In certain embodiments, the present invention provides a panel of biomarkers (e.g., genes that are expressed or proteins in a subject at a specific time point) that can be used to predict and/or determine a prognosis of, and/or predict and/or determine a treatment regimen or indicate the responsiveness to the treatment regimen for, cancer (e.g., breast cancer, such as a TNBC).

Any methods available in the art for detecting expression of biomarkers are encompassed herein. The expression, presence, or amount of a biomarker of the invention can be detected on a nucleic acid level (e.g., as an RNA transcript) or a protein level. By “detecting or determining expression of a biomarker” is intended to include determining the quantity or presence of a protein or its RNA transcript for the biomarkers disclosed herein. Thus, “detecting expression” encompasses instances where a biomarker is determined not to be expressed, not to be detectably expressed, expressed at a low level, expressed at a normal level, or overexpressed.

In certain embodiments, provided herein are DNA-, RNA-, and protein-based diagnostic methods that either directly or indirectly detect the biomarkers described herein. The present invention also provides compositions, reagents, systems, and kits for such diagnostic purposes. The diagnostic methods described herein may be qualitative or quantitative. Quantitative diagnostic methods may be used, for example, to compare a detected biomarker level to a cutoff or threshold level. Where applicable, qualitative or quantitative diagnostic methods can also include amplification of target, signal, or intermediary.

In certain embodiments, biomarkers are detected at the nucleic acid (e.g., RNA) level. For example, the amount of biomarker RNA (e.g., mRNA) present in a sample is determined (e.g., to determine the level of biomarker expression). Biomarker nucleic acid (e.g., RNA, amplified cDNA, etc.) can be detected/quantified using a variety of nucleic acid techniques known to those of ordinary skill in the art, including but not limited to, nucleic acid hybridization and nucleic acid amplification, e.g., RNA-Seq (RNA Sequencing), a reverse transcription (RT)-polymerase chain reaction (PCR), RT-quantitative PCR (RT-qPCR), competitive RT-PCR, RNase protection assay, northern blot, and DNA chip.

In certain embodiments, a microarray is used to detect the biomarker. Microarrays can, for example, include DNA microarrays; protein microarrays; tissue microarrays; cell microarrays; chemical compound microarrays; and antibody microarrays. A DNA microarray, commonly referred to as a gene chip can be used to monitor expression levels of thousands of genes simultaneously. Microarrays can be used to identify disease genes by comparing expression in disease states versus normal states. Microarrays can also be used for diagnostic purposes, i.e., patterns of expression levels of genes can be studied in samples prior to the diagnosis of disease or after the diagnosis of disease (e.g., TNBC), and these patterns can later be used to predict prognosis of, and/or the treatment regimen for, a disease in a subject at risk of or diagnosed with a disease or the responsiveness to a particular treatment regimen for a disease in a subject at risk of or diagnosed with a disease.

In certain embodiments, the expression products are proteins corresponding to the biomarkers of the panel. In certain embodiments detecting the levels of expression products comprises exposing the sample to antibodies for the proteins corresponding to the biomarkers of the panel. In certain embodiments, the antibodies are covalently linked to a solid surface. In certain embodiments, detecting the levels of expression products comprises exposing the sample to a mass analysis technique (e.g., mass spectrometry).

Methods of detecting protein expression levels and/or patterns using antibodies include, but are not limited to western blot, ELISA (enzyme linked immunosorbent assay), radioimmunoassay, radioimmunodiffusion, Ouchterlony immunodiffusion analysis, rocket immunoelectrophoresis, immunohistochemistry, immunoprecipitation assay, complement fixation assay, fluorescent activated cell sorter (FACS), and protein chip.

In certain embodiments, reagents are provided for the detection and/or quantification of biomarker proteins. The reagents can include, but are not limited to, primary antibodies that bind the protein biomarkers, secondary antibodies that bind the primary antibodies, affibodies that bind the protein biomarkers, aptamers that bind the protein or nucleic acid biomarkers (e.g., RNA or DNA), and/or nucleic acids that bind the nucleic acid biomarkers (e.g., RNA or DNA). The detection reagents can be labeled (e.g., fluorescently) or unlabeled. Additionally, the detection reagents can be free in solution or immobilized.

In certain embodiments, when quantifying the level of a biomarker(s) present in a sample, the level can be determined on an absolute basis or a relative basis. When determined on an absolute or relative basis, comparisons can be made to controls, which can include, but are not limited to historical samples from the same patient (e.g., a series of samples over a certain time period), level(s) found in a subject or population of subjects without the disease or disorder (e.g., TNBC), a threshold value, and an acceptable range.

Thus, provided herein are isolated sets of probes capable of detecting a panel of biomarkers, which are indicative of predicting a treatment outcome for a subject with TNBC. In certain embodiments, provided is an isolated set of probes capable of detecting a panel of biomarkers comprising at least two biomarkers selected from the group consisting of ankyrin repeat domain 36 (ANKRD36), ankyrin repeat domain 36B pseudogene 2 (ANKRD36BP2), B-box and SPRY domain-containing (BSPRY), chromosome 12 open reading frame 65 (C12orf65), chromosome 2 open reading frame 49 (C2orf49), chromosome 1 open reading frame 198 (C1orf198), coiled-coil domain containing 114 (CCDC114), claudin 4 (CLDN4), CUE domain containing 1 (CUEDC1), outer dynein arm docking complex subunit 1 (ODAD1), cell growth regulator with EF-hand domain 1 (CGREF1), DEP domain containing 7 (DEPDC7), doublecortin like kinase 2 (DCLK2), diacylglycerol kinase (DGKH), disco interacting protein 2 homolog B (DIP2B), disrupted-in-schizophrenia 1 (DISC1), epithelial membrane protein 1 (EMP1), ERH mRNA splicing and mitosis factor (ERH), growth arrest and DNA damage inducible beta (GADD45B), glutaminase (GLS), grainyhead like transcription factor 1 (GRHL1), glycophorin C (GYPC), H2A histone family member X (H2AFX), HRas proto-oncogene (HRAS), intracellular adhesion molecule 1 (ICAM1), interphotoreceptor matrix proteoglycan 2 (IMPG2), potassium voltage-gated channel subfamily C member 3 (KCNC3), kruppel like factor 6 (KLF6), kruppel like factor 7 (KLF7), keratin 17 (KRT17), LON peptidase N-terminal domain and RING finger protein 2 (LONRF2), LPS responsive beige-like anchor protein (LRBA), leucine rich repeat, Ig-like and transmembrane domains 3 (LRIT3), leucine rich repeat containing 37B (LRRC37B), LSM11, U7 small nuclear RNA associated (LSM11), lysosomal trafficking regulator (LYST), metastasis associated lung adenocarcinoma transcript 1 (MALAT1), minichromosome maintenance complex component 3 associated protein antisense RNA 1 (MCM3AP_AS1), MICAL like 2 (MICALL2), MHC class I polypeptide-related sequence B (MICB), metallothionein 2A (MT2A), myelin expression factor 2 (MYEF2), NEDD4 binding protein 3 (N4BP3), neuroblastoma breakpoint family member 20 (NBPF20), NADH: Ubiquinone Oxidoreductase Core Subunit V2 (NDUFV2), neurogenic locus notch homolog protein 2 (NOTCH2), NADPH oxidase activator 1 (NOXA1), natriuretic peptide receptor 3 (NPR3), nuclear receptor subfamily 6 group A member 1 (NR6A1), P21 (RAC1) activated kinase 3 (PAK3), pantothenate kinase 3 (PANK3), par-6 family cell polarity regulator beta (PARD6B), peroxisomal biogenesis factor 1 (PEX1), piggyBac transposable element derived 4 (PGBD4), praja ring finger ubiquitin ligase 1 (PJA1), pleckstrin homology and FYVE domain containing 1 (PLEKHF1), purine nucleoside phosphorylase (PNP), protein tyrosine phosphatase receptor type A (PTPRA), protein phosphatase, Mg2+/Mn2+ dependent 1K (PPM1K), prickle planar cell polarity protein 1 (PRICKLE1), protein kinase AMP-activated non-catalytic subunit beta 2 (PRKAB2), PYD and CARD domain containing (PYCARD), RAS p21 protein activator 1 (RASA1), RASD family member 2 (RASD2), RAS guanyl releasing protein 1 (RASGRP1), rhophilin RHO GTPase binding protein 2 (RHPN2), Rab interacting lysosomal protein like 2 (RILPL2), roundabout guidance receptor 1 (ROBO1), RAR related orphan receptor A (RORA), Stromal cell derived factor 4 (SDF4), SERTA domain containing 4 (SERTAD4), shisa family member 5 (SHISA5), signal induced proliferation associated 1 like 2 (SIPA1L2), solute carrier family 22 member 20 (SLC22A20P), solute carrier family 24 member 3 (SLC24A3), solute carrier family 2 member 12 (SLC2A12), solute carrier family 39 member 10 (SLC39A10), solute carrier family 25 member 40 (SLC25A40), solute carrier family 43 member 1 (SLC43A1), solute carrier family 45 member 4 (SLC45A4), solute carrier family 6 member 20 (SLC6A20), spectrin beta, erthrocytic (SPTB), stromal antigen 3-like 3 (STAG3L3), sushi domain containing 3 (SUSD3), TATA-Box binding protein associated factor 10 (TAF10), t-complex-associated testis expressed 3 (TCTE3), transmembrane channel like 7 (TMC7), transmembrane and coiled-coil domain family 2 (TMCC2), transcriptional repressor GATA binding 1 (TRPS1), tubulin tyrosine ligase like 4 (TTLL4), tRNA-yW synthesizing protein 5 (TYW5), ubiquitin conjugating enzyme E2 W (UBE2 W), WD repeat, sterile alpha motif and U-box domain containing 1 (WDSUB1), N-protein N-terminal glutamine amidohydrolase (WDYHV1), N-terminal glutamine amidase 1 (NTAQ1), zinc finger BED-type containing 6 (ZBED6), zinc finger and BTB domain containing 46 (ZBTB46), zinc finger CCCH-type containing 13 (ZC3H13), zinc fingers and homeoboxes 2 (ZHX2), zinc finger protein 217 (ZNF217), zinc finger protein 233 (ZNF233), zinc finger protein 248 (ZNF248), zinc finger protein 469 (ZNF469), and zinc finger protein 785 (ZNF785).

In certain embodiments, the panel of biomarkers comprises at least two biomarkers selected from the group consisting of DGKH, KLF7, NR6A1, PYCARD, ROBO1, SLC22A20P, SLC24A3, DIP2B, EMP1, NOTCH2, RORA, NOXA1, CUEDC1, PRICKLE1, DCKL2, C12orf65, GADD45B, LRBA, LYST, PEX1, PRKAB2, TYW5, ODAD1, DEPDC7, MICALL2, SLC43A1, SLC6A20, RASA1, SLC45A4, NTAQ1, CGREF1, MICB, LSM11, PJA1, C2orf49, HRAS, KCNC3, MT2A, LRIT3, SHISA5, SLC25A40, H2AFX, PTPRA, RILPL2, ZC3CH13, ZHX2, CDLN4, ERH, GYPC, MTA2, NDUFV2, SDF4, and UBE2 W.

In certain embodiments, the isolated set of probes is capable of detecting a panel of biomarkers comprising 3 or more, 4 or more, 5 or more, 6 or more, 7 or more, 8 or more, 9 or more, or 10 biomarkers.

In certain embodiments, the panel of biomarkers comprises at least two biomarkers, at least three biomarkers, at least four biomarkers, at least five biomarkers, at least six biomarkers, at least seven biomarkers, at least eight biomarkers, at least nine biomarkers, or ten biomarkers selected from a biomarker signature as follows: (a) DGKH, GADD45B, KLF7, LYST, NR6A1, PYCARD, ROBO1, SLC22A20P, SLC24A3, and SLC45A4; (b) DGKH, DIP2B, EMP1, GADD45B, MT2A, NOTCH2, NR6A1, RORA, SLC22A20P, and SL24A3; (c) GADD45B, LYST, NOXA1, NR6A1, PYCARD, SLC22A20P, SLC24A3, and NTAQ1; (d) DGKH, KLF7, LYST, NR6A1, ROBO1, SLC24A3, and SLC6A20; (e) DGKH, EMP1, GADD45B, LYST, SLC22A20P, SLC24A3, and SLC6A20; (f) CUEDC1, DGKH, EMP1, LYST, NOXA1, SLC22A20P, and SLC6A20; (g) DGKH, KLF7, LYST, NR6A1, PRICKLE1, ROBO1, and SLC24A3; (h) DCLK2, GADD45B, LYST, NR6A1, SLC22A20P, SLC24A3, and NTAQ1; (i) GADD45B, KLF7, LYST, NR6A1, ROBO1, SLC22A20P, and SLC6A20; (j) DCLK2, GADD45B, LYST, NR6A1, SLC22A20P, and NTAQ1; (k) C12orf65, GADD45B, LRBA, LYST, PEX1, PRKAB2, and TYW5; (1) LYST, PEX1, ODAD1, DEPDC7, MICALL2, SLC43A1, and SLC6A20; (m) C12orf65, GADD45B, LYST, PEX1, RASA1, SLC45A4, and NTAQ1; (n) TYW5, DEPDC7, SLC43A1, CGREF1, MICB, HRAS, and MT2A; (o) LRBA, LYST, PEX1, DEPDC7, SLC43A1, MICB, and C2orf49; (p) GADD45B, PEX1, DEPDC7, SLC43A1, LSM11, and PJA1; (q) DEPDC7, SLC43A1, MICB, C2orf49, HRAS, KCNC3, and MT2A; (r) LYST, DEPDC7, SLC43A1, SLC6A20, MICB, and HRAS; (s) PEX1, MICALL2, SLC43A1, RASA1, KCNC3, and LRIT3; (t) GADD45B, SLC43A1, SLC45A4, KCNC3, SHISA5, and SLC25A40; (u) GADD45B, H2AFX, PTPRA, RILPL2, RORA, ZC3H13, and ZHX2; or (v) CLDN4, ERH, GADD45B, GYPC, H2AFX, MT2A, NDUFV2, SDF4, and UBE2 W.

In certain embodiments, the panel of biomarkers comprises the biomarkers of (a) diacylglycerol kinase (DGKH), growth arrest and DNA damage inducible beta (GADD45B), kruppel like factor (KLF7), lysosomal trafficking regulator (LYST), nuclear receptor subfamily 6 group A member 1 (NR6A1), PYD and CARD domain containing (PYCARD), roundabout guidance receptor 1 (ROBO1), solute carrier family 22 member 20 pseudogene (SLC22A20P), solute carrier family 24 member 3 (SLC24A3), and solute carrier family 45 member 4 (SLC45A4); (b) chromosome 12 open reading frame 65 (C12orf65), growth arrest and DNA damage inducible beta (GADD45B), LPS responsive beige-like anchor protein (LRBA), lysosomal trafficking regulator (LYST), peroxisomal biogenesis factor 1 (PEX1), protein kinase AMP-activated non-catalytic subunit beta 2 (PRKAB2), and tRNA-yW synthesizing protein 5 (TYW5); (c) growth arrest and DNA damage inducible beta (GADD45B), H2A histone family member X (H2AFX), protein tyrosine phosphatase receptor type A (PTPRA), Rab interacting lysosomal protein like 2 (RILPL2), RAR related orphan receptor A (RORA), zinc finger CCCH-type containing 13 (ZC3H13), and zinc fingers and homeoboxes 2 (ZHX2); or (d) claudin 4 (CLDN4), ERH mRNA splicing and mitosis factor (ERH), growth arrest and DNA damage inducible beta (GADD45B), glycophorin C (GYPC), H2A histone family member X (H2AFX), metallothionein 2A (MT2A), NADH: Ubiquinone Oxidoreductase Core Subunit V2 (NDUFV2), Stromal cell derived factor 4 (SDF4), and ubiquitin conjugating enzyme E2 W (UBE2 W).

The probe can be any molecule or agent that specifically detects a biomarker. In certain embodiments, the probe is selected from the group consisting of an aptamer, an antibody, an affibody, a peptide, and a nucleic acid (such as an oligonucleotide hybridizing to the gene or mRNA of a biomarker). An aptamer is an oligonucleotide or a peptide that binds specifically to a target molecule. An aptamer is usually created by selection from a large random sequence pool. Examples of aptamers useful for the invention include oligonucleotides, such as DNA, RNA or nucleic acid analogues, or peptides, that bind to a biomarker of the invention.

### Methods of Use

Provided are methods of predicting a prognosis of, and/or a treatment outcome for, a subject with cancer or an at risk-candidate of developing cancer. The methods comprise (a) obtaining a sample from the subject; (b) contacting the sample with the isolated set of probes capable of detecting a panel of biomarkers in the sample; and (c) analyzing a pattern of the panel of biomarkers to determine a risk score for the subject. In certain embodiments, the methods further comprise (d) classifying the subject as high risk or low risk for disease progression, relapse, recurrence, and/or death based on the risk score. In certain embodiments, the cancer is breast cancer. In certain embodiments, the breast cancer is a triple negative breast cancer (TNBC). In certain embodiments, the TNBC is an early stage TNBC. In certain embodiments, the method further comprises treating the TNBC in the subject based on the classification of the subject.

In certain embodiments, the subject is classified as high risk, and the method may be advanced, strengthened, or standard form of treatment comprising, for example, in the case of early-stage TNBC, surgery and/or chemotherapeutic agents, radiotherapy, immunotherapeutic agents, any novel therapeutics, or a combination of those treatments as a neoadjuvant treatment, an adjuvant treatment, and/or a maintenance treatment. The chemotherapeutic agent can, for example, comprise capecitabine, doxorubicin, cyclophosphamide, docetaxel, olaparib, carboplatin, paclitaxel, epirubicin, methotrexate, and/or fluorouracil. The immunotherapeutic agent can, for example, be an immune checkpoint inhibitor. The immune checkpoint inhibitor can, for example, comprise pembrolizumab, atezolizumab, nivolumab, ipilimumab, durvalumab, and/or avelumab.

In certain embodiments, the subject is classified as low risk, and the method may be the standard or attenuated treatment comprising, for example, in the case of early-stage TNBC, surgery only or surgery and chemotherapeutic agents, radiotherapy, immunotherapeutic agents, any novel therapeutics, or a combination of those treatments as a neoadjuvant treatment, an adjuvant treatment, and/or maintenance treatment. The chemotherapeutic agent can, for example, comprise capecitabine, doxorubicin, cyclophosphamide, docetaxel, olaparib, carboplatin, paclitaxel, epirubicin, methotrexate, and/or fluorouracil. The immunotherapeutic agent can, for example, be an immune checkpoint inhibitor. The immune checkpoint inhibitor can, for example, comprise pembrolizumab, atezolizumab, nivolumab, ipilimumab, durvalumab, and/or avelumab.

The sample can, for example, be a tissue sample, a blood sample or urine sample. Preferably, the sample is a tissue sample from the subject. The tissue sample, can, for example, be a fixed formalin paraffin embedded tumor tissue sample.

In certain embodiments, the panel of biomarkers comprises a biomarker signature as follows: (a) DGKH, GADD45B, KLF7, LYST, NR6A1, PYCARD, ROBO1, SLC22A20P, SLC24A3, and SLC45A4; (b) DGKH, DIP2B, EMP1, GADD45B, MT2A, NOTCH2, NR6A1, RORA, SLC22A20P, and SL24A3; (c) GADD45B, LYST, NOXA1, NR6A1, PYCARD, SLC22A20P, SLC24A3, and NTAQ1; (d) DGKH, KLF7, LYST, NR6A1, ROBO1, SLC24A3, and SLC6A20; (e) DGKH, EMP1, GADD45B, LYST, SLC22A20P, SLC24A3, and SLC6A20; (f) CUEDC1, DGKH, EMP1, LYST, NOXA1, SLC22A20P, and SLC6A20; (g) DGKH, KLF7, LYST, NR6A1, PRICKLE1, ROBO1, and SLC24A3; (h) DCLK2, GADD45B, LYST, NR6A1, SLC22A20P, SLC24A3, and NTAQ1; (i) GADD45B, KLF7, LYST, NR6A1, ROBO1, SLC22A20P, and SLC6A20; (j) DCLK2, GADD45B, LYST, NR6A1, SLC22A20P, and NTAQ1; (k) C12orf65, GADD45B, LRBA, LYST, PEX1, PRKAB2, and TYW5; (1) LYST, PEX1, ODAD1, DEPDC7, MICALL2, SLC43A1, and SLC6A20; (m) C12orf65, GADD45B, LYST, PEX1, RASA1, SLC45A4, and NTAQ1; (n) TYW5, DEPDC7, SLC43A1, CGREF1, MICB, HRAS, and MT2A; (o) LRBA, LYST, PEX1, DEPDC7, SLC43A1, MICB, and C2orf49; (p) GADD45B, PEX1, DEPDC7, SLC43A1, LSM11, and PJA1; (q) DEPDC7, SLC43A1, MICB, C2orf49, HRAS, KCNC3, and MT2A; (r) LYST, DEPDC7, SLC43A1, SLC6A20, MICB, and HRAS; (s) PEX1, MICALL2, SLC43A1, RASA1, KCNC3, and LRIT3; (t) GADD45B, SLC43A1, SLC45A4, KCNC3, SHISA5, and SLC25A40; (u) GADD45B, H2AFX, PTPRA, RILPL2, RORA, ZC3H13, and ZHX2; or (v) CLDN4, ERH, GADD45B, GYPC, H2AFX, MT2A, NDUFV2, SDF4, and UBE2 W.

### Kits

Also provided are kits for predicting a response to a treatment regimen for a cancer (e.g., a breast cancer, such as a TNBC) in a subject. The kits can, for example, comprise (a) an isolated set of probes capable of detecting a panel of biomarkers comprising at least two, at least three, at least four, at least five, at least six, at least seven, at least eight, at least nine, or ten biomarkers selected from a biomarker signature as follows: (a) DGKH, GADD45B, KLF7, LYST, NR6A1, PYCARD, ROBO1, SLC22A20P, SLC24A3, and SLC45A4; (b) DGKH, DIP2B, EMP1, GADD45B, MT2A, NOTCH2, NR6A1, RORA, SLC22A20P, and SL24A3; (c) GADD45B, LYST, NOXA1, NR6A1, PYCARD, SLC22A20P, SLC24A3, and NTAQ1; (d) DGKH, KLF7, LYST, NR6A1, ROBO1, SLC24A3, and SLC6A20; (e) DGKH, EMP1, GADD45B, LYST, SLC22A20P, SLC24A3, and SLC6A20; (f) CUEDC1, DGKH, EMP1, LYST, NOXA1, SLC22A20P, and SLC6A20; (g) DGKH, KLF7, LYST, NR6A1, PRICKLE1, ROBO1, and SLC24A3; (h) DCLK2, GADD45B, LYST, NR6A1, SLC22A20P, SLC24A3, and NTAQ1; (i) GADD45B, KLF7, LYST, NR6A1, ROBO1, SLC22A20P, and SLC6A20; (j) DCLK2, GADD45B, LYST, NR6A1, SLC22A20P, and NTAQ1; (k) C12orf65, GADD45B, LRBA, LYST, PEX1, PRKAB2, and TYW5; (l) LYST, PEX1, ODAD1, DEPDC7, MICALL2, SLC43A1, and SLC6A20; (m) C12orf65, GADD45B, LYST, PEX1, RASA1, SLC45A4, and NTAQ1; (n) TYW5, DEPDC7, SLC43A1, CGREF1, MICB, HRAS, and MT2A; (o) LRBA, LYST, PEX1, DEPDC7, SLC43A1, MICB, and C2orf49; (p) GADD45B, PEX1, DEPDC7, SLC43A1, LSM11, and PJA1; (q) DEPDC7, SLC43A1, MICB, C2orf49, HRAS, KCNC3, and MT2A; (r) LYST, DEPDC7, SLC43A1, SLC6A20, MICB, and HRAS; (s) PEX1, MICALL2, SLC43A1, RASA1, KCNC3, and LRIT3; (t) GADD45B, SLC43A1, SLC45A4, KCNC3, SHISA5, and SLC25A40; (u) GADD45B, H2AFX, PTPRA, RILPL2, RORA, ZC3H13, and ZHX2; or (v) CLDN4, ERH, GADD45B, GYPC, H2AFX, MT2A, NDUFV2, SDF4, and UBE2 W; and (b) instructions for use.

In certain embodiments, the isolated set of probes capable of detecting a panel of biomarkers comprises the biomarker signature of (a) diacylglycerol kinase (DGKH), growth arrest and DNA damage inducible beta (GADD45B), kruppel like factor (KLF7), lysosomal trafficking regulator (LYST), nuclear receptor subfamily 6 group A member 1 (NR6A1), PYD and CARD domain containing (PYCARD), roundabout guidance receptor 1 (ROBO1), solute carrier family 22 member 20 pseudogene (SLC22A20P), solute carrier family 24 member 3 (SLC24A3), and solute carrier family 45 member 4 (SLC45A4); (b) chromosome 12 open reading frame 65 (C12orf65), growth arrest and DNA damage inducible beta (GADD45B), LPS responsive beige-like anchor protein (LRBA), lysosomal trafficking regulator (LYST), peroxisomal biogenesis factor 1 (PEX1), protein kinase AMP-activated non-catalytic subunit beta 2 (PRKAB2), and tRNA-yW synthesizing protein 5 (TYW5); (c) growth arrest and DNA damage inducible beta (GADD45B), H2A histone family member X (H2AFX), protein tyrosine phosphatase receptor type A (PTPRA), Rab interacting lysosomal protein like 2 (RILPL2), RAR related orphan receptor A (RORA), zinc finger CCCH-type containing 13 (ZC3H13), and zinc fingers and homeoboxes 2 (ZHX2); or (d) claudin 4 (CLDN4), ERH mRNA splicing and mitosis factor (ERH), growth arrest and DNA damage inducible beta (GADD45B), glycophorin C (GYPC), H2A histone family member X (H2AFX), metallothionein 2A (MT2A), NADH: Ubiquinone Oxidoreductase Core Subunit V2 (NDUFV2), Stromal cell derived factor 4 (SDF4), and ubiquitin conjugating enzyme E2 W (UBE2 W).

Compositions for use in the methods disclosed herein include, but are not limited to, probes, antibodies, affibodies, nucleic acids, and/or aptamers. Preferred compositions can detect the level of expression (e.g., mRNA or protein level) of a panel of biomarkers from a biological sample.

Any of the compositions can be provided in the form of a kit or a reagent mixture. By way of an example, labeled probes can be provided in a kit for the detection of a panel of biomarkers. Kits can include all components necessary or sufficient for assays, which can include, but is not limited to, detection reagents (e.g., probes), buffers, control reagents (e.g., positive and negative controls), amplification reagents, solid supports, labels, instruction manuals, etc. In certain embodiments, the kit comprises a set of probes for the panel of biomarkers and a solid support to immobilize the set of probes. In certain embodiments, the kit comprises a set of probes for the panel of biomarkers, a solid support, and reagents for processing the sample to be tested (e.g., reagents to isolate the protein or nucleic acids from the sample).

### Computer-Implemented Method

Provided are computer-implemented methods of predicting a prognosis of, and/or a treatment outcome for, a subject diagnosed with cancer (e.g., breast cancer, such as a TNBC), or an at risk-candidate of developing cancer (e.g., breast cancer, such as a TNBC). The computer-implemented methods comprise (a) receiving computer-readable data of a panel of biomarkers for a sample from a subject; (b) generating a risk score based on the analysis; (c) predicting a prognosis of TNBC in the subject based on the risk score and/or analysis of the computer-readable data; and (d) classifying the subject as high risk or low risk for disease progression, relapse, recurrence, and/or death based on the risk score. In certain embodiments, the computer-readable data can include data of an isolated set of probes capable of detecting a panel of biomarkers, including, for example, at least two, at least three, at least four, at least five, at least six, at least seven, at least eight, at least nine, or ten biomarkers selected from a biomarker signature as follows: (a) DGKH, GADD45B, KLF7, LYST, NR6A1, PYCARD, ROBO1, SLC22A20P, SLC24A3, and SLC45A4; (b) DGKH, DIP2B, EMP1, GADD45B, MT2A, NOTCH2, NR6A1, RORA, SLC22A20P, and SL24A3; (c) GADD45B, LYST, NOXA1, NR6A1, PYCARD, SLC22A20P, SLC24A3, and NTAQ1; (d) DGKH, KLF7, LYST, NR6A1, ROBO1, SLC24A3, and SLC6A20; (e) DGKH, EMP1, GADD45B, LYST, SLC22A20P, SLC24A3, and SLC6A20; (f) CUEDC1, DGKH, EMP1, LYST, NOXA1, SLC22A20P, and SLC6A20; (g) DGKH, KLF7, LYST, NR6A1, PRICKLE1, ROBO1, and SLC24A3; (h) DCLK2, GADD45B, LYST, NR6A1, SLC22A20P, SLC24A3, and NTAQ1; (i) GADD45B, KLF7, LYST, NR6A1, ROBO1, SLC22A20P, and SLC6A20; (j) DCLK2, GADD45B, LYST, NR6A1, SLC22A20P, and NTAQ1; (k) C12orf65, GADD45B, LRBA, LYST, PEX1, PRKAB2, and TYW5; (l) LYST, PEX1, ODAD1, DEPDC7, MICALL2, SLC43A1, and SLC6A20; (m) C12orf65, GADD45B, LYST, PEX1, RASA1, SLC45A4, and NTAQ1; (n) TYW5, DEPDC7, SLC43A1, CGREF1, MICB, HRAS, and MT2A; (o) LRBA, LYST, PEX1, DEPDC7, SLC43A1, MICB, and C2orf49; (p) GADD45B, PEX1, DEPDC7, SLC43A1, LSM11, and PJA1; (q) DEPDC7, SLC43A1, MICB, C2orf49, HRAS, KCNC3, and MT2A; (r) LYST, DEPDC7, SLC43A1, SLC6A20, MICB, and HRAS; (s) PEX1, MICALL2, SLC43A1, RASA1, KCNC3, and LRIT3; (t) GADD45B, SLC43A1, SLC45A4, KCNC3, SHISA5, and SLC25A40; (u) GADD45B, H2AFX, PTPRA, RILPL2, RORA, ZC3H13, and ZHX2; or (v) CLDN4, ERH, GADD45B, GYPC, H2AFX, MT2A, NDUFV2, SDF4, and UBE2 W. In an embodiment, the analyzing the computer-readable data includes identifying a pattern of the panel of the biomarkers in the received computer-readable data that is predictive and/or determinative of a cancer (e.g., breast cancer, such as a TNBC) prognosis. In certain embodiments, the method further comprises treating the cancer (e.g., breast cancer, such as a TNBC) in the subject based on the classification of the subject.

### System

Provided are systems for predicting a prognosis of, and/or a treatment outcome for, a subject diagnosed with cancer (e.g., breast cancer, such as a TNBC), or an at risk-candidate of developing cancer (e.g., a breast cancer, such as a TNBC). The systems comprise (a) a receiver configured to receive computer-readable data of a panel of biomarkers for a sample from a subject; and (b) a system configured to (i) analyze the computer-readable data, (ii) generate a risk score based on the analysis, (iii) predict a prognosis of cancer (e.g., breast cancer, such as a TNBC) in the subject based on the risk score and/or analysis of the computer-readable data, and (iv) classify the subject as high risk or low risk for disease progression, relapse, recurrence, and/or death based on the risk score. In certain embodiments, the computer-readable data can include data of an isolated set of probes capable of detecting a panel of biomarkers, including, for example, at least two, at least three, at least four, at least five, at least six, at least seven, at least eight, at least nine, or ten biomarkers selected from a biomarker signature as follows: (a) DGKH, GADD45B, KLF7, LYST, NR6A1, PYCARD, ROBO1, SLC22A20P, SLC24A3, and SLC45A4; (b) DGKH, DIP2B, EMP1, GADD45B, MT2A, NOTCH2, NR6A1, RORA, SLC22A20P, and SL24A3; (c) GADD45B, LYST, NOXA1, NR6A1, PYCARD, SLC22A20P, SLC24A3, and NTAQ1; (d) DGKH, KLF7, LYST, NR6A1, ROBO1, SLC24A3, and SLC6A20; (e) DGKH, EMP1, GADD45B, LYST, SLC22A20P, SLC24A3, and SLC6A20; (f) CUEDC1, DGKH, EMP1, LYST, NOXA1, SLC22A20P, and SLC6A20; (g) DGKH, KLF7, LYST, NR6A1, PRICKLE1, ROBO1, and SLC24A3; (h) DCLK2, GADD45B, LYST, NR6A1, SLC22A20P, SLC24A3, and NTAQ1; (i) GADD45B, KLF7, LYST, NR6A1, ROBO1, SLC22A20P, and SLC6A20; (j) DCLK2, GADD45B, LYST, NR6A1, SLC22A20P, and NTAQ1; (k) C12orf65, GADD45B, LRBA, LYST, PEX1, PRKAB2, and TYW5; (l) LYST, PEX1, ODAD1, DEPDC7, MICALL2, SLC43A1, and SLC6A20; (m) C12orf65, GADD45B, LYST, PEX1, RASA1, SLC45A4, and NTAQ1; (n) TYW5, DEPDC7, SLC43A1, CGREF1, MICB, HRAS, and MT2A; (o) LRBA, LYST, PEX1, DEPDC7, SLC43A1, MICB, and C2orf49; (p) GADD45B, PEX1, DEPDC7, SLC43A1, LSM11, and PJA1; (q) DEPDC7, SLC43A1, MICB, C2orf49, HRAS, KCNC3, and MT2A; (r) LYST, DEPDC7, SLC43A1, SLC6A20, MICB, and HRAS; (s) PEX1, MICALL2, SLC43A1, RASA1, KCNC3, and LRIT3; (t) GADD45B, SLC43A1, SLC45A4, KCNC3, SHISA5, and SLC25A40; (u) GADD45B, H2AFX, PTPRA, RILPL2, RORA, ZC3H13, and ZHX2; or (v) CLDN4, ERH, GADD45B, GYPC, H2AFX, MT2A, NDUFV2, SDF4, and UBE2 W. In certain embodiments, the system comprises formulating and outputting, via a display or other user interface device, a treatment regimen for treating the cancer (e.g., breast cancer, such as a TNBC) in the subject based on the classification of the subject.

### EMBODIMENTS

The invention provides also the following non-limiting embodiments.

Embodiment 1 is an isolated set of probes capable of detecting a panel of biomarkers comprising at least two biomarkers selected from the group consisting of ankyrin repeat domain 36 (ANKRD36), ankyrin repeat domain 36B pseudogene 2 (ANKRD36BP2), B-box and SPRY domain-containing (BSPRY), chromosome 12 open reading frame 65 (C12orf65), chromosome 2 open reading frame 49 (C2orf49), chromosome 1 open reading frame 198 (C1orf198), coiled-coil domain containing 114 (CCDC114), claudin 4 (CLDN4), CUE domain containing 1 (CUEDC1), outer dynein arm docking complex subunit 1 (ODAD1), cell growth regulator with EF-hand domain 1 (CGREF1), DEP domain containing 7 (DEPDC7), doublecortin like kinase 2 (DCLK2), diacylglycerol kinase (DGKH), disco interacting protein 2 homolog B (DIP2B), disrupted-in-schizophrenia 1 (DISC1), epithelial membrane protein 1 (EMP1), ERH mRNA splicing and mitosis factor (ERH), growth arrest and DNA damage inducible beta (GADD45B), glutaminase (GLS), grainyhead like transcription factor 1 (GRHL1), glycophorin C (GYPC), H2A histone family member X (H2AFX), HRas proto-oncogene (HRAS), intracellular adhesion molecule 1 (ICAM1), interphotoreceptor matrix proteoglycan 2 (IMPG2), potassium voltage-gated channel subfamily C member 3 (KCNC3), kruppel like factor 6 (KLF6), kruppel like factor 7 (KLF7), keratin 17 (KRT17), LON peptidase N-terminal domain and RING finger protein 2 (LONRF2), LPS responsive beige-like anchor protein (LRBA), leucine rich repeat, Ig-like and transmembrane domains 3 (LRIT3), leucine rich repeat containing 37B (LRRC37B), LSM11, U7 small nuclear RNA associated (LSM11), lysosomal trafficking regulator (LYST), metastasis associated lung adenocarcinoma transcript 1 (MALAT1), minichromosome maintenance complex component 3 associated protein antisense RNA 1 (MCM3AP_AS1), MICAL like 2 (MICALL2), MHC class I polypeptide-related sequence B (MICB), metallothionein 2A (MT2A), myelin expression factor 2 (MYEF2), NEDD4 binding protein 3 (N4BP3), neuroblastoma breakpoint family member 20 (NBPF20), NADH: Ubiquinone Oxidoreductase Core Subunit V2 (NDUFV2), neurogenic locus notch homolog protein 2 (NOTCH2), NADPH oxidase activator 1 (NOXA1), natriuretic peptide receptor 3 (NPR3), nuclear receptor subfamily 6 group A member 1 (NR6A1), P21 (RAC1) activated kinase 3 (PAK3), pantothenate kinase 3 (PANK3), par-6 family cell polarity regulator beta (PARD6B), peroxisomal biogenesis factor 1 (PEX1), piggyBac transposable element derived 4 (PGBD4), praja ring finger ubiquitin ligase 1 (PJA1), pleckstrin homology and FYVE domain containing 1 (PLEKHF1), purine nucleoside phosphorylase (PNP), protein tyrosine phosphatase receptor type A (PTPRA), protein phosphatase, Mg2+/Mn2+ dependent 1K (PPM1K), prickle planar cell polarity protein 1 (PRICKLE1), protein kinase AMP-activated non-catalytic subunit beta 2 (PRKAB2), PYD and CARD domain containing (PYCARD), RAS p21 protein activator 1 (RASA1), RASD family member 2 (RASD2), RAS guanyl releasing protein 1 (RASGRP1), rhophilin RHO GTPase binding protein 2 (RHPN2), Rab interacting lysosomal protein like 2 (RILPL2), roundabout guidance receptor 1 (ROBO1), RAR related orphan receptor A (RORA), Stromal cell derived factor 4 (SDF4), SERTA domain containing 4 (SERTAD4), shisa family member 5 (SHISA5), signal induced proliferation associated 1 like 2 (SIPA1L2), solute carrier family 22 member 20 (SLC22A20P), solute carrier family 24 member 3 (SLC24A3), solute carrier family 2 member 12 (SLC2A12), solute carrier family 39 member 10 (SLC39A10), solute carrier family 25 member 40 (SLC25A40), solute carrier family 43 member 1 (SLC43A1), solute carrier family 45 member 4 (SLC45A4), solute carrier family 6 member 20 (SLC6A20), spectrin beta, erthrocytic (SPTB), stromal antigen 3-like 3 (STAG3L3), sushi domain containing 3 (SUSD3), TATA-Box binding protein associated factor 10 (TAF10), t-complex-associated testis expressed 3 (TCTE3), transmembrane channel like 7 (TMC7), transmembrane and coiled-coil domain family 2 (TMCC2), transcriptional repressor GATA binding 1 (TRPS1), tubulin tyrosine ligase like 4 (TTLL4), tRNA-yW synthesizing protein 5 (TYW5), ubiquitin conjugating enzyme E2 W (UBE2 W), WD repeat, sterile alpha motif and U-box domain containing 1 (WDSUB1), N-protein N-terminal glutamine amidohydrolase (WDYHV1), N-terminal glutamine amidase 1 (NTAQ1), zinc finger BED-type containing 6 (ZBED6), zinc finger and BTB domain containing 46 (ZBTB46), zinc finger CCCH-type containing 13 (ZC3H13), zinc fingers and homeoboxes 2 (ZHX2), zinc finger protein 217 (ZNF217), zinc finger protein 233 (ZNF233), zinc finger protein 248 (ZNF248), zinc finger protein 469 (ZNF469), and zinc finger protein 785 (ZNF785).

Embodiment 2 is the isolated set of probes of embodiment 1, wherein the panel of biomarkers comprises at least two biomarkers selected from the group consisting of DGKH, KLF7, NR6A1, PYCARD, ROBO1, SLC22A20P, SLC24A3, DIP2B, EMP1, NOTCH2, RORA, NOXA1, CUEDC1, PRICKLE1, DCKL2, C12orf65, GADD45B, LRBA, LYST, PEX1, PRKAB2, TYW5, ODAD1, DEPDC7, MICALL2, SLC43A1, SLC6A20, RASA1, SLC45A4, NTAQ1, CGREF1, MICB, LSM11, PJA1, C2orf49, HRAS, KCNC3, MT2A, LRIT3, SHISA5, SLC25A40, H2AFX, PTPRA, RILPL2, ZC3CH13, ZHX2, CDLN4, ERH, GYPC, MTA2, NDUFV2, SDF4, and UBE2 W.

Embodiment 3 is the isolated set of probes of embodiment 1 or 2, wherein the panel of biomarkers comprises at least three biomarkers, at least four biomarkers, at least five biomarkers, at least six biomarkers, at least seven biomarkers, at least eight biomarkers, at least nine biomarkers, or ten biomarkers selected from a biomarker signature as follows:
a) DGKH, GADD45B, KLF7, LYST, NR6A1, PYCARD, ROBO1, SLC22A20P, SLC24A3, and SLC45A4;
b) DGKH, DIP2B, EMP1, GADD45B, MT2A, NOTCH2, NR6A1, RORA, SLC22A20P, and SL24A3;
c) GADD45B, LYST, NOXA1, NR6A1, PYCARD, SLC22A20P, SLC24A3, and NTAQ1;
d) DGKH, KLF7, LYST, NR6A1, ROBO1, SLC24A3, and SLC6A20;
e) DGKH, EMP1, GADD45B, LYST, SLC22A20P, SLC24A3, and SLC6A20;
f) CUEDC1, DGKH, EMP1, LYST, NOXA1, SLC22A20P, and SLC6A20;
g) DGKH, KLF7, LYST, NR6A1, PRICKLE1, ROBO1, and SLC24A3;
h) DCLK2, GADD45B, LYST, NR6A1, SLC22A20P, SLC24A3, and NTAQ1;
i) GADD45B, KLF7, LYST, NR6A1, ROBO1, SLC22A20P, and SLC6A20;
j) DCLK2, GADD45B, LYST, NR6A1, SLC22A20P, and NTAQ1;
k) C12orf65, GADD45B, LRBA, LYST, PEX1, PRKAB2, and TYW5;
l) LYST, PEX1, ODAD1, DEPDC7, MICALL2, SLC43A1, and SLC6A20;
m) C12orf65, GADD45B, LYST, PEX1, RASA1, SLC45A4, and NTAQ1;
n) TYW5, DEPDC7, SLC43A1, CGREF1, MICB, HRAS, and MT2A;
o) LRBA, LYST, PEX1, DEPDC7, SLC43A1, MICB, and C2orf49;
p) GADD45B, PEX1, DEPDC7, SLC43A1, LSM11, and PJA1;
q) DEPDC7, SLC43A1, MICB, C2orf49, HRAS, KCNC3, and MT2A;
r) LYST, DEPDC7, SLC43A1, SLC6A20, MICB, and HRAS;
S) PEX1, MICALL2, SLC43A1, RASA1, KCNC3, and LRIT3;
t) GADD45B, SLC43A1, SLC45A4, KCNC3, SHISA5, and SLC25A40;
u) GADD45B, H2AF, PTPRA, RILPL2, RORA, ZC3H13, and ZHX2; or
v) CLDN4, ERH, GADD45B, GYPC, H2AFX, MT2A, NDUFV2, SDF4, and UBE2 W.

Embodiment 4 is the isolated set of probes of any one of embodiments 1 to 3, wherein the probe is selected from the group consisting of an aptamer, an antibody, an affibody, a protein, an organic molecule, and a nucleic acid.

Embodiment 5 is a method for predicting disease prognosis and/or a treatment outcome for a subject diagnosed with cancer, the method comprising:
a) obtaining a sample from the subject;
b) contacting the sample with the isolated set of probes of any one of embodiments 1 to 4 to detect a panel of biomarkers in the sample; and
c) analyzing a pattern of the panel of biomarkers to determine a risk score for the subject.

Embodiment 6 is the method of embodiment 5, wherein the method further comprises:
d) classifying the subject as high risk or low risk based on the risk score.

Embodiment 7 is the method of embodiment 5 or 6, wherein the cancer is breast cancer.

Embodiment 8 is the method of embodiment 7, wherein the breast cancer is triple-negative breast cancer (TNBC).

Embodiment 9 is the method of embodiment 8, wherein the TNBC is early-stage TNBC.

Embodiment 10 is the method of embodiment 8 or 9, further comprising treating the TNBC in the subject based on the classification of the subject.

Embodiment 11 is the method of any one of embodiments 5 to 10, wherein the subject is high risk and the method further comprises an advanced, strengthened, or standard form of treatment for TNBC comprising surgery and/or administering chemotherapeutic agents, radiotherapy, immunotherapeutic agents, any novel therapeutics, or a combination of treatments.

Embodiment 11a is the method of any one of embodiments 5 to 10, wherein the subject is high risk and the method further comprises an advanced, strengthened, or standard form of treatment for TNBC comprising surgery and/or administering further chemotherapeutic agents, radiotherapy, immunotherapeutic agents, any novel therapeutics, or a combination of treatments as a neoadjuvant treatment, an adjuvant treatment, and/or a maintenance treatment.

Embodiment 12 is the method of embodiment 11 or 11a, wherein the immunotherapeutic agent is an immune checkpoint inhibitor.

Embodiment 13 is the method of embodiment 12, wherein the immune checkpoint inhibitor comprises pembrolizumab, atezolizumab, nivolumab, ipilimumab, durvalumab, and/or avelumab.

Embodiment 14 is the method of embodiment 11 or 11a, wherein the chemotherapeutic agents comprise capecitabine, doxorubicin, cyclophosphamide, docetaxel, olaparib, carboplatin, paclitaxel, epirubicin, methotrexate, and/or fluorouracil.

Embodiment 15 is the method of any one of embodiments 5 to 10, wherein the subject is low risk and the method further comprises administering the standard or attenuated treatment for TNBC comprising surgery only or surgery and/or administering chemotherapeutic agents, radiotherapy, immunotherapy, any novel treatment, or a combination of treatments.

Embodiment 15a is the method of any one of embodiments 5 to 10, wherein the subject is low risk and the method further comprises administering the standard or attenuated treatment for TNBC comprising surgery only or surgery and administering further chemotherapeutic agents, radiotherapy, immunotherapy, any novel treatment, or a combination of treatments as a neoadjuvant treatment, an adjuvant treatment, and/or a maintenance treatment.

Embodiment 16 is method of embodiment 15 or 15a, wherein the immunotherapeutic agent is an immune checkpoint inhibitor.

Embodiment 17 is the method of embodiment 16, wherein the immune checkpoint inhibitor comprises pembrolizumab, atezolizumab, nivolumab, ipilimumab, durvalumab, and/or avelumab.

Embodiment 18 is the method of embodiment 15 or 15a, wherein the chemotherapeutic agents comprise capecitabine, doxorubicin, cyclophosphamide, docetaxel, olaparib, carboplatin, paclitaxel, epirubicin, methotrexate, and/or fluorouracil.

Embodiment 19 is the method of any one of embodiments 5 to 18, wherein the sample is a tissue sample, a blood sample, or a urine sample.

Embodiment 20 is the method of embodiment 19, wherein the tissue sample is a fresh frozen tumor tissue sample or a fixed formalin paraffin embedded tumor tissue sample.

Embodiment 21 is a kit for predicting disease prognosis and/or a treatment outcome for a subject diagnosed with triple negative breast cancer (TNBC), the kit comprising:
a) an isolated set of probes capable of detecting a panel of biomarkers comprising at least two biomarkers selected from the group consisting of ankyrin repeat domain 36 (ANKRD36), ankyrin repeat domain 36B pseudogene 2 (ANKRD36BP2), B-box and SPRY domain-containing (BSPRY), chromosome 12 open reading frame 65 (C12orf65), chromosome 2 open reading frame 49 (C2orf49), chromosome 1 open reading frame 198 (C1orf198), coiled-coil domain containing 114 (CCDC114), claudin 4 (CLDN4), CUE domain containing 1 (CUEDC1), outer dynein arm docking complex subunit 1 (ODAD1), cell growth regulator with EF-hand domain 1 (CGREF1), DEP domain containing 7 (DEPDC7), doublecortin like kinase 2 (DCLK2), diacylglycerol kinase (DGKH), disco interacting protein 2 homolog B (DIP2B), disrupted-in-schizophrenia 1 (DISC1), epithelial membrane protein 1 (EMP1), ERH mRNA splicing and mitosis factor (ERH), growth arrest and DNA damage inducible beta (GADD45B), glutaminase (GLS), grainyhead like transcription factor 1 (GRHL1), glycophorin C (GYPC), H2A histone family member X (H2AFX), HRas proto-oncogene (HRAS), intracellular adhesion molecule 1 (ICAM1), interphotoreceptor matrix proteoglycan 2 (IMPG2), potassium voltage-gated channel subfamily C member 3 (KCNC3), kruppel like factor 6 (KLF6), kruppel like factor 7 (KLF7), keratin 17 (KRT17), LON peptidase N-terminal domain and RING finger protein 2 (LONRF2), LPS responsive beige-like anchor protein (LRBA), leucine rich repeat, Ig-like and transmembrane domains 3 (LRIT3), leucine rich repeat containing 37B (LRRC37B), LSM11, U7 small nuclear RNA associated (LSM11), lysosomal trafficking regulator (LYST), metastasis associated lung adenocarcinoma transcript 1 (MALAT1), minichromosome maintenance complex component 3 associated protein antisense RNA 1 (MCM3AP_AS1), MICAL like 2 (MICALL2), MHC class I polypeptide-related sequence B (MICB), metallothionein 2A (MT2A), myelin expression factor 2 (MYEF2), NEDD4 binding protein 3 (N4BP3), neuroblastoma breakpoint family member 20 (NBPF20), NADH: Ubiquinone Oxidoreductase Core Subunit V2 (NDUFV2), neurogenic locus notch homolog protein 2 (NOTCH2), NADPH oxidase activator 1 (NOXA1), natriuretic peptide receptor 3 (NPR3), nuclear receptor subfamily 6 group A member 1 (NR6A1), P21 (RAC1) activated kinase 3 (PAK3), pantothenate kinase 3 (PANK3), par-6 family cell polarity regulator beta (PARD6B), peroxisomal biogenesis factor 1 (PEX1), piggyBac transposable element derived 4 (PGBD4), praja ring finger ubiquitin ligase 1 (PJA1), pleckstrin homology and FYVE domain containing 1 (PLEKHF1), purine nucleoside phosphorylase (PNP), protein tyrosine phosphatase receptor type A (PTPRA), protein phosphatase, Mg2+/Mn2+ dependent 1K (PPM1K), prickle planar cell polarity protein 1 (PRICKLE1), protein kinase AMP-activated non-catalytic subunit beta 2 (PRKAB2), PYD and CARD domain containing (PYCARD), RAS p21 protein activator 1 (RASA1), RASD family member 2 (RASD2), RAS guanyl releasing protein 1 (RASGRP1), rhophilin RHO GTPase binding protein 2 (RHPN2), Rab interacting lysosomal protein like 2 (RILPL2), roundabout guidance receptor 1 (ROBO1), RAR related orphan receptor A (RORA), Stromal cell derived factor 4 (SDF4), SERTA domain containing 4 (SERTAD4), shisa family member 5 (SHISA5), signal induced proliferation associated 1 like 2 (SIPA1L2), solute carrier family 22 member 20 (SLC22A20P), solute carrier family 24 member 3 (SLC24A3), solute carrier family 2 member 12 (SLC2A12), solute carrier family 39 member 10 (SLC39A10), solute carrier family 25 member 40 (SLC25A40), solute carrier family 43 member 1 (SLC43A1), solute carrier family 45 member 4 (SLC45A4), solute carrier family 6 member 20 (SLC6A20), spectrin beta, erthrocytic (SPTB), stromal antigen 3-like 3 (STAG3L3), sushi domain containing 3 (SUSD3), TATA-Box binding protein associated factor 10 (TAF10), t-complex-associated testis expressed 3 (TCTE3), transmembrane channel like 7 (TMC7), transmembrane and coiled-coil domain family 2 (TMCC2), transcriptional repressor GATA binding 1 (TRPS1), tubulin tyrosine ligase like 4 (TTLL4), tRNA-yW synthesizing protein 5 (TYW5), ubiquitin conjugating enzyme E2 W (UBE2 W), WD repeat, sterile alpha motif and U-box domain containing 1 (WDSUB1), N-protein N-terminal glutamine amidohydrolase (WDYHV1), N-terminal glutamine amidase 1 (NTAQ1), zinc finger BED-type containing 6 (ZBED6), zinc finger and BTB domain containing 46 (ZBTB46), zinc finger CCCH-type containing 13 (ZC3H13), zinc fingers and homeoboxes 2 (ZHX2), zinc finger protein 217 (ZNF217), zinc finger protein 233 (ZNF233), zinc finger protein 248 (ZNF248), zinc finger protein 469 (ZNF469), and zinc finger protein 785 (ZNF785); and
b) instructions for use.

Embodiment 22 is the kit of embodiment 21, wherein the isolated set of probes capable of detecting a panel of biomarkers comprises at least three, at least four, at least five, at least six, at least seven, at least eight, at least nine, or ten biomarkers selected from a biomarker signature as follows:
a) DGKH, GADD45B, KLF7, LYST, NR6A1, PYCARD, ROBO1, SLC22A20P, SLC24A3, and SLC45A4;
b) DGKH, DIP2B, EMP1, GADD45B, MT2A, NOTCH2, NR6A1, RORA, SLC22A20P, and SL24A3;
c) GADD45B, LYST, NOXA1, NR6A1, PYCARD, SLC22A20P, SLC24A3, and NTAQ1;
d) DGKH, KLF7, LYST, NR6A1, ROBO1, SLC24A3, and SLC6A20;
e) DGKH, EMP1, GADD45B, LYST, SLC22A20P, SLC24A3, and SLC6A20;
f) CUEDC1, DGKH, EMP1, LYST, NOXA1, SLC22A20P, and SLC6A20;
g) DGKH, KLF7, LYST, NR6A1, PRICKLE1, ROBO1, and SLC24A3;
h) DCLK2, GADD45B, LYST, NR6A1, SLC22A20P, SLC24A3, and NTAQ1;
i) GADD45B, KLF7, LYST, NR6A1, ROBO1, SLC22A20P, and SLC6A20;
j) DCLK2, GADD45B, LYST, NR6A1, SLC22A20P, and NTAQ1;
k) C12orf65, GADD45B, LRBA, LYST, PEX1, PRKAB2, and TYW5;
l) LYST, PEX1, ODAD1, DEPDC7, MICALL2, SLC43A1, and SLC6A20;
m) C12orf65, GADD45B, LYST, PEX1, RASA1, SLC45A4, and NTAQ1;
n) TYW5, DEPDC7, SLC43A1, CGREF1, MICB, HRAS, and MT2A;
o) LRBA, LYST, PEX1, DEPDC7, SLC43A1, MICB, and C2orf49;
p) GADD45B, PEX1, DEPDC7, SLC43A1, LSM11, and PJA1;
q) DEPDC7, SLC43A1, MICB, C2orf49, HRAS, KCNC3, and MT2A;
r) LYST, DEPDC7, SLC43A1, SLC6A20, MICB, and HRAS;
s) PEX1, MICALL2, SLC43A1, RASA1, KCNC3, and LRIT3;
t) GADD45B, SLC43A1, SLC45A4, KCNC3, SHISA5, and SLC25A40;
u) GADD45B, H2AF, PTPRA, RILPL2, RORA, ZC3H13, and ZHX2; or
v) CLDN4, ERH, GADD45B, GYPC, H2AFX, MT2A, NDUFV2, SDF4, and UBE2 W.

Embodiment 23 is the kit of embodiment 21 or 22, wherein the isolated set of probes capable of detecting a panel of biomarkers comprises the biomarker signature of DGKH, GADD45B, KLF7, LYST, NR6A1, PYCARD, ROBO1, SLC22A20P, SLC24A3, and SLC45A4.

Embodiment 24 is the kit of embodiment 21 or 22, wherein the isolated set of probes capable of detecting a panel of biomarkers comprises the biomarker signature of C12orf65, GADD45B, LRBA, LYST, PEX1, PRKAB2, and TYW5.

Embodiment 25 is the kit of embodiment 21 or 22, wherein the isolated set of probes capable of detecting a panel of biomarkers comprises the biomarker signature of GADD45B, H2AF, PTPRA, RILPL2, RORA, ZC3H13, and ZHX2.

Embodiment 26 is the kit of embodiment 21 or 22, wherein the isolated set of probes capable of detecting a panel of biomarkers comprises the biomarker signature of CLDN4, ERH, GADD45B, GYPC, H2AFX, MT2A, NDUFV2, SDF4, and UBE2 W.

Embodiment 27 is the kit of any one of embodiments 21 to 26, wherein the probe is selected from the group consisting of an aptamer, an antibody, an affibody, a peptide, a protein, an organic molecule, and a nucleic acid.

### EXAMPLES

### Materials and Methods

The study included 184 patients with early-stage triple negative breast cancer (TNBC); 76 patients in the training cohort were from the National Cancer Center Korea (NCC), and 108 patients in the validation cohort were from Samsung Medical Center (SMC). All patients were eligible if they were ≥18-years-old with early-stage TNBCs (stage I-III), for whom a histological biopsy could be safely obtained and standard systemic chemotherapy and loco-regional treatment including surgery and radiation were applied. Tumor samples were identified as TNBCs according to the American Society of Clinical Oncology/College of American Pathologists (ASCO/CAP) guidelines for the assessment of ER, PR, and HER2 (12, 13). The training cohort consisted of 15 patients who received neoadjuvant chemotherapy and 61 patients who underwent primary surgery followed by adjuvant chemotherapy for early stage TNBC between March 2002 and August 2018. The validation cohort included 73 patients who had received neoadjuvant chemotherapy and 35 patients who received adjuvant chemotherapy after surgery between July 2011 and November 2017. In the validation cohort, 42 specimens of the neoadjuvant chemotherapy group were biopsy tissue before neoadjuvant chemotherapy, while the other specimens were surgical tissues. All specimens were fresh-frozen. All patients provided written informed consent, and the study protocol was approved by the institutional review boards of the National Cancer Center Korea and Samsung Medical Center (NCC IRB #2012-08-065, SMC IRB #2014-11-015).

### Complete Clinical Information and Outcome

Clinical data, including the date of diagnosis, clinical and surgical stages, response to neoadjuvant chemotherapy, recurrence, and survival, were collected from medical records. Invasive disease-free survival (IDFS) was defined as the time from diagnosis of primary breast cancer to invasive breast cancer recurrence or death from any cause.

RNA Extraction and cDNA Library Preparation

Total RNA was extracted according to the manufacturer's protocol using TRIzol reagent (Invitrogen, Thermo Fisher Scientific, CA, USA) and AllPrep DNA/RNA Mini kit (Qiagen, Hilden, Germany). DNA contamination was eliminated using DNase. RNA quality control was evaluated by RNA integrity number (RIN) using an Agilent 2100 Bioanalyzer (Agilent Technologies, Waldbronn, Germany), and RNA with a RIN exceeding 8 passed quality control. The cDNA library was prepared according to the manufacturer's protocol (TruSeq Stranded mRNA Sample Preparation Guide, Part #15031047 Rev. E) using a TruSeq Stranded mRNA LT Sample Prep Kit (Illumina, CA, USA).

### RNA Sequencing and Quality Control

For the training cohort, paired-end sequencing was conducted using the prepared cDNA library for RNA sequencing using an Illumina HiSeq 4000 sequencer (Illumina, CA, USA). In RNA sequencing quality control, artifacts including adaptor sequences, contaminant DNA, and PCR duplicates were eliminated to reduce the bias of sequencing data. After quality control of sequencing data, aligned reads were generated by mapping sequencing data on the reference genome using the HISAT2 program (GitHub, http://daehwankimlab.github.io/hisat2/). With generated aligned reads, transcript assembly was conducted using StringTie (https://ccb.jhu.edu/software/stringtie/). Based on the transcript quantification of each sample, expression levels were normalized to transcript length and depth of coverage. Through normalization, expression profiles were extracted as fragments per kilobase of transcript per million mapped reads (FPKM).

For the validation cohort, sequencing libraries were prepared using fresh frozen tissues with TruSeq RNA Sample Prep Kit v2 (Illumina Inc.) following the manufacturer's protocols. Sequencing of the RNA libraries was performed on a HiSeq 2500 sequencing platform (Illumina Inc.). After trimming poor-quality bases from the FASTQ files, the reads were aligned to the human reference genome (hg19) using STAR v.2.5 and estimated gene expression in terms of fragments per kilobase of exon per million (FPKM) using RSEM v.1.3. The quality control of sequencing results was assessed using RNA-SeQC (v1.1.8).

For comparison between tumors and non-tumors, non-tumor data were collected from Gene Expression Omnibus (GEO, ncbi.nlm.nih.gov/geo). GSE58135 (GEO accession id) was selected as the non-tumor group in GEO, and it had non-tumor RNA sequencing data of 21 patients with TNBC. In non-tumor data, data with a failed status or with values under 1.0×10⁻⁶ were excepted.

### Combination Gene Analysis

By matching the genes of tumors and non-tumors, 10,856 genes were found in both groups. With 10,856 genes, the differentially expressed genes (DEGs) were analyzed. DEGs were screened to meet one of the following conditions: 1) statistically significant difference comparing tumors with non-tumors and 2) statistically significant difference comparing the patients who exhibited recurrence/metastasis after surgical resection with the patients without recurrence/metastasis. The previously screened DEGs were further shortlisted using Cox regression analysis for recurrence/metastasis. Before combining the DEGs, the Cox regression coefficient of each gene was identified and weighted gene expression with the corresponding coefficient value. The gene signature was calculated using equation (14):

$$\sum ( correlation regression coefficient \times gene expression value )$$

The number of shortlisted DEGs analyzed in combination and the total number of gene combinations is given by equation (14):

$${\sum }_{k = 1}^{n} \frac{n !}{K ! ( n - k ) !}$$

where, n is the total number of shortlisted DEGs, and k is the number of genes included in the combinations.

### Pre-Validation of the Candidate Gene Signatures by Cross Validation of Machine Learning

Candidate gene signatures (achieving p-value<0.05, area under the curve (AUC)>0.90, sensitivity >90%, and specificity >90%) were ranked by k-fold cross validation to identify the optimal gene combination. The patients were randomly separated by 2 folds (training set and test set) 300 times (14).

### Signal Transduction Pathway Analysis Based on Meta-Analysis

Signal transduction analysis was performed using the CBS Probe PINGS™ (Reg. No. 2008-01-129-000568; CbsBioscience, Daejeon, Korea), which consists of five modules (PPI module, Path-Finder module, Path-Linker module, Path-maker module, and Path-Lister module) (14). For gene signature validation, signal transduction was analyzed for pathways related to each patient's DEGs compared recurrence/metastasis and non-recurrence/non-metastasis and for pathways related to gene signature. The genes were mapped to the signal transduction pathways obtained from the Kyoto Encyclopedia of Genes and Genomes (KEGG) database. The top 10 signal transduction pathways were selected for each patient's DEGs and for gene signature according to the weight of the number of interactions and interacting genes. Ten pathways related to each patient's DEGs and gene signature-related pathways were compared. For each signal transduction pathway selected, in signal transduction pathway analysis, the gene interaction frequency ratio was computed, which is a score of interacting genes with signature genes in gene signature validation. By applying 100% gene interaction frequency to the highest probability of gene interaction within each signal transduction pathway, the top 10 high interaction frequency genes were selected. In addition, 10 high interaction frequency genes related to each patient's DEGs and gene signatures related to high interaction frequency genes were compared.

### Molecular Subtype Classification Using PAM 50 Analysis

PAM 50 call analysis was performed using R v.3.4.3 (R Development Core Team, r-project.org) using the published R script (15, 16). The median of the FPKM data and the centroid data for PAM50 were set to the library. By inputting patients' RNA sequencing data, intrinsic subtypes of patients and risk of recurrence (ROR) score were analyzed by R using the library settings described above. Using KM analysis, the prognostic power of the PAM 50 call was analyzed using ROR-S.

### T-Cell Receptor Diversity Analysis

T cell receptor (TCR) profiles were obtained using MiXCR 2.1.3 (GitHub, github.com/milaboratory/mixcr) using RNA sequencing data (17, 18). RNA sequencing data were aligned to all the IG/TCR loci. After two rounds of contig assembly, the V/J junctions of TCRs were extended. The assembled clonotypes were exported. TCR diversity was analyzed with the T cell receptor beta locus (TCRB) using the Shannon index. The Shannon index is given by the following equation:

$$- {\sum }_{i = 1}^{s} \frac{n i}{N} ln \frac{n i}{N}$$

where, s is the number of different clonotypes, ni is the clonal size of the ith clonotype, and N is the total number of TCRB sequences analyzed. Using KM analysis, the prognostic power of TCRB diversity was analyzed.

### Statistical Analysis

Clinicopathological variables between the training and validation cohorts were evaluated using chi-square tests or Fisher's exact tests. Gene expression data were tested for normality using the Shapiro-Wilk test. As the data did not meet normality assumptions, significant differences between the responders and non-responders were evaluated using the Wilcoxon test. Receiver operating characteristic (ROC) curve analysis was used to determine the accuracy of threshold values for classifying recurrence/metastasis and non-recurrence/non-metastasis using gene signatures. Kaplan-Meier survival (KM) curves were calculated using death and invasive disease as endpoints in invasive disease-free survival (iDFS). The difference in KM curves was examined using the log-rank test, and the difference in hazard ratio was examined using Cox regression analysis. Candidate gene signatures were analyzed using Cox regression to understand the relationships between the recurrence/metastasis, classification, and clinicopathological variables. Significance was set at p<0.05. All statistical analyses were performed using R v.3.4.3 software (R Development Core Team, r-project.org/).

### Example 1: Results for Analysis 1

### Clinical Characteristics of Patients

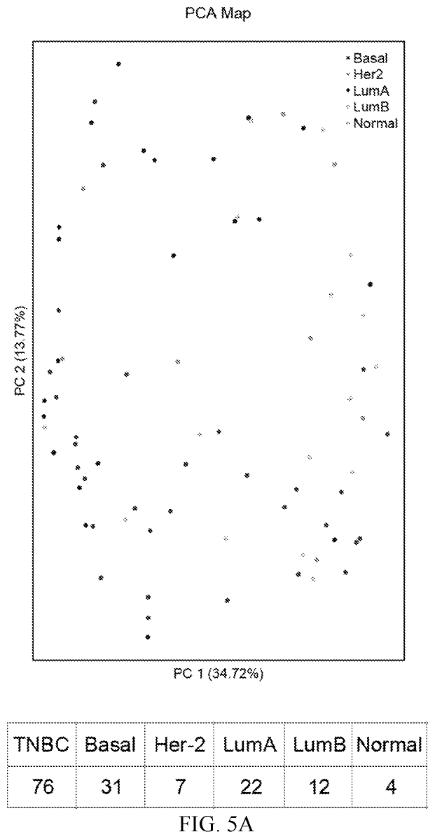

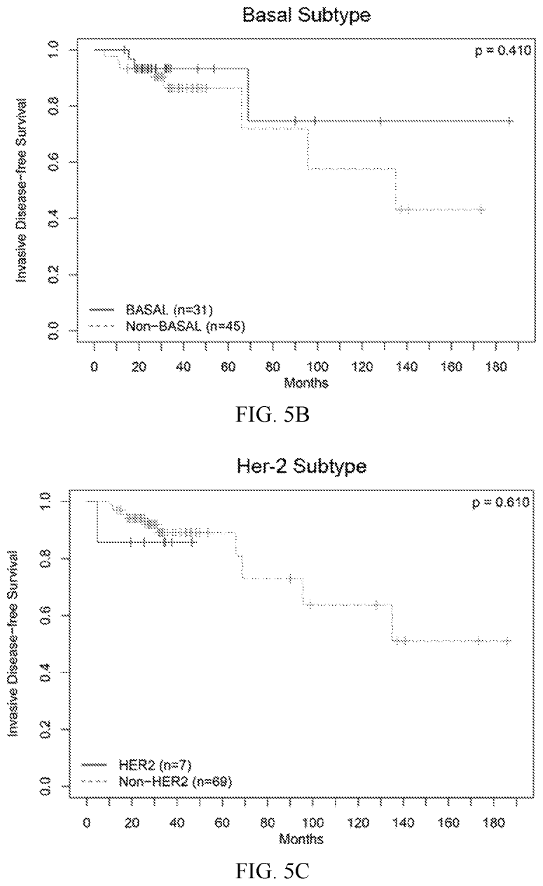

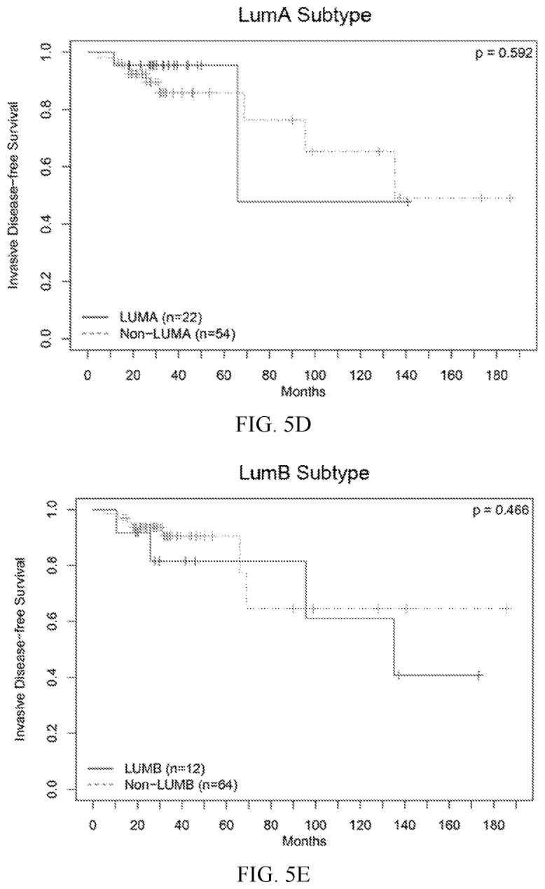

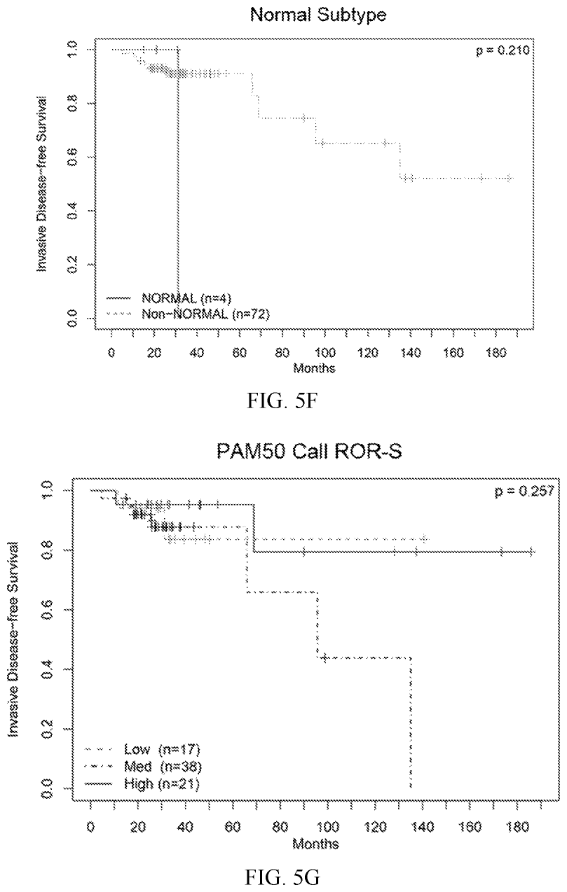

Of the 184 patients, 76 were in the training cohort and 108 in the validation cohort. The clinical characteristics of the patients in the training and validation cohorts are summarized in Table 1. Overall, the patients in the very young age group were not significantly different between the cohorts. Patients in the training cohort were more likely to have earlier-stage diseases than the validation cohort; however, the distribution of stage among the patients who underwent primary surgery or who had residual tumors after neoadjuvant chemotherapy was not different between the two groups. For adjuvant chemotherapy, the TAC (taxotere, adriamycin, and cyclophosphamide) regimen was used more in the validation cohort, probably because there were more advanced stage patients. A schematic representation of the patients and samples is shown in FIGS. 5A-5G.

TABLE 1
Pathological Baselines of the training cohort and validation cohort.

| | Training cohort (%) | | | Validation cohort (%) | | | Training |
|---|---|---|---|---|---|---|---|
| | | Primary | Residual | | Primary | Residual | vs |
| Pathologic | Total | tumors | tumors | Total | tumors | tumors | validation |
| parameters | (n = 76) | (n = 61) | (n = 15) | (n = 108) | (n = 77) | (n = 31) | p-value* |
| Age (range) | | | | | | | 0.1465 |
| 35 years | 17 (22.4) | 10 (16.4) | 7 (46.7) | 36 (33.3) | 24 (31.2) | 12 (38.7) | |
| ≥35 years | 59 (77.6) | 51 (83.6) | 8 (53.3) | 72 (66.7) | 53 (68.8) | 19 (61.3) | |
| TNM stage | | | | | | | 3.48 × 10⁻⁵ |
| I | 29 (38.2) | 29 (47.5) | — | 18 (16.7) | 18 (23.4) | | |
| II | 42 (55.3) | 32 (52.5) | 10 (66.7) | 57 (52.8) | 44 (57.1) | 13 (41.9) | |
| III | 5 (6.6) | — | 5 (33.3) | 33 (30.6) | 15 (19.5) | 18 (58.1) | |
| Systemic | | | | | | | — |
| chemotherapy | | | | | | | |
| Neo-adjuvant | 15 (19.7) | — | 15 (100.0) | 73 (67.6) | 42 (54.5) | 31 (100.0) | |
| AC | 2 (13.3) | — | 2 (13.3) | 2 (2.7) | — | 2 (6.5) | |
| AC-D | 6 (40.0) | — | 6 (40.0) | 64 (87.7) | 35 (83.3) | 29 (93.5) | |
| AC-wP | — | — | — | 7 (9.6) | 7 (16.7) | — | |
| AC-PC | 4 (26.7) | — | 4 (26.7) | — | — | — | |
| PCarbo | 2 (13.3) | — | 2 (13.3) | — | — | — | |
| DA | 1 (6.7) | — | 1 (6.7) | — | — | — | |
| Adjuvant | 61 (80.3) | 61 (100.0) | — | 35 (32.4) | 35 (45.5) | — | |
| AC | 6 (9.8) | 6 (9.8) | — | 2 (5.7) | 2 (5.7) | — | |
| TC | 14 (23.0) | 14 (23.0) | — | 1 (2.9) | 1 (2.9) | — | |
| FAC | 14 (23.0) | 14 (23.0) | — | 3 (8.6) | 3 (8.6) | — | |
| AC-D | 7 (11.5) | 7 (11.5) | — | 5 (14.3) | 5 (14.3) | — | |
| AC-wP | 6 (9.8) | 6 (9.8) | — | 4 (11.4) | 4 (11.4) | — | |
| AC-PC | 3 (4.9) | 3 (4.9) | — | — | — | — | |
| TAC | 11 (18.0) | 11 (18.0) | — | 20 (57.1) | 20 (57.1) | — | |
| Event | | | | | | | 0.4057 |
| Presence | 11 (14.5) | 7 (11.5) | 4 (26.7) | 22 (20.4) | 8 (10.4) | 14 (45.2) | |
| Absence | 65 (85.5) | 54 (88.5) | 11 (73.3) | 86 (79.6) | 69 (89.6) | 17 (54.8) | |
| Specimen | | | | | | | |
| Core Biopsy | — | — | — | 42 (38.9) | 42 (54.5) | — | |
| Surgery | 76 (100.0) | 61 (100.0) | 15 (100.0) | 66 (61.1) | 35 (45.5) | 31 (100.0) | |
| Follow-up period, | | | | | | | |
| months | | | | | | | |
| Median | 29.5 | 28.5 | 35.6 | 45.6 | 47.4 | 40.1 | |
| Range | 4.6-185.9 | 11.5-185.9 | 4.6-128.1 | 6.6-74.5 | 12.9-74.5 | 6.6-73.6 | |

TNM, tumor-node-metastasis (AJCC stage); pCR, pathologic complete response; Event, Recurrence or Metastasis; AC, Adriamycin, cyclophosphamide; AC-D, AC followed by docetaxel; TC, Taxotere and cyclophosphamide; FAC, 5-FU, Adriamycin, cyclophosphamide; AC-wP, AC followed by weekly paclitaxel; AC-PC, AC followed by paclitaxel and carboplatin; PCarbo, paclitaxel and carboplatin; DA, docetaxel and adriamycin; TAC, taxotere, and AC.
*p-values were calculated using Fisher's exact test.

### DEG Analyses of Tumor Versus Non-Tumor, and Recurrence and Metastasis Versus Non-Recurrence and Non-Metastasis

In the DEG analysis of tumor versus non-tumor, 9,741 genes were significantly differentially expressed by more than 1.5-fold changes in 10,856 genes. DEG analysis of primary tumors between relapsed and non-relapsed patients revealed 141 out of 10,856 genes showing significant differences in expression by 1.5-fold. Subsequently, out of 10,856 genes, 587 genes were statistically significant in the single Cox analysis. In the DEG analysis in three ways, 70 DEGs overlapped (Table 2).

TABLE 2
DEGS correlated in prognostic of TNBC

| | | | | Event vs | Event vs | |
|---|---|---|---|---|---|---|
| | | NT vs T | NT vs T | non-event | non-event | Cox |
| No | Gene | p-value* | Fold Change | p-value* | Fold Change | p-value |
| 1 | ANKRD36 | 1.74E−03 | 1.84 | 1.55E−02 | 2.02 | 3.91E−04 |
| 2 | ANKRD36BP2 | 6.21E−11 | 18.48 | 7.87E−03 | 2.34 | 3.28E−04 |
| 3 | BSPRY | 1.40E−11 | 17.68 | 2.39E−02 | 2.20 | 2.63E−03 |
| 4 | C12orf65 | 3.02E−12 | 4.32 | 6.49E−04 | −1.59 | 1.60E−02 |
| 5 | C2orf49 | 2.84E−12 | 10.90 | 2.68E−02 | 1.53 | 6.54E−03 |
| 6 | ODAD1 | 8.71E−03 | 1.91 | 1.26E−02 | 2.19 | 2.20E−04 |
| 7 | CGREF1 | 4.60E−10 | 7.65 | 1.37E−02 | 1.53 | 3.02E−02 |
| 8 | DEPDC7 | 2.84E−12 | 65.81 | 8.97E−03 | −1.84 | 2.71E−02 |
| 9 | DGKH | 1.32E−10 | 8.43 | 6.31E−03 | 1.73 | 4.01E−03 |
| 10 | DIP2B | 2.84E−12 | 6.36 | 1.36E−03 | 1.84 | 3.30E−03 |
| 11 | EMP1 | 6.35E−08 | 3.00 | 2.68E−02 | 1.80 | 1.54E−03 |
| 12 | GADD45B | 4.63E−11 | 6.12 | 1.97E−02 | 1.68 | 9.25E−03 |
| 13 | GLS | 2.84E−12 | 17.00 | 4.38E−03 | 1.63 | 1.76E−02 |
| 14 | GRHL1 | 1.04E−11 | 14.72 | 1.11E−02 | 1.56 | 2.17E−02 |
| 15 | HRAS | 9.15E−04 | 1.65 | 3.11E−02 | −1.59 | 3.83E−02 |
| 16 | ICAM1 | 5.28E−12 | 11.74 | 2.58E−02 | −1.99 | 3.68E−02 |
| 17 | IMPG2 | 4.35E−10 | 1.89 | 3.48E−02 | 1.52 | 5.16E−03 |
| 18 | KCNC3 | 3.46E−09 | 7.41 | 2.48E−02 | 1.76 | 3.41E−02 |
| 19 | KRT17 | 4.35E−02 | 3.25 | 1.37E−02 | −4.71 | 4.38E−02 |
| 20 | LONRF2 | 7.12E−05 | 6.75 | 2.21E−02 | 3.48 | 3.50E−04 |
| 21 | LRBA | 6.43E−10 | −3.32 | 3.81E−03 | 1.58 | 3.17E−02 |
| 22 | LRIT3 | 6.39E−11 | 11.19 | 2.89E−02 | 1.84 | 7.96E−04 |
| 23 | LRRC37B | 2.84E−12 | 7.54 | 4.31E−02 | 1.50 | 6.95E−04 |
| 24 | LSM11 | 2.84E−12 | 13.74 | 1.16E−02 | 1.50 | 3.02E−04 |
| 25 | LYST | 2.48E−10 | 3.41 | 6.31E−03 | 2.01 | 8.50E−05 |
| 26 | MALAT1 | 3.48E−10 | −3.29 | 3.87E−02 | 1.93 | 1.45E−03 |
| 27 | MCM3AP.AS1 | 6.58E−11 | 6.76 | 1.61E−02 | 1.71 | 1.45E−04 |
| 28 | MICALL2 | 2.47E−05 | 2.18 | 4.47E−02 | −1.63 | 2.74E−02 |
| 29 | MICB | 3.24E−11 | 9.68 | 4.79E−02 | −1.66 | 2.24E−02 |
| 30 | MT2A | 3.22E−12 | 30.27 | 4.81E−03 | 1.88 | 5.55E−04 |
| 31 | MYEF2 | 5.62E−12 | 19.55 | 4.96E−02 | 1.53 | 4.83E−02 |
| 32 | N4BP3 | 1.40E−11 | 6.20 | 1.31E−02 | 1.61 | 2.47E−02 |
| 33 | NOTCH2 | 9.75E−12 | 4.35 | 4.63E−02 | 2.00 | 2.41E−04 |
| 34 | PANK3 | 5.28E−12 | 8.33 | 1.43E−02 | 1.55 | 2.79E−02 |
| 35 | PEX1 | 2.84E−12 | 9.00 | 5.03E−03 | 1.82 | 2.75E−02 |
| 36 | PGBD4 | 2.84E−12 | 14.09 | 1.55E−02 | 1.78 | 4.51E−05 |
| 37 | PJA1 | 2.26E−09 | 6.07 | 4.02E−02 | −1.74 | 3.13E−02 |
| 38 | PLEKHF1 | 1.55E−09 | 4.48 | 9.37E−03 | −1.87 | 3.26E−02 |
| 39 | PNP | 3.02E−12 | 10.76 | 3.60E−02 | 1.59 | 2.93E−03 |
| 40 | PPM1K | 1.48E−10 | 3.10 | 7.54E−03 | 1.57 | 1.26E−03 |
| 41 | PRKAB2 | 1.04E−11 | 15.21 | 1.49E−02 | 1.52 | 9.50E−03 |
| 42 | RASA1 | 3.42E−12 | 4.80 | 2.48E−02 | 1.99 | 4.07E−04 |
| 43 | RASD2 | 4.39E−12 | 24.12 | 4.79E−02 | 2.26 | 7.87E−03 |
| 44 | RASGRP1 | 2.84E−12 | 43.46 | 1.31E−02 | 2.47 | 5.95E−05 |
| 45 | RHPN2 | 2.27E−11 | 13.39 | 1.58E−03 | 2.02 | 2.99E−02 |
| 46 | SERTAD4 | 6.58E−11 | 14.86 | 2.89E−02 | 1.94 | 1.15E−02 |
| 47 | SHISA5 | 5.87E−09 | 3.97 | 1.75E−02 | −1.63 | 1.25E−02 |
| 48 | SIPA1L2 | 8.12E−12 | 6.00 | 4.79E−02 | 1.60 | 1.13E−02 |
| 49 | SLC22A20P | 1.83E−07 | 4.94 | 1.21E−02 | 2.11 | 3.62E−03 |
| 50 | SLC25A40 | 2.84E−12 | 12.05 | 4.79E−02 | 1.56 | 5.77E−03 |
| 51 | SLC43A1 | 1.37E−03 | 1.67 | 1.16E−03 | −1.90 | 2.49E−02 |
| 52 | SLC45A4 | 3.06E−11 | 4.30 | 2.86E−03 | 1.92 | 6.55E−05 |
| 53 | SLC6A20 | 8.93E−03 | 2.63 | 2.30E−02 | 2.64 | 7.34E−03 |
| 54 | SPTB | 5.49E−04 | −1.50 | 4.63E−02 | 1.81 | 1.86E−02 |
| 55 | STAG3L3 | 3.64E−12 | 6.44 | 1.66E−03 | 1.72 | 1.26E−04 |
| 56 | SUSD3 | 9.98E−07 | 2.90 | 1.21E−02 | −1.76 | 4.83E−02 |
| 57 | TAF10 | 3.64E−12 | 4.42 | 5.03E−03 | −1.51 | 3.27E−02 |
| 58 | TMC7 | 4.12E−12 | 8.63 | 4.96E−02 | 1.76 | 2.99E−02 |
| 59 | TMCC2 | 4.11E−11 | 9.92 | 6.60E−03 | 2.02 | 2.91E−04 |
| 60 | TTLL4 | 7.40E−11 | 5.22 | 2.89E−02 | 1.56 | 1.14E−02 |
| 61 | TYW5 | 2.84E−12 | 11.58 | 2.03E−03 | 2.92 | 5.73E−03 |
| 62 | WDSUB1 | 2.84E−12 | 20.06 | 4.18E−03 | 1.54 | 7.29E−03 |
| 63 | NTAQ1 | 2.84E−12 | 15.31 | 1.61E−02 | 1.56 | 2.67E−02 |
| 64 | ZBED6 | 4.12E−12 | 11.78 | 2.58E−02 | 1.57 | 2.27E−02 |
| 65 | ZBTB46 | 3.63E−05 | 1.95 | 4.96E−02 | 1.94 | 4.22E−05 |
| 66 | ZNF217 | 2.84E−12 | 12.32 | 2.24E−03 | 1.60 | 6.61E−04 |
| 67 | ZNF233 | 8.10E−08 | 3.74 | 3.48E−02 | 1.50 | 2.98E−02 |
| 68 | ZNF248 | 8.31E−11 | 4.87 | 5.27E−03 | 1.58 | 3.09E−04 |
| 69 | ZNF469 | 2.56E−11 | 11.30 | 4.96E−02 | 2.62 | 9.26E−05 |
| 70 | ZNF785 | 1.57E−10 | 2.17 | 4.42E−04 | 1.56 | 4.91E−06 |

### Candidate Gene Signatures by Gene Combination and Selected Gene Signature by Cross Validation

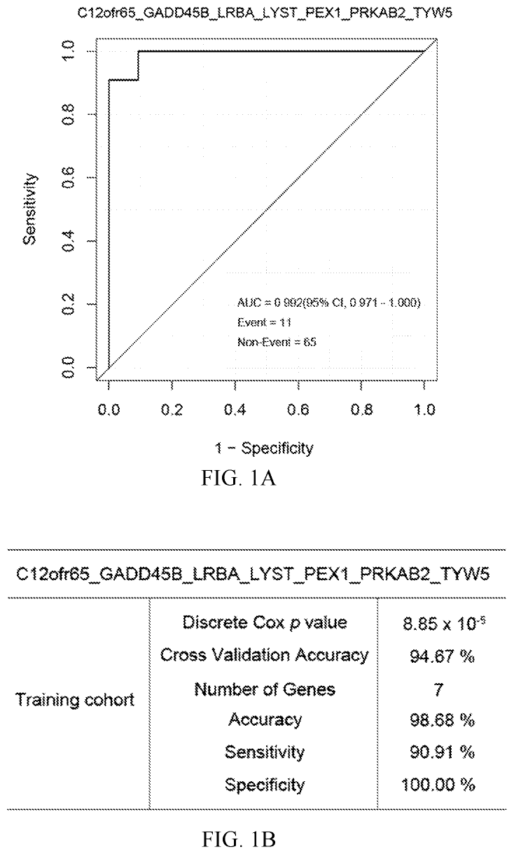

The top 10 candidate gene signatures were ranked with the AUC. Ten candidates had equal values that sensitivity of 90.91, specificity of 100.00, and accuracy of 98.68 respectively; however, the AUC of the candidates was different. The prognostic gene signature was selected using 2-fold cross-validation accuracy. The selected gene signature was C12orf65; GADD45B; LRBA; LYST; PEX1; PRKAB2; TYW5, showing 94.67% cross-validation accuracy, and it was statistically significant in the discrete Cox analysis. The risk score was calculated with a cut-off value of 4.043659 as follows: (−0.334912×C12orf65)+(0.018572×GADD45B)+(0.124030×LRBA)+(0.257051×LYST)+(0.046903×PEX1)+(0.220736×PRKAB2)+(0.083961×TYW5) (FIGS. 1A-1B, Table 3).

TABLE 3
Gene signature candidates as prognostic biomarker of TNBC

| | | No. of | Cross | | | | | Cont. |
|---|---|---|---|---|---|---|---|---|
| | | Comb. | Valid. | | | | | Cox |
| Rank | Gene Signature | genes | Acc. | AUC | Sens. | Spec. | Acc. | p-value |
| 1 | C12orf65; GADD45B; LRBA; LYST; | 7 | 94.67 | 0.992 | 90.91 | 100 | 98.68 | 7.68E−07 |
| | PEX1; PRKAB2; TYW5 | | | | | | | |
| 2 | ODAD1; DEPDC7; LYST; MICALL2; | 7 | 92.67 | 0.992 | 90.91 | 100 | 98.68 | 3.35E−07 |
| | PEX1; SLC43A1; SLC6A20 | | | | | | | |
| 3 | C12orf65; GADD45B; LYST; PEX1; | 7 | 92.67 | 0.992 | 90.91 | 100 | 98.68 | 3.57E−07 |
| | RASA1; SLC45A4; NTAQ1 | | | | | | | |
| 4 | CGREF1; DEPDC7; HRAS; MICB; | 7 | 90.33 | 0.992 | 90.91 | 100 | 98.68 | 4.90E−07 |
| | MT2A; SLC43A1; TYW5 | | | | | | | |
| 5 | C2orf49; DEPDC7; LRBA; LYST; | 7 | 88.33 | 0.992 | 90.91 | 100 | 98.68 | 3.80E−07 |
| | MICB; PEX1; SLC43A1 | | | | | | | |
| 6 | DEPDC7; GADD45B; LSM11; PEX1; | 6 | 86.33 | 0.992 | 90.91 | 100 | 98.68 | 6.98E−07 |
| | PJA1; SLC43A1 | | | | | | | |
| 7 | C2orf49; DEPDC7; HRAS; KCNC3; | 7 | 85.67 | 0.992 | 90.91 | 100 | 98.68 | 5.62E−07 |
| | MICB; MT2A; SLC43A1 | | | | | | | |
| 8 | DEPDC7; HRAS; LYST; MICB; | 6 | 94.33 | 0.990 | 90.91 | 100 | 98.68 | 1.63E−06 |
| | SLC43A1; SLC6A20 | | | | | | | |
| 9 | KCNC3; LRIT3; MICALL2; PEX1; | 6 | 91.33 | 0.990 | 90.91 | 100 | 98.68 | 1.06E−06 |
| | RASA1; SLC43A1 | | | | | | | |
| 10 | GADD45B; KCNC3; SHISA5; | 6 | 81.33 | 0.990 | 90.91 | 100 | 98.68 | 1.82E−06 |
| | SLC25A40; SLC43A1; SLC45A4 | | | | | | | |

AUC, Area under the curve; C12orf65, chromosome 12 open reading frame 65; GADD45B, Growth arrest and DNA damage inducible beta; LRBA, LPS responsive beige-like anchor protein; LYST, lysosomal trafficking regulator; PEX1, peroxisomal biogenesis factor 1; PRKAB2, protein kinase AMP-activated non-catalytic subunit beta 2; TYW5, tRNA-yW synthesizing protein 5; ODAD1; outer dynein arm docking complex subunit 1, DEPDC7, DEP domain containing 7; MICALL2; MICAL like 2, SLC43A1; solute carrier family 43 member 1, SLC6A20; solute carrier family 6 member 20, RASA1; RAS p21 protein activator 1, SLC45A4; solute carrier family 45 member 4, NTAQ1; N-terminal glutamine amidase 1, CGREF1; cell growth regulator with EF-hand domain 1, HRAS; HRas proto-oncogene, GTPase, MICB; MHC class I polypeptide-related sequence B, MT2A; metallothionein 2A, C2orf49; chromosome 2 open reading frame 49, LSM11; LSM11, U7 small nuclear RNA associated, PJA1; praja ring finger ubiquitin ligase 1, KCNC3; potassium voltage gated channel subfamily C member 3, LRIT3; leucine rich repeat, Ig-like and transmembrane domains 3, SHISA5; shisa family member 5, SLC25A40; solute carrier family 25 member 40

### Prognostic Significance of Gene Signature in the Training Cohort

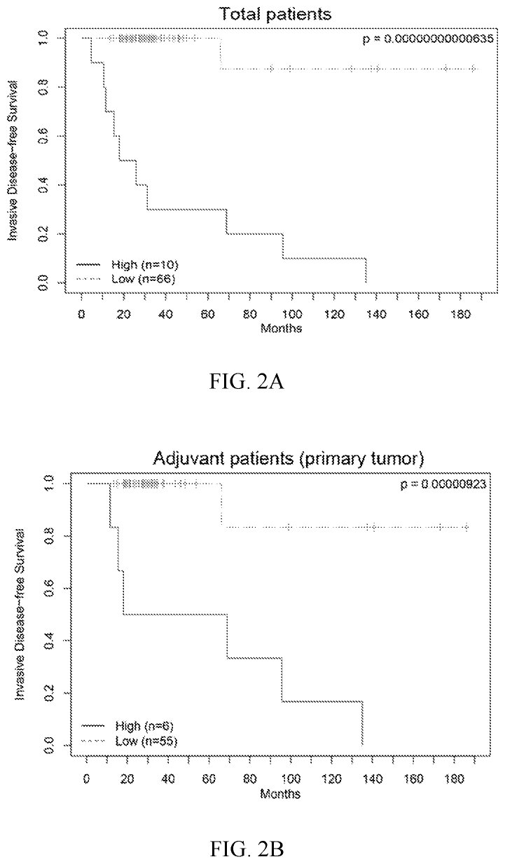

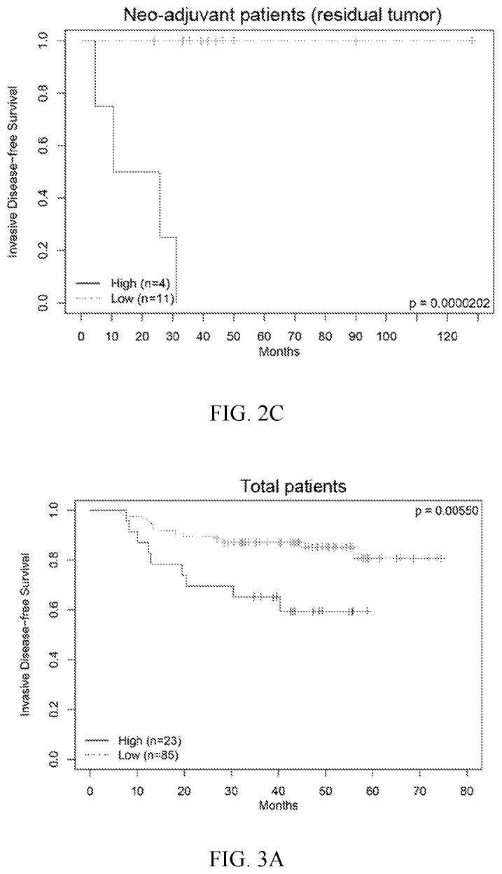

During the median follow-up of 29.5 months (range: 4.6-185.9), patients with tumors with high-risk gene signatures (n=10) showed significantly shorter invasive disease-free survival (iDFS) (median, 95% confidence interval [CI]; 21.9, 11.5—not reached) than those with low-risk signatures (n=66, median not reached, p=6.35×10⁻¹²) in the overall population (FIG. 2A). Further analysis in the separate group of patients who underwent primary surgery and in the patients with residual tumors after neoadjuvant chemotherapy showed similar results. Among patients who received primary surgery and patients with residual tumors after neoadjuvant chemotherapy, the median iDFS in the high-risk group was 43.4 months (95% CI: 15.5—not reached; p=0.00000923) and 18.2 months (95% CI: 4.6—not reached, p=0.000020), respectively, and the median iDFS in the low-risk group did not reach the median (FIG. 2B, C).

### Prognostic Significance of Gene Signatures in the Validation Cohort

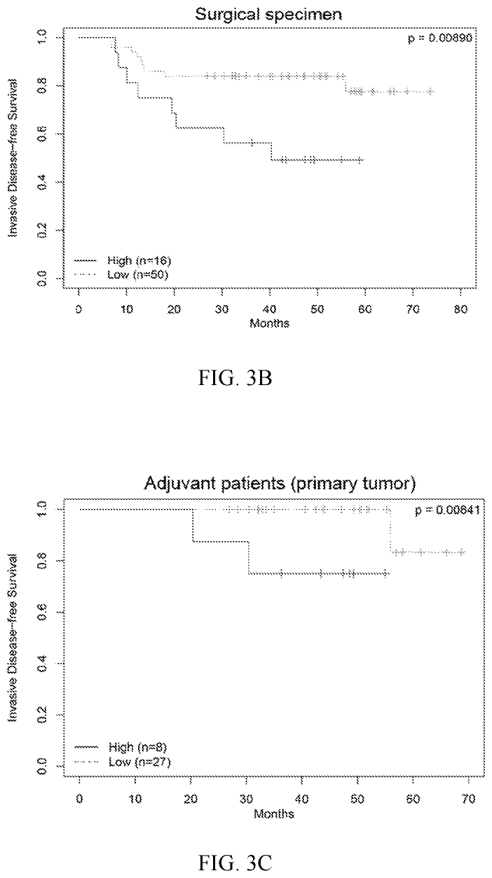

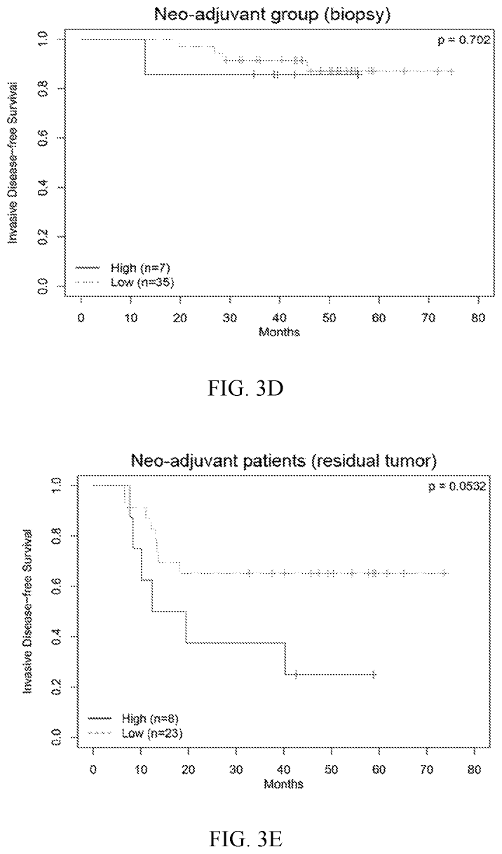

Median follow up time for validation cohort was 45.6 months (range; 6.6-74.5). In the overall validation cohort, although the median iDFS of the patients with high-risk genetic signatures was not reached, there was a significantly higher risk for recurrence or metastasis than patients with low-risk gene signatures (median iDFS not reached and p=0.0055 in log-rank test). When the patients were sub-divided according to the treatment sequence, the prognostic significance of the gene signatures in the surgical tissues from the patients who underwent primary surgery was consistent with the training cohort (p=0.0084); however, the median iDFS in the high-risk and low-risk groups was not yet reached. High-risk gene signatures were still valid in predicting the prognosis of patients with residual tumors after neoadjuvant chemotherapy; median iDFS in the high-risk group was 15.9 months (95% CI, 10.1—not reached), but it was not reached in the low-risk group (p=0.053). However, when the gene signatures were examined in the tissues obtained by core biopsy in the neoadjuvant chemotherapy group, the trend for prognostic significance in iDFS did not reach statistical significance (p=0.702). (FIGS. 3A-3E).

### Investigation of Other Potential Prognostic Factors

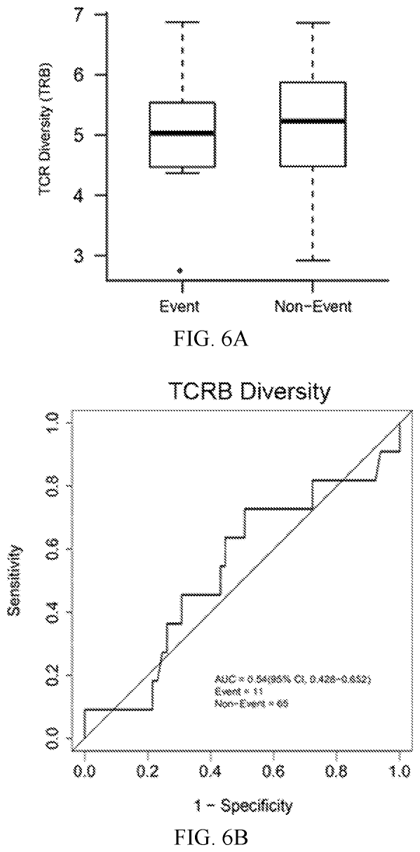

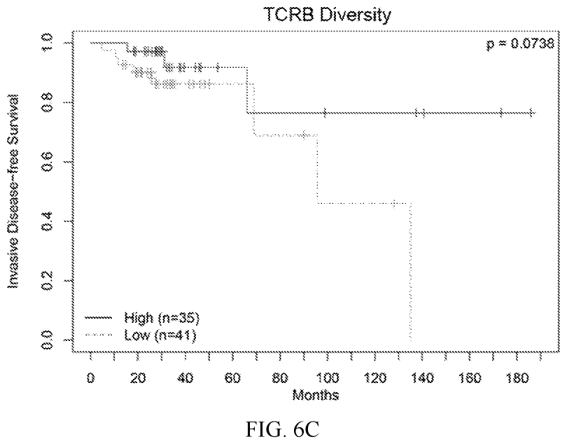

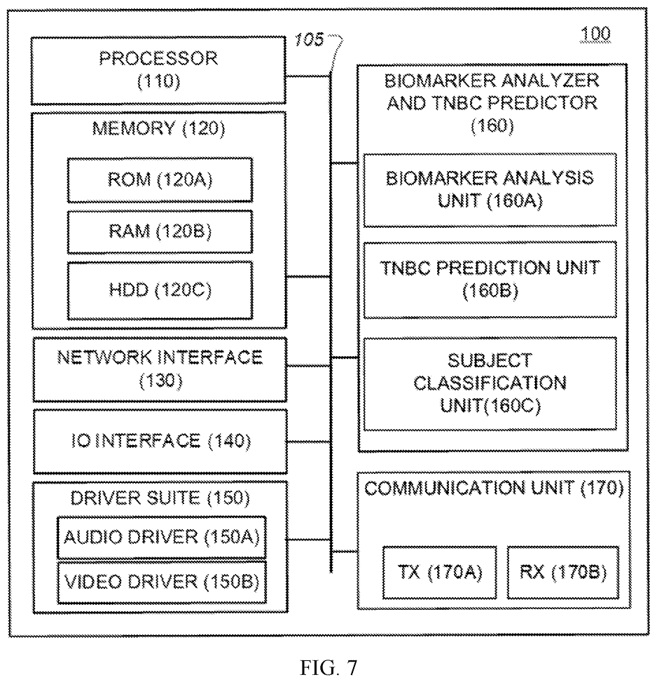

To compare gene signature and other prognostic methods, prognostic values of the PAM 50 call and TCRB diversity were investigated. In the PAM 50 call analysis of the training cohort, 76 patients with TNBC were classified as follows: 31 patients were basal type (40.8%), 7 patients were HER-2 type (9.2%), 22 patients were luminal A type (28.9%), 12 patients were luminal B type (15.8%), and 4 patients were normal type (5.3%). There was no significant difference in terms of iDFS in the KM analysis by type and ROR-S. In the TCRB diversity analysis, with the cut-off that the highest point of the Youden index in ROC analysis (cut-off: 5.26), 35 patients had high diversity of TCRB and the rest of the patients had low diversity of TRB (n=41). However, the diversity of TCRB did not show any significant impact on iDFS (FIGS. 6A-6C).

### Cox Regression Analysis of the Selected Gene Signature

The independency of the selected gene signature (C12orf65; GADD45B; LRBA; LYST; PEX1; PRKAB2; TYW5) was investigated with cox regression analysis. In univariate Cox regression analysis, gene signature was significantly different and positively correlated with prognosis. TNM stage and TRB diversity were not significant but showed a tendency. In multivariable Cox regression analysis with gene signature, TNM stage, and TRB diversity, only the gene signature was statistically significant (Table 4).

TABLE 4
Cox regression analysis of the prognostic gene signature and variables.

| | | | | |
|---|---|---|---|---|
| Variables | n | RC | HR (95% CI) | P-value |

Univariable Cox regression analysis
Candidate Genes

| | | | | | |
|---|---|---|---|---|---|
| C12orf65; GADD45B; LRBA; LYST; | 76 | 4.1459 | 63.17 | (7.95-502.06) | 8.85E−05 |
| PEX1; PRKAB2; TYW5 (low vs high) | | | | | |

Clinicopathological features

| | | | | | |
|---|---|---|---|---|---|
| Age (≤35 years vs >35 years) | 76 | −0.4023 | 0.67 | (0.18-2.47) | 0.546 |
| TNM Stage (1, 2 vs 3) | 76 | 1.0747 | 2.93 | (0.61-14.00) | 0.178 |
| TNM Stage (1 vs 2, 3) | 76 | 1.8319 | 6.25 | (0.79-49.57) | 0.083 |
| PAM50 call ROR-S (Subtype) (Low, Med, High) | 76 | −0.3613 | 0.70 | (0.31-1.55) | 0.377 |
| TCRB Diversity (low vs high) | 76 | −1.1780 | 0.31 | (0.08-1.20) | 0.089 |

Multivariable Cox regression analysis

| | | | | | |
|---|---|---|---|---|---|
| C12orf65; GADD45B; LRBA; LYST; | 76 | 4.4112 | 82.37 | (8.15-832.39) | 1.86E−04 |
| PEX1; PRKAB2; TYW5 (low vs high) | | | | | |
| TNM Stage (1 vs 2, 3) | 76 | 2.4264 | 11.32 | (0.57-223.45) | 0.111 |
| TCRB Diversity (low vs high) | 76 | 0.4175 | 1.52 | (0.33-6.91) | 0.589 |

RC, regression coefficient; HR, hazard ratio; C12orf65, chromosome 12 open reading frame 65; GADD45B, growth arrest and DNA damage-inducible beta; LRBA, LPS-responsive beige-like anchor protein; LYST, lysosomal trafficking regulator; PEX1, peroxisomal biogenesis factor 1; PRKAB2, protein kinase AMP-activated non-catalytic subunit beta 2; TYW5, tRNA-yW synthesizing protein 5; ROR-S, risk of recurrence based on subtype; TRB, T cell receptor beta locus; TNM, tumor-node-metastasis (AJCC stage); HR, hazard ratio; CI, confidence interval.

### Signal Transduction Pathway Analysis and High Interaction Frequency Genes Analysis for Prognostic Gene Signature

Through the biological meta-analysis, gene signature and prognosis-related KEGG signal transduction pathways as well as high interaction frequency genes were found. Transduction pathway analysis revealed that the Epstein-Barr virus infection pathway, pathways in cancer, cell cycle pathway, and viral carcinogenesis pathway were related to the prognostic gene signatures and prognosis. In these pathways, CDK2 and TP53 were the high interaction frequency genes related to gene signatures and prognosis (Table 5).

TABLE 5
Pathways and interacting genes associated with
the gene signature and prognostic features.

| | Name of pathway/Interacting genes |
|---|---|
| Pathways | Epstein-Barr virus infection |
| | Pathways in cancer |
| | Cell cycle |
| | Viral carcinogenesis |
| Interacting genes | CDK2 |
| | TP53 |

CDK2, cyclin dependent kinase 2;
TP53, tumor protein p53

### Example 1: Results for Analysis 2

### Clinical Characteristics of Patients

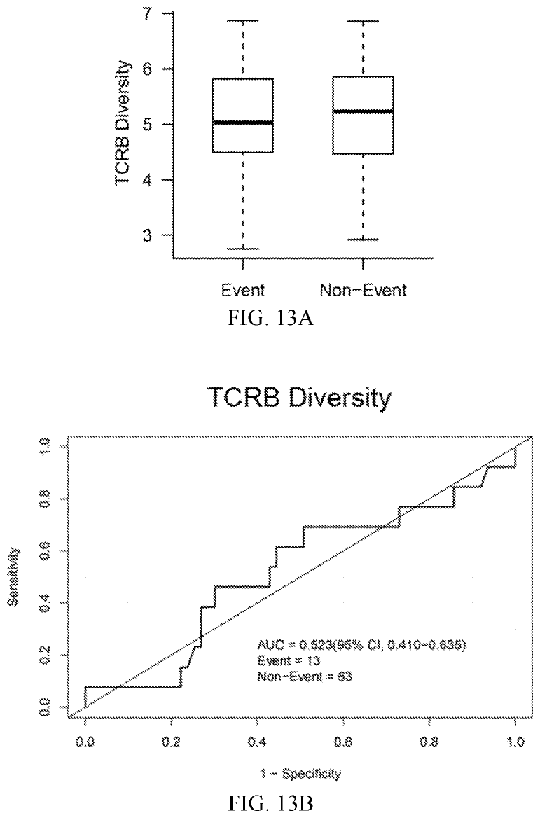

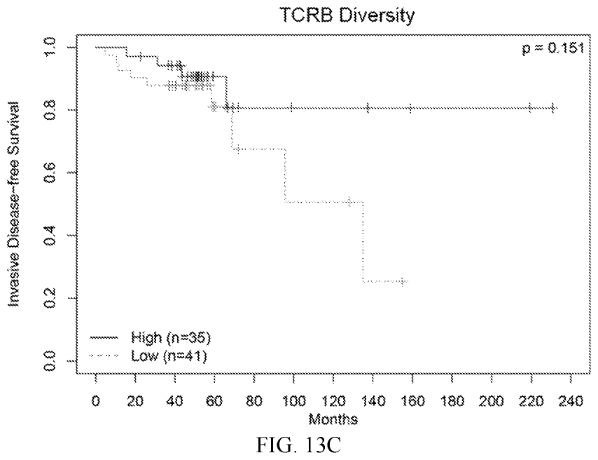

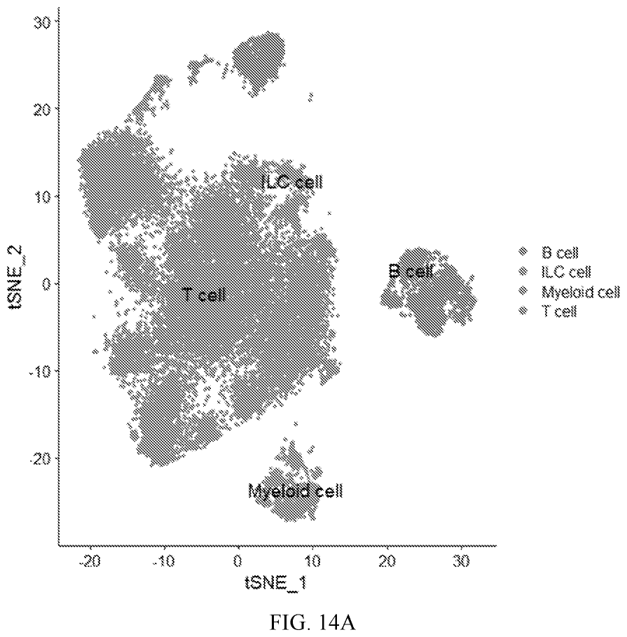

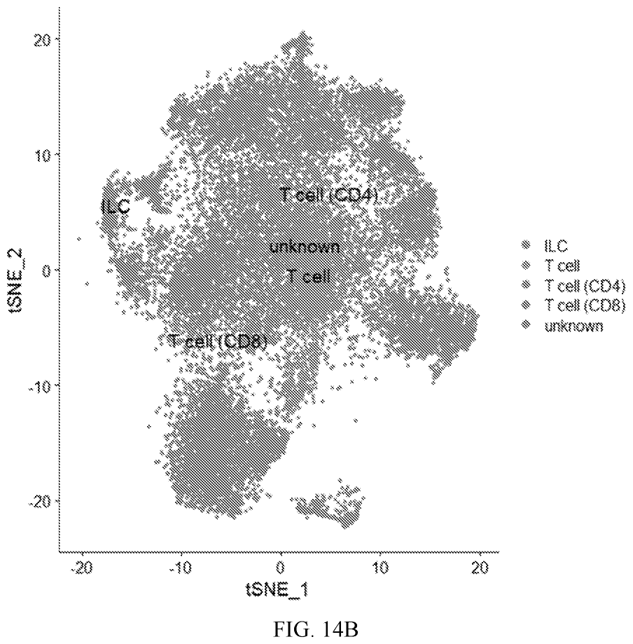

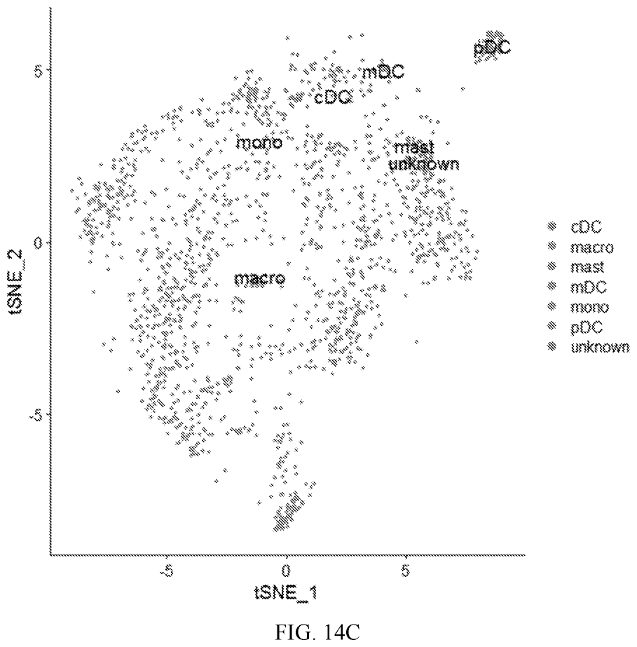

Of the 184 patients, 76 were in the training cohort and 108 in the validation cohort. The clinical characteristics of the patients in the training and validation cohorts are summarized in Table 6. Overall, the patients in the very young age group were not significantly different between the cohorts. Patients in the training cohort were more likely to have earlier-stage diseases than the validation cohort; however, the distribution of stage among the patients who underwent primary surgery or who had residual tumors after neoadjuvant chemotherapy was not different between the two groups. For adjuvant chemotherapy, the TAC (taxotere, adriamycin, and cyclophosphamide) regimen was used more in the validation cohort, probably because there were more advanced stage patients. A schematic representation of the patients and samples is shown in FIGS. 13A-13G.

TABLE 6
Pathological Baselines of the training cohort and validation cohort.

| | Training cohort ( %) | | | Validation cohort (%) | | | Training |
|---|---|---|---|---|---|---|---|
| | | Primary | Residual | | Primary | Residual | vs |
| Pathologic | Total | tumors | tumors | Total | tumors | tumors | validation |
| parameters | (n = 76) | (n = 61) | (n = 15) | (n = 108) | (n = 77) | (n = 31) | p-value* |
| Age (range) | | | | | | | 0.1465 |
| ≤35 years | 17 (22.4) | 10 (16.4) | 7 (46.7) | 36 (33.3) | 24 (31.2) | 12 (38.7) | |
| >35 years | 59 (77.6) | 51 (83.6) | 8 (53.3) | 72 (66.7) | 53 (68.8) | 19 (61.3) | |
| TNM stage | | | | | | | 3.48 × 10⁻⁵ |
| I | 29 (38.2) | 29 (47.5) | — | 18 (16.7) | 18 (23.4) | — | |
| II | 42 (55.3) | 32 (52.5) | 10 (66.7) | 57 (52.8) | 44 (57.1) | 13 (41.9) | |
| III | 5 (6.6) | — | 5 (33.3) | 33 (30.6) | 15 (19.5) | 18 (58.1) | |
| Systemic | | | | | | | — |
| chemotherapy | | | | | | | |
| Neo-adjuvant | 15 (19.7) | — | 15 (100.0) | 73 (67.6) | 42 (54.5) | 31 (100.0) | |
| AC | 2 (13.3) | — | 2 (13.3) | 2 (2.7) | — | 2 (6.5) | |
| AC-D | 6 (40.0) | — | 6 (40.0) | 64 (87.7) | 35 (83.3) | 29 (93.5) | |
| AC-wP | — | — | — | 7 (9.6) | 7 (16.7) | — | |
| AC-PC | 4 (26.7) | — | 4 (26.7) | — | — | — | |
| PCarbo | 2 (13.3) | — | 2 (13.3) | — | — | — | |
| DA | 1 (6.7) | — | 1 (6.7) | — | — | — | |
| Adjuvant | 61 (80.3) | 61 (100.0) | — | 35 (32.4) | 35 (45.5) | — | |
| AC | 6 (9.8) | 6 (9.8) | — | 2 (5.7) | 2 (5.7) | — | |
| TC | 14 (23.0) | 14 (23.0) | — | 1 (2.9) | 1 (2.9) | — | |
| FAC | 14 (23.0) | 14 (23.0) | — | 3 (8.6) | 3 (8.6) | — | |
| AC-D | 7 (11.5) | 7 (11.5) | — | 5 (14.3) | 5 (14.3) | — | |
| AC-wP | 6 (9.8) | 6 (9.8) | — | 4 (11.4) | 4 (11.4) | — | |
| AC-PC | 3 (4.9) | 3 (4.9) | — | — | — | — | |
| TAC | 11 (18.0) | 11 (18.0) | — | 20 (57.1) | 20 (57.1) | — | |
| Event | | | | | | | 0.6052 |
| Presence | 13 (17.1) | 8 (13.1) | 5 (33.3) | 23 (21.3) | 9 (11.7) | 14 (45.2) | |
| Absence | 63 (82.9) | 53 (86.9) | 10 (66.7) | 85 (78.7) | 68 (88.3) | 17 (54.8) | |
| Specimen | | | | | | | |
| Core Biopsy | — | — | — | 42 (38.9) | 42 (54.5) | — | |
| Surgery | 76 (100.0) | 61 (100.0) | 15 (100.0) | 66 (61.1) | 35 (45.5) | 31 (100.0) | |
| Follow-up period, | | | | | | | |
| months | | | | | | | |
| Median | 51.5 | 51.1 | 57.8 | 58.3 | 59.0 | 56.6 | |
| Range | 4.6-230.8 | 11.5-230.8 | 4.6-154.8 | 6.6-99.8 | 12.9-92.7 | 6.6-99.8 | |

TNM, tumor-node-metastasis (AJCC stage); pCR, pathologic complete response; Event, Recurrence or Metastasis; AC, Adriamycin, cyclophosphamide; AC-D, AC followed by docetaxel; TC, Taxotere and cyclophosphamide; FAC, 5-FU, Adriamycin, cyclophosphamide; AC-wP, AC followed by weekly paclitaxel; AC-PC, AC followed by paclitaxel and carboplatin; PCarbo, paclitaxel and carboplatin; DA, docetaxel and adriamycin; TAC, taxotere, and AC.
*p-values were calculated using Fisher's exact test.

### DEG Analyses of Tumor Versus Non-Tumor, and Recurrence and Metastasis Versus Non-Recurrence and Non-Metastasis

In the DEG analysis of tumor versus non-tumor, 9,741 genes were significantly differentially expressed by more than 1.5 fold changes in 10,856 genes. DEG analysis or primary tumors between relapsed and non-relapsed patients revealed 136 out of 10,856 genes showing significant differences in expression by 1.5-fold. Subsequently, out of 10,856 genes, 584 genes were statistically significant in the single Cox analysis. In the DEG analysis in three ways, 59 DEGs overlapped (Table 7).

TABLE 7
DEGS correlated in prognostic of TNBC

| | | | | Event vs | Event vs | |
|---|---|---|---|---|---|---|
| | | NT vs T | NT vs T | non-event | non-event | Cox |
| No | Gene | p-value* | Fold Change | p-value* | Fold Change | p-value |
| 1 | ANKRD36 | 1.74E−03 | 1.84 | 3.25E−02 | 1.83 | 2.96E−03 |
| 2 | ANKRD36BP2 | 6.21E−11 | 18.48 | 3.48E−02 | 2.02 | 9.16E−04 |
| 3 | BSPRY | 1.40E−11 | 17.68 | 1.07E−02 | 2.11 | 9.57E−04 |
| 4 | C12orf65 | 3.02E−12 | 4.32 | 5.00E−05 | −1.67 | 2.37E−03 |
| 5 | C1orf198 | 3.22E−12 | 7.13 | 2.93E−02 | 1.55 | 2.17E−04 |
| 6 | CCDC114 | 8.71E−03 | 1.91 | 1.21E−02 | 2.06 | 2.36E−04 |
| 7 | CLDN4 | 4.35E−10 | 6.70 | 3.94E−03 | 1.89 | 1.48E−02 |
| 8 | CUEDC1 | 2.42E−07 | 2.79 | 1.25E−02 | 1.63 | 1.60E−02 |
| 9 | DCLK2 | 3.36E−05 | 2.12 | 1.30E−02 | 1.78 | 4.08E−04 |
| 10 | DGKH | 1.32E−10 | 8.43 | 5.93E−04 | 2.00 | 2.12E−04 |
| 11 | DIP2B | 2.84E−12 | 6.36 | 5.11E−03 | 1.69 | 3.34E−03 |
| 12 | DISC1 | 2.73E−04 | 2.16 | 2.37E−02 | 1.57 | 2.47E−02 |
| 13 | EMP1 | 6.35E−08 | 3.00 | 1.30E−02 | 1.89 | 1.88E−04 |
| 14 | GADD45B | 4.63E−11 | 6.12 | 1.46E−02 | 1.59 | 6.62E−03 |
| 15 | GLS | 2.84E−12 | 17.00 | 3.77E−03 | 1.57 | 1.44E−02 |
| 16 | GYPC | 1.83E−07 | 2.91 | 3.25E−02 | −1.56 | 3.82E−02 |
| 17 | H2AFX | 2.84E−12 | 34.69 | 9.89E−03 | −1.58 | 4.62E−02 |
| 18 | HRAS | 9.15E−04 | 1.65 | 7.76E−03 | −1.66 | 2.49E−02 |
| 19 | KLF6 | 1.04E−02 | 1.25 | 4.26E−02 | 1.82 | 2.41E−02 |
| 20 | KLF7 | 2.20E−02 | −1.42 | 2.05E−02 | 1.72 | 2.28E−04 |
| 21 | LYST | 2.48E−10 | 3.41 | 2.28E−02 | 1.81 | 1.00E−04 |
| 22 | MALAT1 | 3.48E−10 | −3.29 | 2.64E−02 | 1.80 | 1.94E−03 |
| 23 | MCM3AP.AS1 | 6.58E−11 | 6.76 | 1.30E−02 | 1.64 | 1.34E−03 |
| 24 | MICALL2 | 2.47E−05 | 2.18 | 2.64E−02 | −1.64 | 2.39E−02 |
| 25 | MT2A | 3.22E−12 | 30.27 | 5.11E−03 | 1.78 | 4.21E−04 |
| 26 | NBPF20 | 3.22E−12 | −6.01 | 3.60E−02 | 1.51 | 4.17E−03 |
| 27 | NOTCH2 | 9.75E−12 | 4.35 | 1.83E−02 | 1.95 | 1.17E−04 |
| 28 | NOXA1 | 6.87E−06 | 3.14 | 3.48E−02 | 2.00 | 9.83E−05 |
| 29 | NPR3 | 3.39E−03 | 4.12 | 1.70E−02 | 4.13 | 5.60E−05 |
| 30 | NR6A1 | 6.43E−10 | 4.62 | 3.25E−02 | 1.69 | 1.29E−02 |
| 31 | PAK3 | 1.78E−02 | 2.41 | 2.93E−02 | −2.36 | 4.49E−02 |
| 32 | PARD6B | 5.20E−11 | 7.99 | 2.28E−02 | 1.66 | 1.61E−03 |
| 33 | PEX1 | 2.84E−12 | 9.00 | 2.30E−03 | 1.73 | 4.07E−02 |
| 34 | PRICKLE1 | 7.84E−11 | 6.87 | 5.80E−03 | 1.53 | 2.55E−02 |
| 35 | PYCARD | 2.84E−12 | 13.06 | 3.30E−03 | −1.79 | 2.85E−02 |
| 36 | RASA1 | 3.42E−12 | 4.80 | 4.40E−02 | 1.81 | 6.45E−04 |
| 37 | RASD2 | 4.39E−12 | 24.12 | 2.45E−02 | 2.16 | 5.51E−03 |
| 38 | RASGRP1 | 2.84E−12 | 43.46 | 1.83E−02 | 2.22 | 8.07E−05 |
| 39 | RHPN2 | 2.27E−11 | 13.39 | 2.00E−03 | 1.87 | 2.92E−02 |
| 40 | ROBO1 | 8.91E−09 | 4.12 | 4.85E−02 | −1.76 | 4.27E−02 |
| 41 | RORA | 1.28E−08 | 3.98 | 2.93E−02 | 1.59 | 3.61E−03 |
| 42 | SERTAD4 | 6.58E−11 | 14.86 | 9.13E−03 | 1.91 | 3.45E−03 |
| 43 | SLC22A20 | 1.83E−07 | 4.94 | 3.61E−03 | 2.69 | 1.86E−04 |
| 44 | SLC24A3 | 5.49E−04 | 2.57 | 1.46E−02 | 1.88 | 2.28E−03 |
| 45 | SLC2A12 | 1.66E−10 | 18.89 | 1.90E−02 | 2.55 | 1.40E−02 |
| 46 | SLC39A10 | 8.63E−12 | 7.20 | 3.14E−02 | 1.73 | 4.61E−03 |
| 47 | SLC43A1 | 1.37E−03 | 1.67 | 2.76E−03 | −1.74 | 2.20E−02 |
| 48 | SLC45A4 | 3.06E−11 | 4.30 | 4.69E−03 | 1.77 | 1.16E−04 |
| 49 | SLC6A20 | 8.93E−03 | 2.63 | 3.45E−03 | 2.86 | 1.65E−03 |
| 50 | STAG3L3 | 3.64E−12 | 6.44 | 1.31E−03 | 1.66 | 1.93E−04 |
| 51 | TCTE3 | 2.84E−12 | 109.73 | 2.93E−02 | 1.52 | 1.14E−03 |
| 52 | TMCC2 | 4.11E−11 | 9.92 | 3.02E−03 | 1.98 | 3.95E−04 |
| 53 | TRPS1 | 1.23E−04 | 2.59 | 4.12E−02 | 1.56 | 2.11E−02 |
| 54 | TYW5 | 2.84E−12 | 11.58 | 1.66E−03 | 2.65 | 4.39E−03 |
| 55 | UBE2W | 3.42E−12 | 9.91 | 1.30E−02 | 1.50 | 3.33E−03 |
| 56 | WDSUB1 | 2.84E−12 | 20.06 | 3.77E−03 | 1.56 | 2.46E−03 |
| 57 | WDYHV1 | 2.84E−12 | 15.31 | 5.80E−03 | 1.55 | 1.73E−02 |
| 58 | ZHX2 | 1.32E−11 | 4.29 | 2.45E−02 | 1.63 | 9.63E−03 |
| 59 | ZNF469 | 2.56E−11 | 11.30 | 1.97E−02 | 2.57 | 9.20E−05 |

T, tumor; NT, non-tumor; Event, Recurrence or Metastasis; non-event, non-recurrence and non-metastasis
*p values were calculated using the Wilcoxon test

### Candidate Gene Signatures by Gene Combination and Selected Gene Signature by Cross Validation

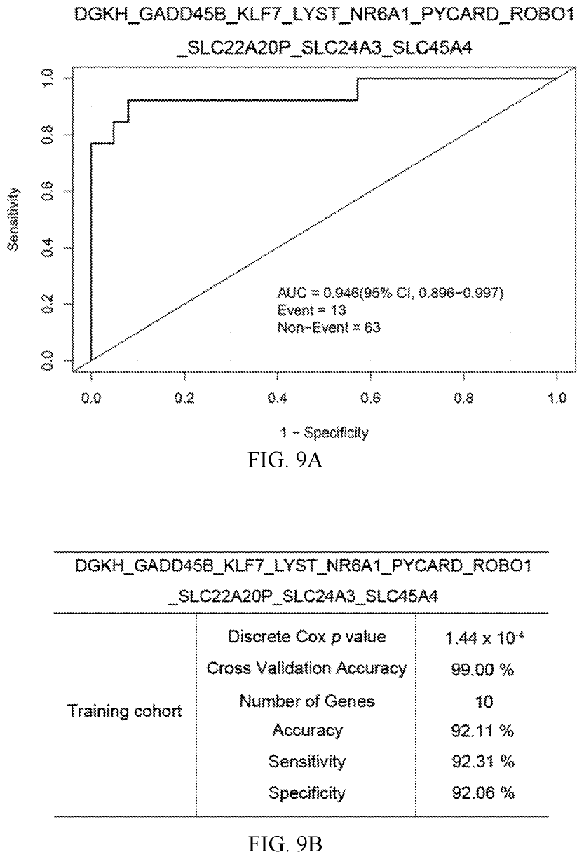

The top 10 candidate gene signatures were ranked with the Continuous Cox p-value. Ten candidates showed values of 80 or higher in sensitivity, specificity, and accuracy. The prognostic gene signature was selected by satisfying statistically in subgroups of cohorts. Also the selected gene signature was DGKH_GADD45B_KLF7_LYST_NR6A1_PYCARD_ROBO1_SLC22A20P_SLC24A3_SLC45A4, showing 99.00% cross-validation accuracy, and it was statistically significant in the discrete Cox analysis. The risk score was calculated with a cut-off value of 5.959715 as follows: (0.818636×DGKH)+(0.018069×GADD45B)+(0.605352×KLF7)+(0.231666×LYST)+(1.305352×NR6A1)+(−0.052086×PYCARD)+(−0.196973×ROBO1)+(0.968759×SLC22A20P)+(0.098331×SLC24A3)+(0.311646×SLC45A4) (FIGS. 9A-9B, Table 8).

TABLE 8
Gene signature candidates as prognostic biomarker of TNBC

| | | No. of | Continuous | | | | |
|---|---|---|---|---|---|---|---|
| | | Combination | Cox | | | | |
| Rank | Gene Signature | genes | p-value | AUC | Sensitivity | Specificity | Accuracy |
| 1 | DGKH; GADD45B; KLF7; | 10 | 1.24E-09 | 0.946 | 92.31 | 92.06 | 92.11 |
| | LYST; NR6A1; PYCARD; | | | | | | |
| | ROBO1; SLC22A20P; | | | | | | |
| | SLC24A3; SLC45A4 | | | | | | |
| 2 | DGKH; DIP2B; EMP1; | 10 | 1.35E-09 | 0.937 | 84.62 | 98.41 | 96.05 |
| | GADD45B; MT2A; | | | | | | |
| | NOTCH2; NR6A1; RORA; | | | | | | |
| | SLC22A20P; SLC24A3 | | | | | | |
| 3 | GADD45B; LYST; NOXA1; | 8 | 2.22E-09 | 0.941 | 84.62 | 96.83 | 94.74 |
| | NR6A1; PYCARD; | | | | | | |
| | SLC22A20P; SLC24A3; | | | | | | |
| | NTAQ1 | | | | | | |
| 4 | DGKH; KLF7; LYST; | 7 | 2.32E-09 | 0.919 | 84.62 | 90.48 | 89.47 |
| | NR6A1; ROBO1; SLC24A3; | | | | | | |
| | SLC6A20 | | | | | | |
| 5 | DGKH; EMP1; GADD45B; | 7 | 2.33E-09 | 0.950 | 92.31 | 88.89 | 89.47 |
| | LYST; SLC22A20P; | | | | | | |
| | SLC24A3; SLC6A20 | | | | | | |
| 6 | CUEDC1; DGKH; EMP1; | 7 | 2.76E-09 | 0.933 | 84.62 | 90.48 | 89.47 |
| | LYST; NOXA1; | | | | | | |
| | SLC22A20P; SLC6A20 | | | | | | |
| 7 | DGKH; KLF7; LYST; | 7 | 2.97E-09 | 0.927 | 92.31 | 92.06 | 92.11 |
| | NR6A1; PRICKLE1; | | | | | | |
| | ROBO1; SLC24A3 | | | | | | |
| 8 | DCLK2; GADD45B; LYST; | 7 | 3.16E-09 | 0.954 | 84.62 | 98.41 | 96.05 |
| | NR6A1; SLC22A20P; | | | | | | |
| | SLC24A3; NTAQ1 | | | | | | |
| 9 | GADD45B; KLF7; LYST; | 7 | 3.33E-09 | 0.918 | 84.62 | 93.65 | 92.11 |
| | NR6A1; ROBO1; | | | | | | |
| | SLC22A20P; SLC6A20 | | | | | | |
| 10 | DCLK2; GADD45B; LYST; | 6 | 5.09E-09 | 0.958 | 84.62 | 95.24 | 93.42 |
| | NR6A1; SLC22A20P; | | | | | | |
| | NTAQ1 | | | | | | |

AUC, Area under the curve; DGKH, diacylglycerol kinase eta; GADD45B, Growth arrest and DNA damage inducible beta; KLF7, Kruppel like factor 7; LYST, lysosomal trafficking regulator; NR6A1, nuclear receptor subfamily 6 group A member 1; PYCARD, PYD and CARD domain containing; ROBO1, roundabout guidance receptor 1; SLC22A20P, solute carrier family 22 member 20, pseudogene; SLC24A3, solute carrier family 24 member 3; SLC45A4, solute carrier family 45 member 4; DIP2B, disco interacting protein 2 homolog B; EMP1, epithelial membrane protein 1; MT2A, metallothionein 2A; NOTCH2, notch receptor 2; RORA, RAR related orphan receptor A; NOXA1, NADPH oxidase activator 1; NTAQ1, N-terminal glutamine amidase 1; SLC6A20, solute carrier family 6 member 20; CUEDC1, CUE domain containing 1; PRICKLE1, prickle planar cell polarity protein 1; DCLK2, doublecortin like kinase 2

### Prognostic Significance of Gene Signature in the Training Cohort

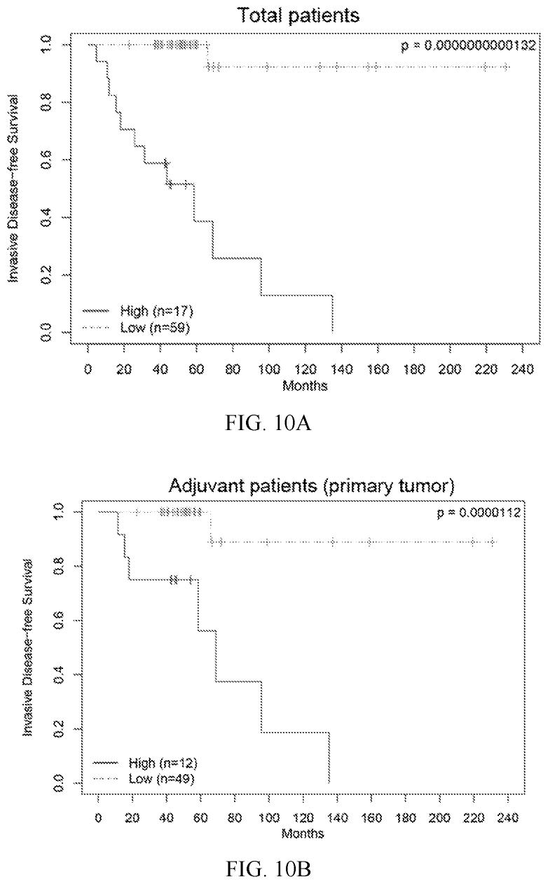

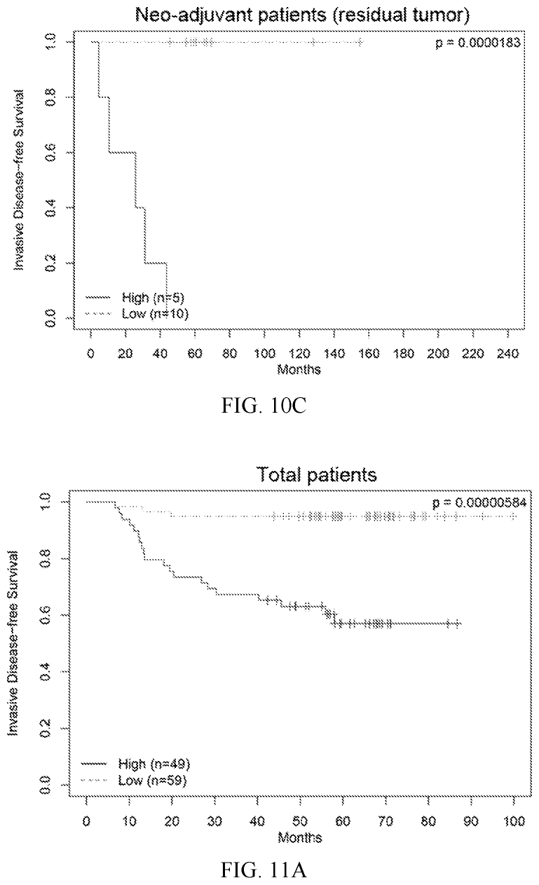

During the median follow-up of 51.5 months (range: 4.6-230.8), patients with tumors with high-risk gene signatures (n=17) showed significantly shorter iDFS (median, 95% confidence interval [CI]; 58.5, 25.8—not reached) than those with low-risk signatures (n=59, median not reached, p=1.32×10⁻¹¹) in the overall population (FIG. 10A). Further analysis in the separate group of patients who underwent primary surgery and in the patients with residual tumors after neoadjuvant chemotherapy showed similar results. Among patients who received primary surgery and patients with residual tumors after neoadjuvant chemotherapy, the median iDFS in the high-risk group was 68.9 months (95% CI: 58.5—not reached; p=0.0000112) and 25.8 months (95% CI: 10.6—not reached, p=0.0000183), respectively, and the median iDFS in the low-risk group did not reach the median (FIGS. 10B, 10C).

### Prognostic Significance of Gene Signatures in the Validation Cohort

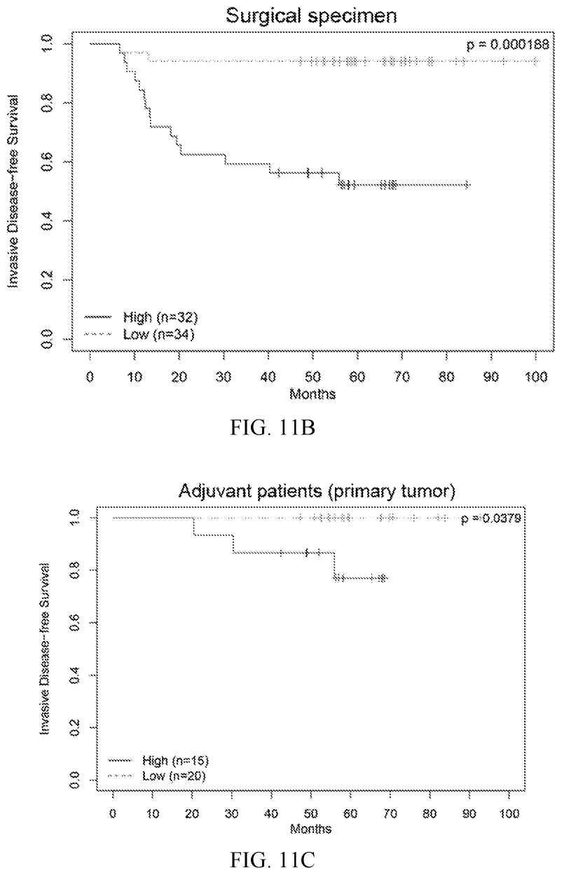

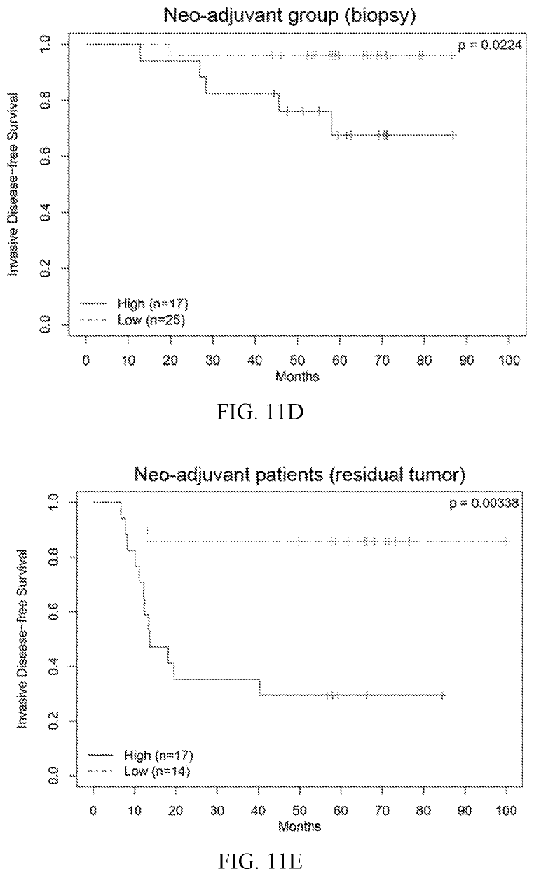

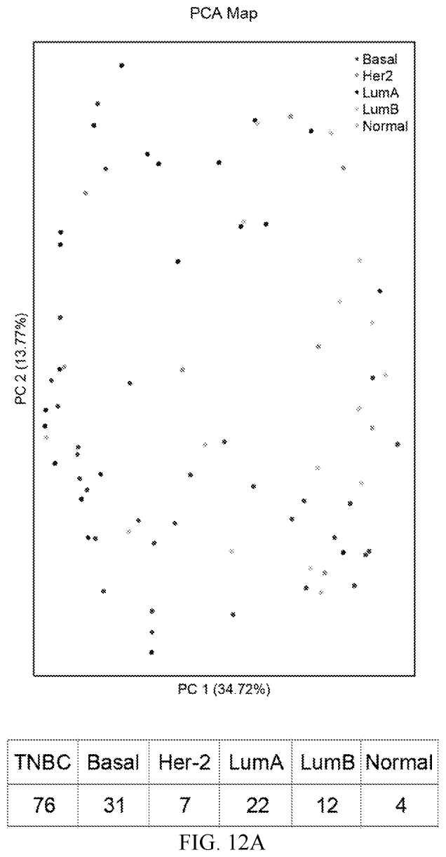

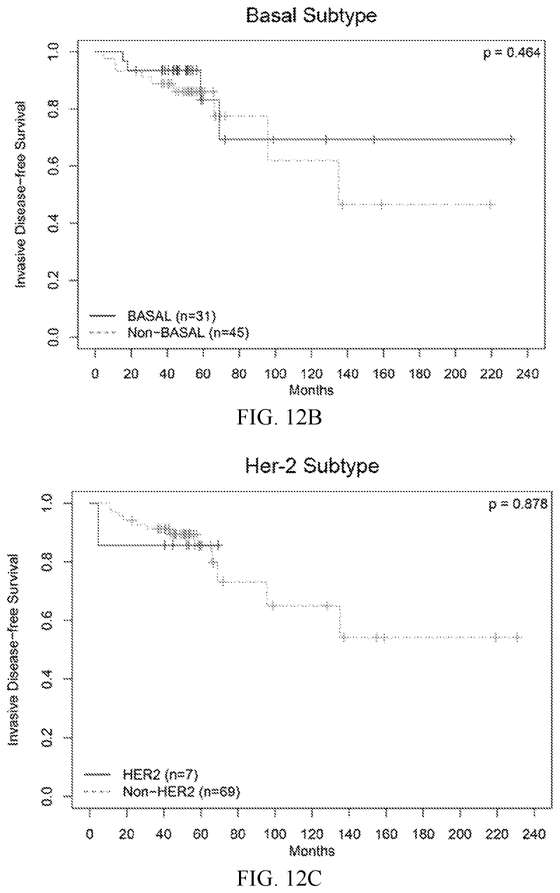

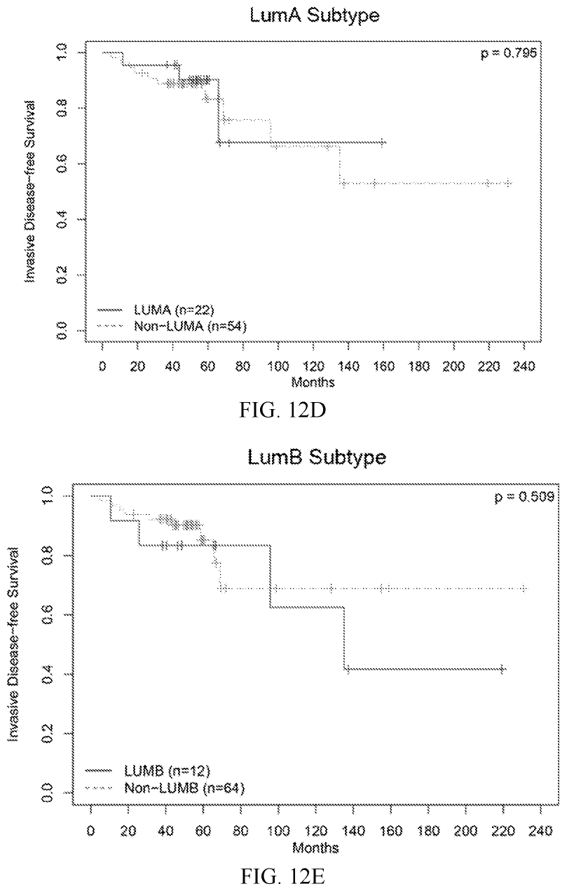

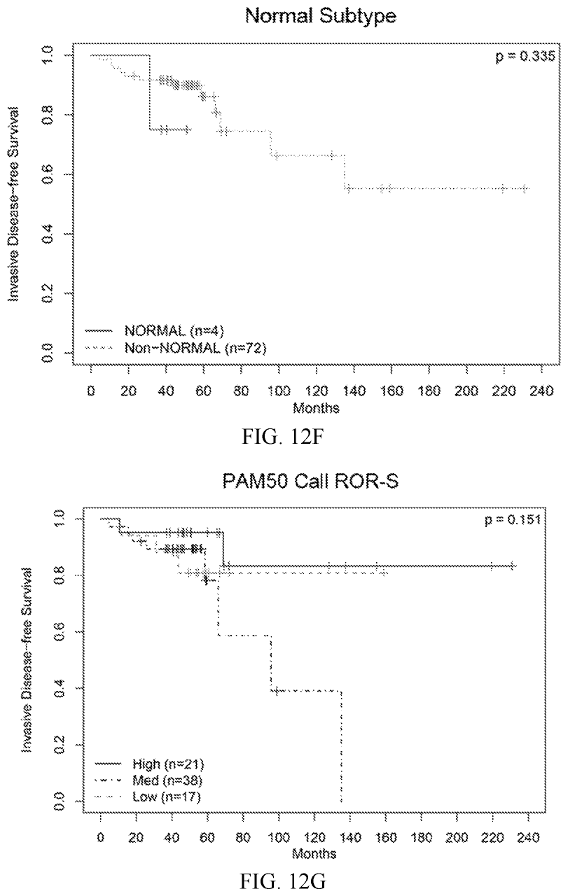

Median follow up time for validation cohort was 58.3 months (range; 6.6-99.8). In the overall validation cohort, although the median iDFS of the patients with high-risk genetic signatures was not reached, there was a significantly higher risk for recurrence or metastasis than patients with low-risk gene signatures (median iDFS not reached and p=0.00000584 in log-rank test). When the patients were sub-divided according to the treatment sequence, the prognostic significance of the gene signatures in the surgical tissues from the patients who underwent primary surgery was consistent with the training cohort (p=0.0379); however, the median iDFS in the high-risk and low-risk groups was not reached yet. High-risk gene signatures were still valid in predicting the prognosis of patients with residual tumors after neoadjuvant chemotherapy; median iDFS in the high-risk group was 13.6 months (95% CI, 12.2—not reached), but it was not reached in the low-risk group (p=0.00338). Also, when the gene signatures were examined in the tissues obtained by core biopsy in the neoadjuvant chemotherapy group, prognostic significance in iDFS was statistically significant (p=0.0224). (FIGS. 11A-11E).

### Investigation of Other Potential Prognostic Factors

To compare gene signatures and other prognostic methods, prognostic values of the PAM 50 call and TCRB diversity were investigated. In the PAM 50 call analysis of the training cohort, 76 patients with TNBC were classified as follows: 31 patients were basal type (40.8%), 7 patients were HER-2 type (9.2%), 22 patients were luminal A type (28.9%), 12 patients were luminal B type (15.8%), and 4 patients were normal type (5.3%). There was no significant difference in terms of iDFS in the KM analysis by type and ROR-S. In the TCRB diversity analysis, with the cut-off that the highest point of the Youden index in ROC analysis (cut-off: 5.26), 35 patients had high diversity of TCRB and the rest of the patients had low diversity of TRB (n=41). However, the diversity of TCRB did not show any significant impact on iDFS (FIGS. 13A-13C).

### Cox Regression Analysis of the Selected Gene Signature

The independency of the selected gene signature (DGKH_GADD45B_KLF7_LYST_NR6A1_PYCARD_ROBO1_SLC22A20P_SLC24A3_SLC45A4) was investigated with cox regression analysis. In univariate Cox regression analysis, gene signature was significantly different and positively correlated with prognosis. TNM stage was not significant but showed a tendency. In multivariable Cox regression analysis with gene signature, TNM stage, and TRB diversity, only the gene signature was statistically significant (Table 9).

TABLE 9
Cox regression analysis of the prognostic gene signature and variables.

| | | | | |
|---|---|---|---|---|
| Variables | n | RC | HR (95% CI) | P-value |

Univariable Cox regression analysis
Candidate Genes

| | | | | | |
|---|---|---|---|---|---|
| DGKH; GADD45B; KLF7; LYST; NR6A1; | 76 | 3.9606 | 52.49 | (6.81-404.53) | 1.44E−04 |
| PYCARD; ROBO1; SLC22A20P; SLC24A3; | | | | | |
| SLC45A4 (low vs high) | | | | | |

Clinicopathological features

| | | | | | |
|---|---|---|---|---|---|
| Age (≤35 years vs >35 years) | 76 | −0.1605 | 0.85 | (0.25-2.94) | 0.799 |
| TNM Stage (1, 2 vs 3) | 76 | 0.9281 | 2.53 | (0.55-11.74) | 0.236 |
| TNM Stage (1 vs 2, 3) | 76 | 1.9985 | 7.38 | (0.95-57.08) | 0.056 |
| PAM50 call ROR-S (Subtype) (Low, Med, | 76 | −0.4875 | 0.61 | (0.30-1.27) | 0.186 |
| High) | | | | | |
| TCRB Diversity (low vs high) | 76 | −0.8453 | 0.43 | (0.13-1.41) | 0.162 |

Multivariable Cox regression analysis

| | | | | | |
|---|---|---|---|---|---|
| DGKH; GADD45B; KLF7; LYST; NR6A1; | 76 | 4.2879 | 72.81 | (7.04-752.75) | 3.21E−04 |
| PYCARD; ROBO1; SLC22A20P; SLC24A3; | | | | | |
| SLC45A4 (low vs high) | | | | | |
| TNM Stage (1 vs 2, 3) | 76 | 2.3537 | 10.52 | (0.67-165.09) | 0.094 |
| TCRB Diversity (low vs high) | 76 | 0.3597 | 1.43 | (0.37-5.56) | 0.603 |

RC, Regression coefficient;
HR, hazard ratio;
CI, confidence interval;
DGKH, diacylglycerol kinase eta;
GADD45B, Growth arrest and DNA damage inducible beta;
KLF7, Kruppel like factor 7;
LYST, lysosomal trafficking regulator;
NR6A1, nuclear receptor subfamily 6 group A member 1;
PYCARD, PYD and CARD domain containing;
ROBO1, roundabout guidance receptor 1;
SLC22A20P, solute carrier family 22 member 20, pseudogene;
SLC24A3, solute carrier family 24 member 3;
SLC45A4, solute carrier family 45 member 4;
TNM, Tumor-Node-Metastasis (AJCC stage);
ROR-S, risk of recurrence based on subtype;
TCRB, T cell receptor beta locus

### Signal Transduction Pathway Analysis and High Interaction Frequency Genes Analysis for Prognostic Gene Signature

Through the biological meta-analysis, gene signature and prognosis-related KEGG signal transduction pathways as well as high interaction frequency genes were found. Transduction pathway analysis revealed that the pathways in cancer, PI3K-Akt signaling pathway, Alzheimer disease pathway, Human cytomegalovirus infection pathway, Hepatitis C pathway, Breast cancer pathway and MAPK signaling pathway were related to the prognostic gene signatures and prognosis. In these pathways, KRAS, HRAS and APP were the high interaction frequency genes related to gene signatures and prognosis (Table 10).

TABLE 10
Pathways and interacting genes associated with
the gene signature and prognostic features.

| | Name of pathway/Interacting genes |
|---|---|
| Pathways | Pathways in cancer |
| | PI3K-Akt signaling pathway |
| | Alzheimer disease |
| | Human cytomegalovirus infection |
| | Hepatitis C |
| | Breast cancer |
| | MAPK signaling pathway |
| Interacting genes | KRAS |
| | HRAS |
| | APP |

KRAS, KRAS proto-oncogene, GTPase;
HRAS, HRAS proto-oncogene, GTPase;
APP, amyloid beta precursor protein

### Example 2: Prognostic Gene Signature Reflecting CD8+ T Cell Enrichment in Early Stage-Triple Negative Breast Cancer

Method: 76 patients with TNBC were enrolled for gene expression profiles and GSE 169246 data were collected for single cell RNA (scRNA) profiles. Median Follow-up period of enrolled patients was 51.5 months (range: 4.6-230.8). Of the enrolled patients, 13 patients had recurrence or metastasis. Using the HiSeq 4000 sequencer, RNA sequencing was conducted to analyze the gene expression profiles of tumor samples from TNBC patients. Single cell RNA analysis was performed using Seurat package (v.4.0.5). Differentially expressed genes (DEGs) were defined as satisfying both conditions that satisfying in Cox regression analysis and Wilcoxon analysis in gene expression profiles, and that satisfying in logistic regression analysis in CD8+t cell profiles of scRNA analysis. Gene signature was analyzed by combination of above DEGs. Gene signature was marking on t-SNE of scRNA profiles. Statistical analyses were conducted using R language (v.3.4.3).

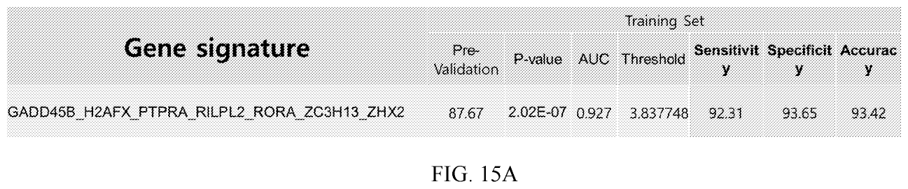

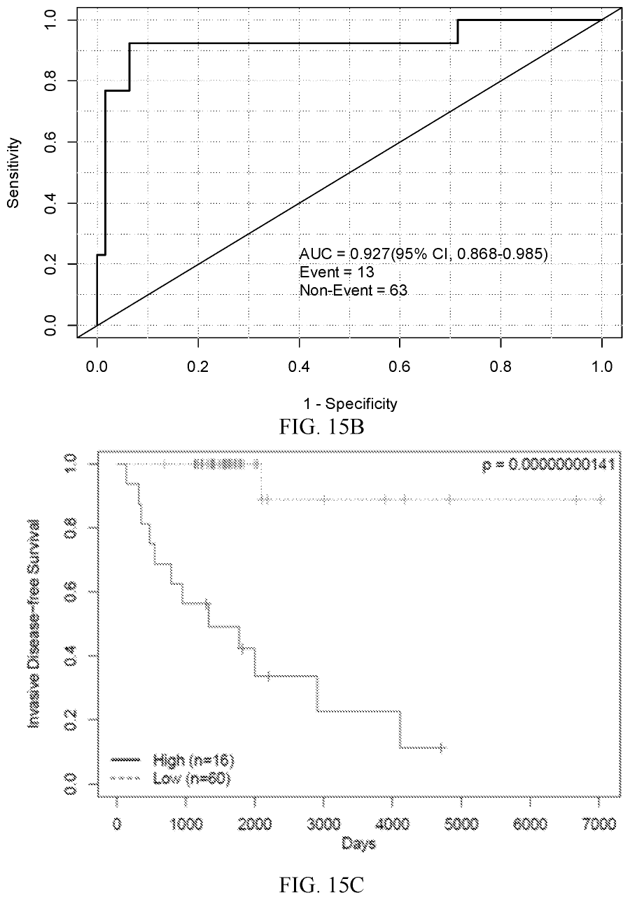

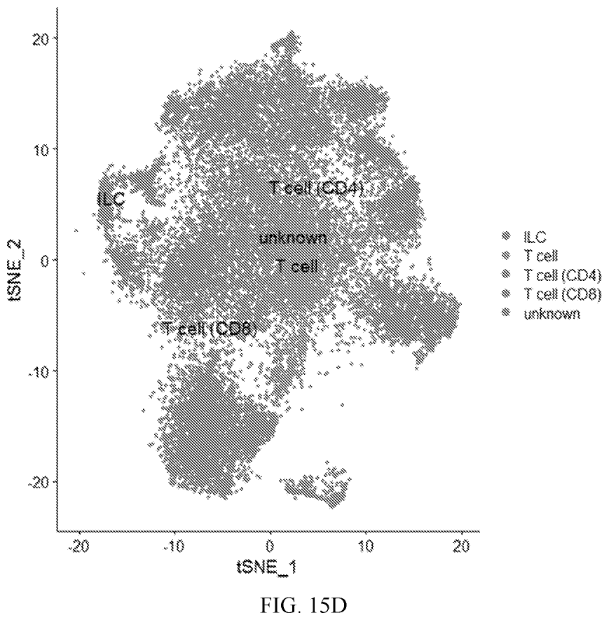

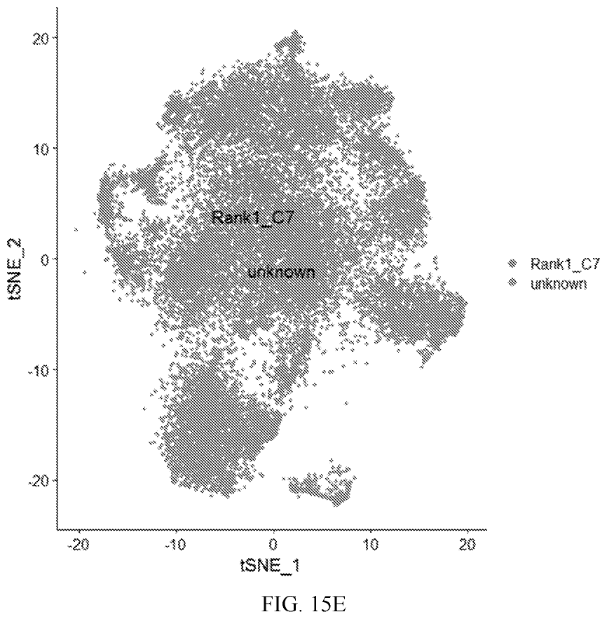

Results: A gene signature reflecting CD8+ T cell enriched feature that stratified patients with TNBC by risk score was identified. Gene-set signatures related to CD8+ T cells were identified as following: GADD45B, H2AFX, PTPRA, RILPL2, RORA, ZC3H13, ZHX2 (sensitivity=92.31%; specificity=93.65%; accuracy=93.42%). In Kaplan Meier (KM) analysis, patients with tumors with high-risk gene signatures (n=16, median iDFS=42.7) showed significantly shorter iDFS than those with low-risk gene signatures (n=60, median iDFS not reached) (FIGS. 15A-15B). CD8+t cells related gene signature was marked on CD8+ T cell near the CD4+ T cell in t-SNE (FIGS. 15C-15E).

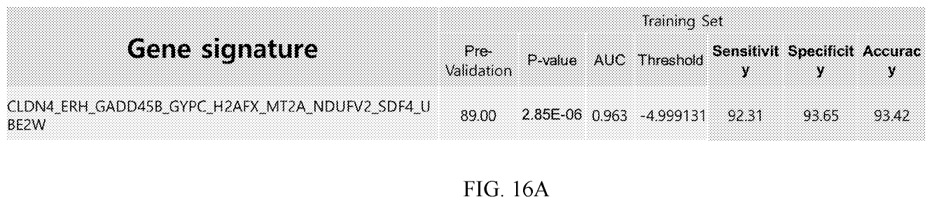

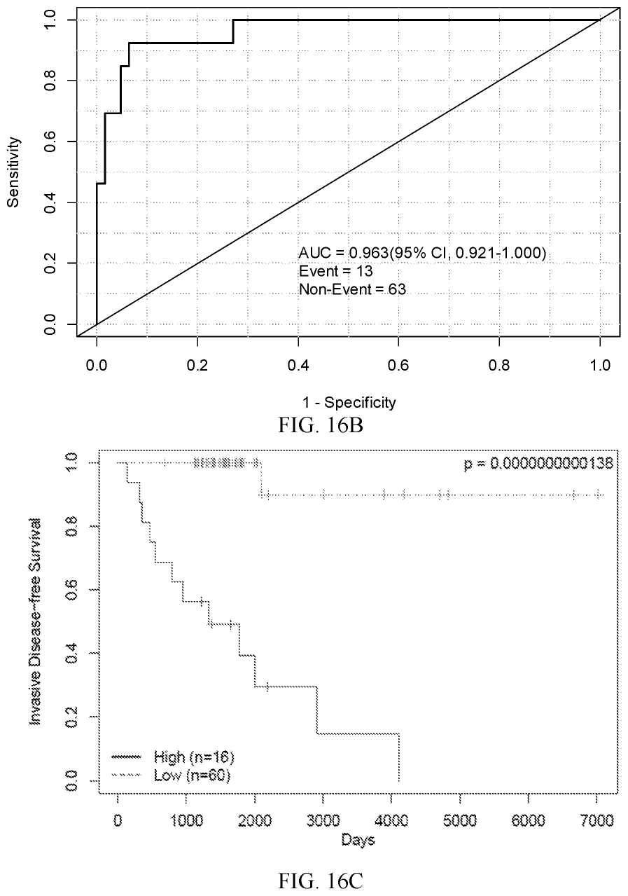

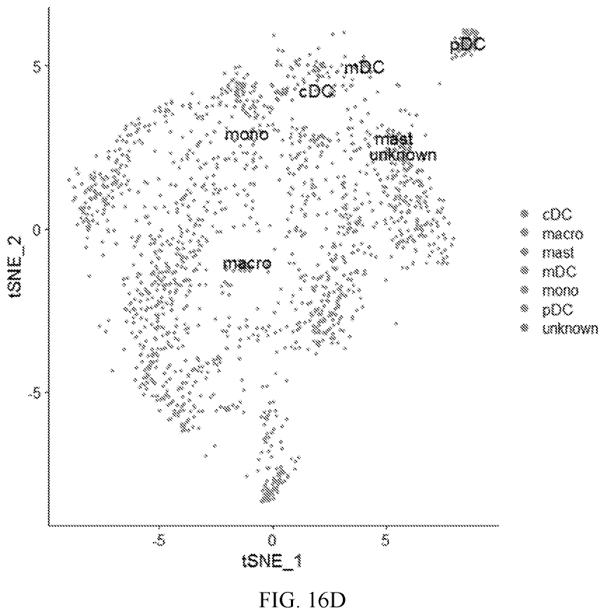

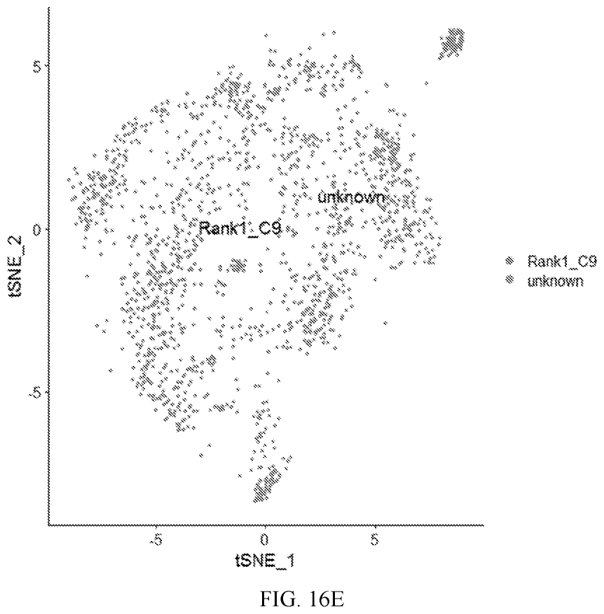

Additionally, a gene signature reflecting macrophage enriched feature that stratified patients with TNBC by risk score was identified. Gene-set signatures related to macrophages were identified as following: CLDN4, ERH, GADD45B, GYPC, H2AFX, MT2A, NDUFV2, SDF4, UBE2 W (AUC=0.963, sensitivity=92.31%; specificity=93.65%; accuracy=93.42%) In Kaplan Meier (KM) analysis, patients with tumors with high-risk gene signatures (n=16, median iDFS=42.7 months) showed significantly shorter iDFS than those with low-risk gene signatures (n=60, median iDFS not reached) (FIGS. 16A-16B). Macrophages related gene signature was marked on macrophage, monocyte and dendritic cells in t-SNE (FIGS. 16C-16E).

Non-Limiting Embodiment of System and Computer-Implemented Method for Predicting Prognosis of, and/or a Treatment Outcome for, a Subject Diagnosed with TNBC, or an at Risk-Candidate of Developing TNBC

FIG. 7 shows a non-limiting embodiment of a system 100 that is constructed according to the principles of the invention. The system 100 is configured to receive computer-readable data of a plurality of biomarkers (including, for example, a panel of biomarkers) for a sample from a subject, which can be obtained as described with respect to certain embodiments provided in this disclosure, and predict a prognosis of, and/or a treatment outcome for, a subject diagnosed with cancer (e.g., breast cancer, such as a TNBC), or an at risk-candidate of developing cancer (e.g., breast cancer, such as a TNBC). In certain embodiments, the plurality of biomarkers include at least two biomarkers, at least three biomarkers, at least four biomarkers, at least five biomarkers, at least six biomarkers, or seven biomarkers selected from the group consisting of ankyrin repeat domain 36 (ANKRD36), ankyrin repeat domain 36B pseudogene 2 (ANKRD36BP2), B-box and SPRY domain-containing (BSPRY), chromosome 12 open reading frame 65 (C12orf65), chromosome 2 open reading frame 49 (C2orf49), chromosome 1 open reading frame 198 (C1orf198), coiled-coil domain containing 114 (CCDC114), claudin 4 (CLDN4), CUE domain containing 1 (CUEDC1), outer dynein arm docking complex subunit 1 (ODAD1), cell growth regulator with EF-hand domain 1 (CGREF1), DEP domain containing 7 (DEPDC7), doublecortin like kinase 2 (DCLK2), diacylglycerol kinase (DGKH), disco interacting protein 2 homolog B (DIP2B), disrupted-in-schizophrenia 1 (DISC1), epithelial membrane protein 1 (EMP1), ERH mRNA splicing and mitosis factor (ERH), growth arrest and DNA damage inducible beta (GADD45B), glutaminase (GLS), grainyhead like transcription factor 1 (GRHL1), glycophorin C (GYPC), H2A histone family member X (H2AFX), HRas proto-oncogene (HRAS), intracellular adhesion molecule 1 (ICAM1), interphotoreceptor matrix proteoglycan 2 (IMPG2), potassium voltage-gated channel subfamily C member 3 (KCNC3), kruppel like factor 6 (KLF6), kruppel like factor 7 (KLF7), keratin 17 (KRT17), LON peptidase N-terminal domain and RING finger protein 2 (LONRF2), LPS responsive beige-like anchor protein (LRBA), leucine rich repeat, Ig-like and transmembrane domains 3 (LRIT3), leucine rich repeat containing 37B (LRRC37B), LSM11, U7 small nuclear RNA associated (LSM11), lysosomal trafficking regulator (LYST), metastasis associated lung adenocarcinoma transcript 1 (MALAT1), minichromosome maintenance complex component 3 associated protein antisense RNA 1 (MCM3AP_AS1), MICAL like 2 (MICALL2), MHC class I polypeptide-related sequence B (MICB), metallothionein 2A (MT2A), myelin expression factor 2 (MYEF2), NEDD4 binding protein 3 (N4BP3), neuroblastoma breakpoint family member 20 (NBPF20), NADH: Ubiquinone Oxidoreductase Core Subunit V2 (NDUFV2), neurogenic locus notch homolog protein 2 (NOTCH2), NADPH oxidase activator 1 (NOXA1), natriuretic peptide receptor 3 (NPR3), nuclear receptor subfamily 6 group A member 1 (NR6A1), P21 (RAC1) activated kinase 3 (PAK3), pantothenate kinase 3 (PANK3), par-6 family cell polarity regulator beta (PARD6B), peroxisomal biogenesis factor 1 (PEX1), piggyBac transposable element derived 4 (PGBD4), praja ring finger ubiquitin ligase 1 (PJA1), pleckstrin homology and FYVE domain containing 1 (PLEKHF1), purine nucleoside phosphorylase (PNP), protein tyrosine phosphatase receptor type A (PTPRA), protein phosphatase, Mg2+/Mn2+ dependent 1K (PPM1K), prickle planar cell polarity protein 1 (PRICKLE1), protein kinase AMP-activated non-catalytic subunit beta 2 (PRKAB2), PYD and CARD domain containing (PYCARD), RAS p21 protein activator 1 (RASA1), RASD family member 2 (RASD2), RAS guanyl releasing protein 1 (RASGRP1), rhophilin RHO GTPase binding protein 2 (RHPN2), Rab interacting lysosomal protein like 2 (RILPL2), roundabout guidance receptor 1 (ROBO1), RAR related orphan receptor A (RORA), Stromal cell derived factor 4 (SDF4), SERTA domain containing 4 (SERTAD4), shisa family member 5 (SHISA5), signal induced proliferation associated 1 like 2 (SIPA1L2), solute carrier family 22 member 20 (SLC22A20P), solute carrier family 24 member 3 (SLC24A3), solute carrier family 2 member 12 (SLC2A12), solute carrier family 39 member 10 (SLC39A10), solute carrier family 25 member 40 (SLC25A40), solute carrier family 43 member 1 (SLC43A1), solute carrier family 45 member 4 (SLC45A4), solute carrier family 6 member 20 (SLC6A20), spectrin beta, erthrocytic (SPTB), stromal antigen 3-like 3 (STAG3L3), sushi domain containing 3 (SUSD3), TATA-Box binding protein associated factor 10 (TAF10), t-complex-associated testis expressed 3 (TCTE3), transmembrane channel like 7 (TMC7), transmembrane and coiled-coil domain family 2 (TMCC2), transcriptional repressor GATA binding 1 (TRPS1), tubulin tyrosine ligase like 4 (TTLL4), tRNA-yW synthesizing protein 5 (TYW5), ubiquitin conjugating enzyme E2 W (UBE2 W), WD repeat, sterile alpha motif and U-box domain containing 1 (WDSUB1), N-protein N-terminal glutamine amidohydrolase (WDYHV1), N-terminal glutamine amidase 1 (NTAQ1), zinc finger BED-type containing 6 (ZBED6), zinc finger and BTB domain containing 46 (ZBTB46), zinc finger CCCH-type containing 13 (ZC3H13), zinc fingers and homeoboxes 2 (ZHX2), zinc finger protein 217 (ZNF217), zinc finger protein 233 (ZNF233), zinc finger protein 248 (ZNF248), zinc finger protein 469 (ZNF469), and zinc finger protein 785 (ZNF785). In certain embodiments, the system 100 is configured to calculate the plurality of biomarkers using RNA sequencing data from tumor tissue of triple negative breast cancer patients. In certain embodiments, the system 100 is configured to determine a score based on the plurality of biomarkers (for example, 6, 7, 8, 9, or 10 gene signatures-(a) DGKH, GADD45B, KLF7, LYST, NR6A1, PYCARD, ROBO1, SLC22A20P, SLC24A3, and SLC45A4; (b) DGKH, DIP2B, EMP1, GADD45B, MT2A, NOTCH2, NR6A1, RORA, SLC22A20P, and SL24A3; (c) GADD45B, LYST, NOXA1, NR6A1, PYCARD, SLC22A20P, SLC24A3, and NTAQ1; (d) DGKH, KLF7, LYST, NR6A1, ROBO1, SLC24A3, and SLC6A20; (e) DGKH, EMP1, GADD45B, LYST, SLC22A20P, SLC24A3, and SLC6A20; (f) CUEDC1, DGKH, EMP1, LYST, NOXA1, SLC22A20P, and SLC6A20; (g) DGKH, KLF7, LYST, NR6A1, PRICKLE1, ROBO1, and SLC24A3; (h) DCLK2, GADD45B, LYST, NR6A1, SLC22A20P, SLC24A3, and NTAQ1; (i) GADD45B, KLF7, LYST, NR6A1, ROBO1, SLC22A20P, and SLC6A20; (j) DCLK2, GADD45B, LYST, NR6A1, SLC22A20P, and NTAQ1; (k) C12orf65, GADD45B, LRBA, LYST, PEX1, PRKAB2, and TYW5; (l) LYST, PEX1, ODAD1, DEPDC7, MICALL2, SLC43A1, and SLC6A20; (m) C12orf65, GADD45B, LYST, PEX1, RASA1, SLC45A4, and NTAQ1; (n) TYW5, DEPDC7, SLC43A1, CGREF1, MICB, HRAS, and MT2A; (o) LRBA, LYST, PEX1, DEPDC7, SLC43A1, MICB, and C2orf49; (p) GADD45B, PEX1, DEPDC7, SLC43A1, LSM11, and PJA1; (q) DEPDC7, SLC43A1, MICB, C2orf49, HRAS, KCNC3, and MT2A; (r) LYST, DEPDC7, SLC43A1, SLC6A20, MICB, and HRAS; (s) PEX1, MICALL2, SLC43A1, RASA1, KCNC3, and LRIT3; (t) GADD45B, SLC43A1, SLC45A4, KCNC3, SHISA5, and SLC25A40; (u) GADD45B, H2AFX, PTPRA, RILPL2, RORA, ZC3H13, and ZHX2; or (v) CLDN4, ERH, GADD45B, GYPC, H2AFX, MT2A, NDUFV2, SDF4, and UBE2 W) and predict and classify cancer (e.g., TNBC) patients to high or low risk groups for disease progression, relapse, recurrence, and/or death. In an embodiment, the system 100 is configured to analyze a plurality of biomarkers, predict prognosis, and provide a guide for a precise treatment strategy.

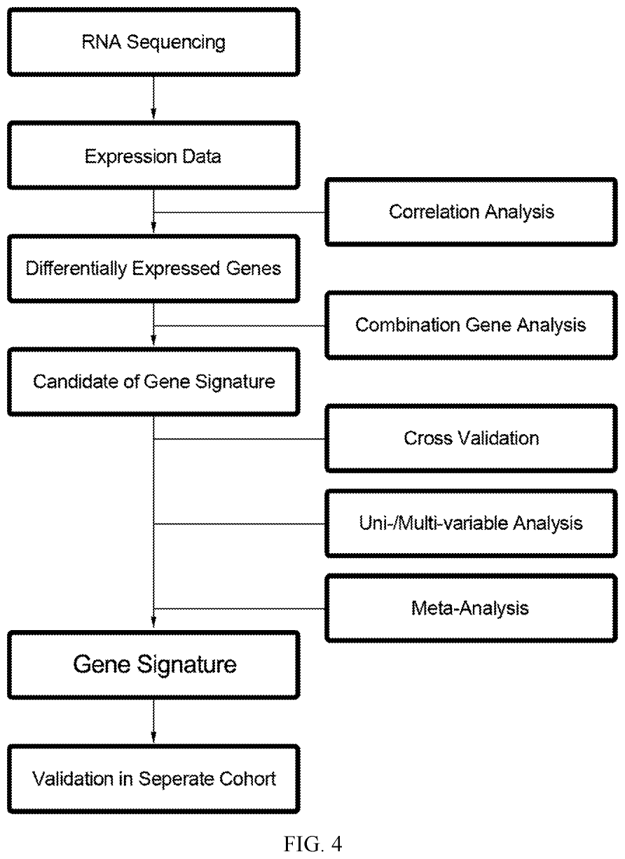

The system 100 comprises a processor 110, a memory 120, a network interface 130, an input-output (IO) interface 140, a driver suite 150, a biomarker analyzer and cancer (e.g., TNBC) predictor 160, and a communication unit 170, all of which can be arranged to connect to a bus 105. In a non-limiting embodiment, the system 100 is configured to perform the process described in FIG. 4. The system 100 can include a machine learning platform containing supervised machine learning, unsupervised machine learning or a combination of supervised and unsupervised machine learning that can perform one or more machine learning processes.

In an embodiment, the system 100 is configured to perform combination gene analysis and predict an n-gene signature of optimal genes that can be used as biomarkers in large-scale analysis, where n is a positive non-zero integer. The system 100 can be configured to perform pre-validation (for example, a 6, 7, 8, 9, or 10-signature cross-validation, where n=6, 7, 8, 9, or 10) by a machine learning process, and, then preform a validation, such as, for example, a 10-gene signature validation in a separate validation cohort. The system 100 can be configured to perform a meta-analysis, by a machine learning process, of the n-gene (for example, 10-gene) to determine or confirm biological relevance between the n-gene signature and cancer (e.g., TNBC). The system 100 can be configured to update parametric model values of the machine learning platform during operation.

In a non-limiting application of the system 100, patients with early TNBC classified to high risk for prognosis and/or treatment outcome by the system 100 can be candidates for further systemic treatment, for example, in addition to standard care.

In an embodiment, the system 100 can be utilized as a tool to select patients for escalation or de-escalation trial. In this regard, patients can benefit from risk-based care, and not be subject to a one-size-fits-all treatment.

The biomarker analyzer and cancer (e.g., TNBC) predictor 160 can include a computing device, or be included in a computing device. The cancer (e.g., TNBC) biomarker analyzer and cancer (e.g., TNBC) predictor 160 can include a machine learning platform containing supervised machine learning, unsupervised machine learning or a combination of supervised and unsupervised machine learning. The machine learning platform can include, for example, an artificial neural network (ANN), a convolutional neural network (CNN), a temporal convolutional network (TCN), a deep CNN (DCNN), an RCNN, a Mask-RCNN, a deep convolutional encoder-decoder (DCED), a recurrent neural network (RNN), a neural Turing machine (NTM), a differential neural computer (DNC), a support vector machine (SVM), a deep learning neural network (DLNN), a long short-term memory (LSTM), Naïve Bayes, decision trees, linear regression, Q-learning, temporal difference (TD), deep adversarial networks, fuzzy logic, or any other machine intelligence platform capable of supervised or unsupervised machine learning. The machine learning platform can include a machine learning model. The biomarker analyzer and cancer (e.g., TNBC) predictor 160 can include a statistical forecasting technology, such as, for example, Standard Regression (SR), Support Vector Regression (SVR), Ridge Regression (Ridge), Random Forest (RF), Autoregressive Integrated Moving Average (ARIMA), Vector Auto Regression (VAR), Arbitrage of Forecasting Expert (AFE), Extra-Tree Regression (ETR), Multilayer Perceptron (MLPR), or Vector Error Correction Model (VECM).

In certain embodiments of the biomarker analyzer and cancer (e.g., TNBC) predictor 160, the machine learning platform, including the machine learning model, is trained using a training dataset created based on the various embodiments/examples provided in this disclosure. For instance, a portion of the datasets created based on the various embodiments/examples provided herein can be prepared for training the machine learning model by, for example, removing duplicates, correcting errors, providing any missing values, normalization, data type conversions, data randomizing, addition of annotations, among other things, as will be understood by those skilled in the art. Additionally, the remaining portion of the datasets created based on the various embodiments/examples provided herein can be used to create a validation dataset to validate the machine learning model. In certain embodiments, the original dataset can be split such that 50% of the dataset is used to build the training set and the remaining 50% is used to build the validation dataset. Other ratios are contemplated, such as, for example, but not limited to, 90/10, 80/20, 70/30 or 60/40 for training/validation. The training dataset can be used to train the machine learning model to make predictions correctly consistently. The model can then be validated using the validation dataset. Once trained, the model can be tuned, for example, by hyperparametric tuning, for improved performance.

The biomarker analyzer and cancer (e.g., TNBC) predictor 160 can include a biomarker analysis unit 160A, a TNBC (triple negative breast cancer) prediction unit 160B, and a subject classification unit 160C. Each of the units 160A, 160B, and 160C can include (or be included in) a machine learning platform. In certain embodiments, the biomarker analysis unit 160A is configured to analyze computer-readable data corresponding to a sample from a subject, which may include one or more biomarkers. In certain embodiments, the cancer (e.g., TNBC) prediction unit 160B is configured to predict a prognosis of, and/or a treatment outcome for, the subject based on a result of the analysis of the computer-readable data and generate a risk score. In certain embodiments the subject classification unit 160C is configured to determine a classification of the subject based on the prediction and risk score, including whether the subject is a high risk or low risk for disease progression, relapse, recurrence, and/or death.

In certain embodiments, the biomarker analysis unit 160A is configured to analyze data of a plurality of biomarkers, including, for example, at least two biomarkers, at least three biomarkers, at least four biomarkers, at least five biomarkers, at least six biomarkers, at least seven biomarkers, at least eight biomarkers, at least nine biomarkers, or ten biomarkers selected from the group consisting of ANKRD36, ANKRD36BP2, BSPRY, C12orf65, C2orf49, C1orf198, CCDC114, CLDN4, CUEDC1, ODAD1, CGREF1, DEPDC7, DCLK2, DGKH, DIP2B, DISC1, EMP1, ERH, GADD45B, GLS, GRHL1, GYPC, H2AFX, HRAS, ICAM1, IMPG2, KCNC3, KLF6, KLF7, KRT17, LONRF2, LRBA, LRIT3, LRRC37B, LSM11, LYST, MALAT1, MCM3AP_AS1, MICALL2, MICB, MT2A, MYEF2, N4BP3, NBPF20, NDUFV2, NOTCH2, NOXA1, NPR3, NR6A1, PAK3, PANK3, PARD6B, PEX1, PGBD4, PJA1, PLEKHF1, PNP, PPM1K, PRICKLE1, PRKAB2, PTPRA, PYCARD, RASA1, RASD2, RASGRP1, RHPN2, RILPL2, ROBO1, RORA, SDF4, SERTAD4, SHISA5, SIPA1L2, SLC22A20P, SLC24A3, SLC2A12, SLC39A10, SLC25A40, SLC43A1, SLC45A4, SLC6A20, SPTB, STAG3L3, SUSD3, TAF10, TCTE3, TMC7, TMCC2, TRPS1, TTLL4, TYW5, UBE2 W, WDSUB1, WDYHV1, NTAQ1, ZBED6, ZBTB46, ZC3H13, ZHX2, ZNF217, ZNF233, ZNF248, ZNF469, and ZNF785. The biomarker analysis unit 160A can be configured to analyze RNA and/or protein sequencing data from tumor tissue of cancer (e.g., TNBC) patients.

In certain embodiments, the cancer (e.g., TNBC) prediction unit 160B is configured to determine a score based on the plurality of biomarkers (for example, 6, 7, 8, 9, or 10-gene signatures—(a) DGKH, GADD45B, KLF7, LYST, NR6A1, PYCARD, ROBO1, SLC22A20P, SLC24A3, and SLC45A4; (b) DGKH, DIP2B, EMP1, GADD45B, MT2A, NOTCH2, NR6A1, RORA, SLC22A20P, and SL24A3; (c) GADD45B, LYST, NOXA1, NR6A1, PYCARD, SLC22A20P, SLC24A3, and NTAQ1; (d) DGKH, KLF7, LYST, NR6A1, ROBO1, SLC24A3, and SLC6A20; (e) DGKH, EMP1, GADD45B, LYST, SLC22A20P, SLC24A3, and SLC6A20; (f) CUEDC1, DGKH, EMP1, LYST, NOXA1, SLC22A20P, and SLC6A20; (g) DGKH, KLF7, LYST, NR6A1, PRICKLE1, ROBO1, and SLC24A3; (h) DCLK2, GADD45B, LYST, NR6A1, SLC22A20P, SLC24A3, and NTAQ1; (i) GADD45B, KLF7, LYST, NR6A1, ROBO1, SLC22A20P, and SLC6A20; (j) DCLK2, GADD45B, LYST, NR6A1, SLC22A20P, and NTAQ1; (k) C12orf65, GADD45B, LRBA, LYST, PEX1, PRKAB2, and TYW5; (l) LYST, PEX1, ODAD1, DEPDC7, MICALL2, SLC43A1, and SLC6A20; (m) C12orf65, GADD45B, LYST, PEX1, RASA1, SLC45A4, and NTAQ1; (n) TYW5, DEPDC7, SLC43A1, CGREF1, MICB, HRAS, and MT2A; (o) LRBA, LYST, PEX1, DEPDC7, SLC43A1, MICB, and C2orf49; (p) GADD45B, PEX1, DEPDC7, SLC43A1, LSM11, and PJA1; (q) DEPDC7, SLC43A1, MICB, C2orf49, HRAS, KCNC3, and MT2A; (r) LYST, DEPDC7, SLC43A1, SLC6A20, MICB, and HRAS; (s) PEX1, MICALL2, SLC43A1, RASA1, KCNC3, and LRIT3; (t) GADD45B, SLC43A1, SLC45A4, KCNC3, SHISA5, and SLC25A40; (u) GADD45B, H2AFX, PTPRA, RILPL2, RORA, ZC3H13, and ZHX2; or (v) CLDN4, ERH, GADD45B, GYPC, H2AFX, MT2A, NDUFV2, SDF4, and UBE2 W) based on, for example: RNA and/or protein sequence data; mRNA expression value of 6, 7, 8, 9, or 10-gene signature from RT-PCR; mRNA expression value of 6, 7, 8, 9, or 10-gene signature in fresh frozen tumor tissue; and/or Scoring with mRNA expression value of 6, 7, 8, 9, or 10-gene signature in FFPE tumor tissue.

In a non-limiting embodiment, the machine learning platform in the biomarker analyzer and cancer (e.g., TNBC) predictor 160 was trained based on analysis of gene expression profiles by RNA sequencing using tumor samples from 184 cancer (e.g., TNBC) patients, comprising a training cohort (n=76) and a validation cohort (n=108). Combining weighted gene expression, the biomarker analyzer and cancer (e.g., TNBC) predictor 160 was trained based on the 6, 7, 8, 9, or 10-gene signature ((a) DGKH, GADD45B, KLF7, LYST, NR6A1, PYCARD, ROBO1, SLC22A20P, SLC24A3, and SLC45A4; (b) DGKH, DIP2B, EMP1, GADD45B, MT2A, NOTCH2, NR6A1, RORA, SLC22A20P, and SL24A3; (c) GADD45B, LYST, NOXA1, NR6A1, PYCARD, SLC22A20P, SLC24A3, and NTAQ1; (d) DGKH, KLF7, LYST, NR6A1, ROBO1, SLC24A3, and SLC6A20; (e) DGKH, EMP1, GADD45B, LYST, SLC22A20P, SLC24A3, and SLC6A20; (f) CUEDC1, DGKH, EMP1, LYST, NOXA1, SLC22A20P, and SLC6A20; (g) DGKH, KLF7, LYST, NR6A1, PRICKLE1, ROBO1, and SLC24A3; (h) DCLK2, GADD45B, LYST, NR6A1, SLC22A20P, SLC24A3, and NTAQ1; (i) GADD45B, KLF7, LYST, NR6A1, ROBO1, SLC22A20P, and SLC6A20; (j) DCLK2, GADD45B, LYST, NR6A1, SLC22A20P, and NTAQ1; (k) C12orf65, GADD45B, LRBA, LYST, PEX1, PRKAB2, and TYW5; (l) LYST, PEX1, ODAD1, DEPDC7, MICALL2, SLC43A1, and SLC6A20; (m) C12orf65, GADD45B, LYST, PEX1, RASA1, SLC45A4, and NTAQ1; (n) TYW5, DEPDC7, SLC43A1, CGREF1, MICB, HRAS, and MT2A; (o) LRBA, LYST, PEX1, DEPDC7, SLC43A1, MICB, and C2orf49; (p) GADD45B, PEX1, DEPDC7, SLC43A1, LSM11, and PJA1; (q) DEPDC7, SLC43A1, MICB, C2orf49, HRAS, KCNC3, and MT2A; (r) LYST, DEPDC7, SLC43A1, SLC6A20, MICB, and HRAS; (s) PEX1, MICALL2, SLC43A1, RASA1, KCNC3, and LRIT3; (t) GADD45B, SLC43A1, SLC45A4, KCNC3, SHISA5, and SLC25A40; (u) GADD45B, H2AFX, PTPRA, RILPL2, RORA, ZC3H13, and ZHX2; or (v) CLDN4, ERH, GADD45B, GYPC, H2AFX, MT2A, NDUFV2, SDF4, and UBE2 W), including stratification of the cancer (e.g., TNBC) patients by risk score (for example, sensitivity=90.91%; specificity=100.00%; accuracy=98.68%), which was validated in the validation cohort. The biomarker analyzer and cancer (e.g., TNBC) predictor 160 is configured to identify and/or validate 6, 7, 8, 9, or 10-gene signature set and predict prognosis of patients with early-stage cancer (e.g., TNBC) based on, for example, the transcriptome of primary tumors.

The communication unit 170 can include one or more devices such as, for example, a transmitter 170A, a receiver 170B, a transceiver (not shown), a modulator (not shown), a demodulator (not shown), a modem (not shown), an encoder (not shown), or a decoder (not shown). The communication unit 170 can be configured to communication with one or more communication devices (not shown), such as, for example, a smartphone, a tablet, or a computer. The communication unit 170 can be configured to send cancer (e.g., TNBC) results to the one or more communication devices (not shown) for each patient, including, for example, a risk score, a predicted prognosis of cancer (e.g., TNBC), and subject classification.

The processor 110 can include a computing device, such as, for example, any of various commercially available graphic processing unit devices. Dual microprocessors and other multi-processor architectures can be included in the processor 110. The processor 110 can include a central processing unit (CPU), a graphic processing unit (GPU), a general-purpose GPU (GPGPU), a field programmable gate array (FGPA), an application-specific integrated circuit (ASIC), or a manycore processor.

The processor 110 can be arranged to process instructions for execution within the system 100, including instructions stored in the memory 120. The processor 110 can process instructions to display graphical information for a GUI on an external input/output device, such as a display device coupled to the IO interface 140 or the high-speed interface (not shown). In other implementations, multiple processors and/or multiple buses can be used, as appropriate, along with multiple memories and types of memory.

The system 100 can include a non-transitory computer-readable medium that can hold executable or interpretable computer program code or instructions that, when executed by the processor 110, can cause the steps, processes and methods in this disclosure to be carried out. The computer-readable medium can be contained in the memory 120.

The memory 120 can include a read only memory (ROM) 120A, a random-access memory (RAM) 120B and a hard disk drive (HDD) 120C. A basic input/output system (BIOS) can be stored in the non-volatile memory, which can include, for example, the ROM 120A. The ROM 120A can include an erasable programmable rea-only memory (EPROM) or an electrically erasable programmable read-only memory (EEPROM). The BIOS can contain the basic routines that help to transfer information and instructions between computer assets 105 to 170 in the system 100, such as during start-up.

The RAM 120B can include a dynamic random-access memory (DRAM), a synchronous dynamic random-access memory (SDRAM), a static random-access memory (SRAM), a non-volatile random-access memory (NVRAM), or another high-speed RAM for caching data.

The HDD 120C can include, for example, an enhanced integrated drive electronics (EIDE) drive, a serial advanced technology attachments (SATA) drive, or any suitable hard disk drive for use with big data. The HDD 120C can be configured for external use in a suitable chassis (not shown). The HDD 120C can be connected to the bus 105 by a hard disk drive interface (not shown) and an optical drive interface (not shown), respectively. The hard disk drive interface (not shown) can include a Universal Serial Bus (USB) (not shown), an IEEE 1394 interface (not shown), or any other suitable interface for external applications.

The memory 120 can provide nonvolatile storage of data, data structures, and computer-executable code or instructions. The memory 120 can accommodate the storage of any data in a suitable digital format. The memory 120 can include one or more computer applications that can be used to execute aspects of the architecture described herein. The memory 120 can include, for example, flash memory or NVRAM memory.

One or more computer resources can be contained in the memory 120, including, for example, an operating system (not shown), one or more application programs (not shown), one or more APIs, and program data (not shown). In certain embodiments, the machine learning platform and/or machine learning model can be contained in the memory 120. The APIs can include, for example, JSON APIs, XML APIs, Web APIs, SOAP APIs, RPC APIs, REST APIs, or other utilities or services APIs. Any (or all) of the computer programs can be cached in the RAM 120B as executable sections of computer program code.

The network interface 130 can be connected to a network (not shown). The system 100 can connect to a communicating device (not shown via, for example, the network interface 130 communicating with the communicating device over a communication link. The network interface 130 can be connected to the network via one or more communication links (not shown). The network interface 130 can include a wired or a wireless communication network interface (not shown) or a modem (not shown). When used in a local area network (LAN), the system 100 can be connected to the LAN network through the wired or wireless communication network interface; and, when used in a wide area network (WAN), the system 100 can be connected to the WAN network through the modem. The network can include a LAN, a WAN, the Internet, or any other network. The modem (not shown) can be internal or external and wired or wireless. The modem can be connected to the bus 105 via, for example, a serial port interface (not shown).

The IO interface 140 can be arranged to receive commands and data from a user. The IO interface 140 can be arranged to connect to or communication with one or more input/output devices (not shown), including, for example, a keyboard (not shown), a mouse (not shown), a pointer (not shown), a microphone (not shown), a speaker (not shown), or a display (not shown). The received commands and data can be forwarded from the IO interface 140 as instruction and data signals via the bus 105 to any computer asset in the system 100.

The driver suite 150 can include an audio driver 150A and a video driver 150B. The audio driver 150A can include a sound card, a sound driver (not shown), an IVR unit, or any other device necessary to render a sound signal on a sound production device (not shown), such as for example, a speaker (not shown). The video driver 150B can include a video card (not shown), a graphics driver (not shown), a video adaptor (not shown), or any other device necessary to render an image signal on a display device (not shown).

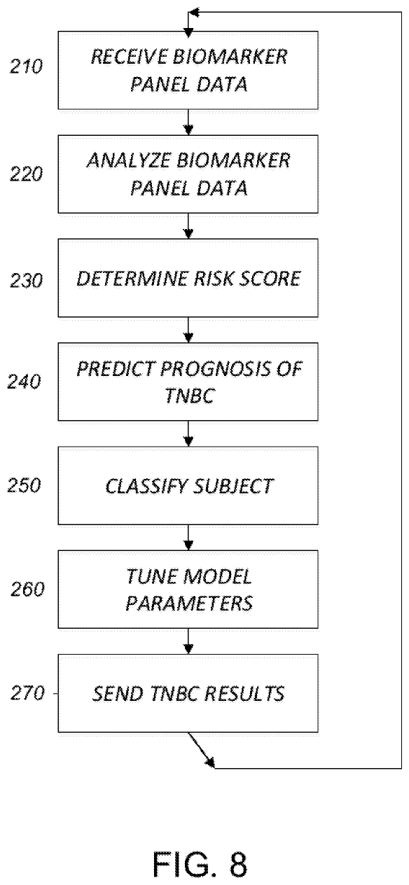

FIG. 8 depicts a non-limiting embodiment of a computer-implemented process that can be performed by the system 100. Referring to FIGS. 7 and 8 together, the system 100 can receive data of a panel of biomarkers for a subject/patient (Step 210). The system 100 can receive the biomarker panel data via the network interface 130, IO interface 140 or receiver (RX) 170B. The received data can be input to the biomarker analyzer and cancer (e.g., TNBC) predictor 160, which can analyze the data by the biomarker analysis unit 160A (Step 220). The biomarker panel data can be analyzed for patterns, including, for example, the presence of at least two, at least three, at least four, at least five, at least six, at least seven, at least eight, at least nine, or ten of a 6, 7, 8, 9, or 10-gene signature-namely, (a) DGKH, GADD45B, KLF7, LYST, NR6A1, PYCARD, ROBO1, SLC22A20P, SLC24A3, and SLC45A4; (b) DGKH, DIP2B, EMP1, GADD45B, MT2A, NOTCH2, NR6A1, RORA, SLC22A20P, and SL24A3; (c) GADD45B, LYST, NOXA1, NR6A1, PYCARD, SLC22A20P, SLC24A3, and NTAQ1; (d) DGKH, KLF7, LYST, NR6A1, ROBO1, SLC24A3, and SLC6A20; (e) DGKH, EMP1, GADD45B, LYST, SLC22A20P, SLC24A3, and SLC6A20; (f) CUEDC1, DGKH, EMP1, LYST, NOXA1, SLC22A20P, and SLC6A20; (g) DGKH, KLF7, LYST, NR6A1, PRICKLE1, ROBO1, and SLC24A3; (h) DCLK2, GADD45B, LYST, NR6A1, SLC22A20P, SLC24A3, and NTAQ1; (i) GADD45B, KLF7, LYST, NR6A1, ROBO1, SLC22A20P, and SLC6A20; (j) DCLK2, GADD45B, LYST, NR6A1, SLC22A20P, and NTAQ1; (k) C12orf65, GADD45B, LRBA, LYST, PEX1, PRKAB2, and TYW5; (l) LYST, PEX1, ODAD1, DEPDC7, MICALL2, SLC43A1, and SLC6A20; (m) C12orf65, GADD45B, LYST, PEX1, RASA1, SLC45A4, and NTAQ1; (n) TYW5, DEPDC7, SLC43A1, CGREF1, MICB, HRAS, and MT2A; (o) LRBA, LYST, PEX1, DEPDC7, SLC43 A1, MICB, and C2orf49; (p) GADD45B, PEX1, DEPDC7, SLC43A1, LSM11, and PJA1; (q) DEPDC7, SLC43A1, MICB, C2orf49, HRAS, KCNC3, and MT2A; (r) LYST, DEPDC7, SLC43A1, SLC6A20, MICB, and HRAS; (s) PEX1, MICALL2, SLC43A1, RASA1, KCNC3, and LRIT3; (t) GADD45B, SLC43A1, SLC45A4, KCNC3, SHISA5, and SLC25A40; (u) GADD45B, H2AFX, PTPRA, RILPL2, RORA, ZC3H13, and ZHX2; or (v) CLDN4, ERH, GADD45B, GYPC, H2AFX, MT2A, NDUFV2, SDF4, and UBE2 W.

Based on the analysis, the cancer (e.g., TNBC) prediction unit 160B can determine a risk score (Step 230) and predict a prognosis of cancer (e.g., TNBC) for the particular subject/patient (Step 240). Based on the risk score and prediction, the subject classification unit 160C can classify the subject/patient as high or low risk (Step 250). In certain embodiments, the classification can have other values, other than high and low risks, such as, for example, a numerical value that represents the predicted likelihood of a cancer (e.g., TNBC) prognosis for the subject/patient. The cancer (e.g., TNBC) prediction results can be packaged and sent to one or more communication devices (not shown), such as, for example, a smartphone, a tablet, or a computer (Step 270), where the cancer (e.g., TNBC) prediction results can be rendered, for example, on a display, and/or used to create a treatment regimen for the particular subject/patient.

In certain embodiments, the biomarker analyzer and cancer (e.g., TNBC) predictor 160 can be configured to provide a treatment regimen based on the cancer (e.g., TNBC) results.

In certain embodiments, the prediction, risk score and/or classification determined by the machine learning model in biomarker analyzer and cancer (e.g., TNBC) predictor 160 can be used to tune the parametric values of the model (Step 260).

The term “backbone,” as used in this disclosure, means a transmission medium or infrastructure that interconnects one or more computing devices or communicating devices to provide a path that conveys data packets or instructions between the computing devices or communicating devices. The backbone can include a network. The backbone can include an ethernet TCP/IP. The backbone can include a distributed backbone, a collapsed backbone, a parallel backbone or a serial backbone.

The term “bus,” as used in this disclosure, means any of several types of bus structures that can further interconnect to a memory bus (with or without a memory controller), a peripheral bus, or a local bus using any of a variety of commercially available bus architectures. The term “bus” can include a backbone.

The term “communicating device” or “communication device,” as used in this disclosure, means any computing device, hardware, or computing resource that can transmit or receive digital or analog signals or data packets, or instruction signals or data signals over a communication link. The device can be portable or stationary.

The term “communication link,” as used in this disclosure, means a wired and/or wireless medium that conveys data or information between at least two points. The wired or wireless medium can include, for example, a metallic conductor link, a radio frequency (RF) communication link, an Infrared (IR) communication link, or an optical communication link. The RF communication link can include, for example, GSM voice calls, SMS, EMS, MMS messaging, CDMA, TDMA, PDC, WCDMA, CDMA2000, GPRS, WiFi, WiMAX, IEEE 802.11, DECT, 0G, 1G, 2G, 3G, 4G or 5G cellular standards, or Bluetooth. A communication link can include, for example, an RS-232, RS-422, RS-485, or any other suitable interface.

The terms “computer” or “computing device,” as used in this disclosure, means any machine, device, circuit, component, or module, or any system of machines, devices, circuits, components, or modules, which can be capable of manipulating data according to one or more instructions, such as, for example, without limitation, a processor, an application specific integrated circuit (ASIC), a field programmable gate array (FPGA), a microprocessor (μP), a central processing unit (CPU), a graphic processing unit (GPU), a general purpose computer, a super computer, a personal computer, a laptop computer, a palmtop computer, a notebook computer, a smart phone, a mobile phone, a tablet, a desktop computer, a workstation computer, a server, a server farm, a computer cloud, or an array of processors, ASICS, FPGAs, μPs, CPUs, GPUs, general purpose computers, super computers, personal computers, laptop computers, palmtop computers, notebook computers, desktop computers, workstation computers, or servers. A computer or computing device can include hardware, firmware, or software that can transmit or receive data packets or instructions over a communication link. The computer or computing device can be portable or stationary.

The term “computer asset,” as used in this disclosure, means a computer resource, a computing device, a communicating device, or a computer-readable medium.

The term “computer resource,” as used in this disclosure, means software, a software application, a web application, a webpage, a document, a file, a record, an application program(ming) interface (API), web content, a computer application, a computer program, computer code, machine executable instructions, or firmware. A computer resource can include an information resource. A computer resource can include machine instructions for a programmable computing device and can be implemented in a high-level procedural or object-oriented programming language, or in assembly/machine language.

The term “computer-readable medium,” as used in this disclosure, means any storage medium that participates in providing data (for example, instructions) that can be read by a computer. Such a medium can take many forms, including non-volatile media and volatile media. Non-volatile media can include, for example, optical or magnetic disks and other persistent memory. Volatile media can include dynamic random-access memory (DRAM). Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read. The computer-readable medium can include a “Cloud,” which includes a distribution of files across multiple (e.g., thousands of) memory caches on multiple (e.g., thousands of) computers. The computer-readable medium can include magnetic discs, optical disks, memory, or Programmable Logic Devices (PLDs).

Various forms of computer readable media can be involved in carrying sequences of instructions to a computer. For example, sequences of instruction (i) can be delivered from a RAM to a processor, (ii) can be carried over a wireless transmission medium, and/or (iii) can be formatted according to numerous formats, standards or protocols, including, for example, WiFi, WiMAX, IEEE 802.11, DECT, 0G, 1G, 2G, 3G, 4G, or 5G cellular standards, or Bluetooth.

The term “database,” as used in this disclosure, means any combination of software and/or hardware, including at least one application and/or at least one computer. The database can include a structured collection of records or data organized according to a database model, such as, for example, but not limited to at least one of a relational model, a hierarchical model, or a network model. The database can include a database management system application (DBMS). The at least one application may include, but is not limited to, for example, an application program that can accept connections to service requests from clients by sending back responses to the clients. The database can be configured to run the at least one application, often under heavy workloads, unattended, for extended periods of time with minimal human direction.

The term “network,” as used in this disclosure means, but is not limited to, for example, at least one of a personal area network (PAN), a local area network (LAN), a wireless local area network (WLAN), a campus area network (CAN), a metropolitan area network (MAN), a wide area network (WAN), a global area network (GAN), a broadband area network (BAN), a cellular network, a storage-area network (SAN), a system-area network, a passive optical local area network (POLAN), an enterprise private network (EPN), a virtual private network (VPN), the Internet, or any combination of the foregoing, any of which can be configured to communicate data via a wireless and/or a wired communication medium. These networks can run a variety of protocols, including, but not limited to, for example, Ethernet, IP, IPX, TCP, UDP, SPX, IP, IRC, HTTP, FTP, Telnet, SMTP, DNS, ARP, ICMP.

The term “server,” as used in this disclosure, means any combination of software and/or hardware, including at least one application and/or at least one computer to perform services for connected clients as part of a client-server architecture. The at least one server application can include, but is not limited to, for example, an application program that can accept connections to service requests from clients by sending back responses to the clients. The server can be configured to run the at least one application, often under heavy workloads, unattended, for extended periods of time with minimal human direction. The server can include a plurality of computers configured, with the at least one application being divided among the computers depending upon the workload. For example, under light loading, the at least one application can run on a single computer. However, under heavy loading, multiple computers can be required to run the at least one application. The server, or any if its computers, can also be used as a workstation.

Devices that are in communication with each other need not be in continuous communication with each other, unless expressly specified otherwise. In addition, devices that are in communication with each other may communicate directly or indirectly through one or more intermediaries.

Although process steps, method steps, algorithms, or the like, may be described in a sequential or a parallel order, such processes, methods and algorithms may be configured to work in alternate orders. In other words, any sequence or order of steps that may be described in a sequential order does not necessarily indicate a requirement that the steps be performed in that order; some steps may be performed simultaneously. Similarly, if a sequence or order of steps is described in a parallel (or simultaneous) order, such steps can be performed in a sequential order. The steps of the processes, methods or algorithms described herein may be performed in any order practical.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

### REFERENCES

1 Collaboration, G. B. o. D. C. Global, Regional, and National Cancer Incidence, Mortality, Years of Life Lost, Years Lived With Disability, and Disability-Adjusted Life-Years for 29 Cancer Groups, 1990 to 2016: A Systematic Analysis for the Global Burden of Disease Study. JAMA Oncology 4, 1553-1568, doi: 10.1001/jamaoncol.2018.2706 (2018).
2 Sung, H. et al. Global Cancer Statistics 2020: GLOBOCAN Estimates of Incidence and Mortality Worldwide for 36 Cancers in 185 Countries. CA: a cancer journal for clinicians 71, 209-249, doi: https://doi.org/10.3322/caac.21660 (2021).
3 Hong, S. et al. Cancer Statistics in Korea: Incidence, Mortality, Survival, and Prevalence in 2017. Cancer Res Treat 52, 335-350, doi: 10.4143/crt.2020.206 (2020).
4 Allemani, C. et al. Global surveillance of trends in cancer survival 2000-14 (CONCORD-3): analysis of individual records for 37 513 025 patients diagnosed with one of 18 cancers from 322 population-based registries in 71 countries. Lancet (London, England) 391, 1023-1075, doi: 10.1016/s0140-6736 (17) 33326-3 (2018).
5 Sparano, J. A. et al. Adjuvant Chemotherapy Guided by a 21-Gene Expression Assay in Breast Cancer. The New England journal of medicine 379, 111-121, doi: 10.1056/NEJMoa1804710 (2018).
6 Henry, N. L. et al. Role of Patient and Disease Factors in Adjuvant Systemic Therapy Decision Making for Early-Stage, Operable Breast Cancer: Update of the ASCO Endorsement of the Cancer Care Ontario Guideline. Journal of clinical oncology: official journal of the American Society of Clinical Oncology 37, 1965-1977, doi: 10.1200/jco.19.00948 (2019).
7 Carey, L. A. et al. The triple negative paradox: primary tumor chemosensitivity of breast cancer subtypes. Clinical cancer research: an official journal of the American Association for Cancer Research 13, 2329-2334, doi: 10.1158/1078-0432.Ccr-06-1109 (2007).
8 Perou, C. M. et al. Molecular portraits of human breast tumours. Nature 406, 747-752, doi: 10.1038/35021093 (2000).
9 Park, J. et al. Nonphosphorylatable PEA15 mutant inhibits epithelial-mesenchymal transition in triple-negative breast cancer partly through the regulation of IL-8 expression. Breast cancer research and treatment, doi: 10.1007/s10549-021-06316-2 (2021).
10 Prat, A. et al. Phenotypic and molecular characterization of the claudin-low intrinsic subtype of breast cancer. Breast cancer research: BCR 12, R68, doi: 10.1186/bcr2635 (2010).
11 Tutt, A. et al. Carboplatin in BRCA1/2-mutated and triple-negative breast cancer BRCAness subgroups: the TNT Trial. Nature Medicine 24, 628-637, doi: 10.1038/s41591-018-0009-7 (2018).
12 Wolff, A. C. et al. Recommendations for Human Epidermal Growth Factor Receptor 2 Testing in Breast Cancer: American Society of Clinical Oncology/College of American Pathologists Clinical Practice Guideline Update. Journal of Clinical Oncology 31, 3997-4013, doi: 10.1200/jco.2013.50.9984 (2013).
13 Allison, K. H. et al. Estrogen and Progesterone Receptor Testing in Breast Cancer: American Society of Clinical Oncology/College of American Pathologists Guideline Update. Archives of Pathology & Laboratory Medicine 144, 545-563, doi: 10.5858/arpa.2019-0904-SA (2020).
14 Park, I. J., Yu, Y. S. & Mustafa, B. A Nine-Gene Signature for Predicting the Response to Preoperative Chemoradiotherapy in Patients with Locally Advanced Rectal Cancer. 12, doi: 10.3390/cancers12040800 (2020).
15 Picornell, A. C. et al. Breast cancer PAM50 signature: correlation and concordance between RNA-Seq and digital multiplexed gene expression technologies in a triple negative breast cancer series. BMC genomics 20, 452, doi: 10.1186/s12864-019-5849-0 (2019).
16 Raj-Kumar, P. K. et al. PCA-PAM50 improves consistency between breast cancer intrinsic and clinical subtyping reclassifying a subset of luminal A tumors as luminal B. Scientific reports 9, 7956, doi: 10.1038/s41598-019-44339-4 (2019).
17 Bolotin, D. A. et al. Antigen receptor repertoire profiling from RNA-seq data. 35, 908-911, doi: 10.1038/nbt.3979 (2017).
18 Han, J. et al. TCR Repertoire Diversity of Peripheral PD-1(+) CD8(+) T Cells Predicts Clinical Outcomes after Immunotherapy in Patients with Non-Small Cell Lung Cancer. Cancer immunology research 8, 146-154, doi: 10.1158/2326-6066.cir-19-0398 (2020).
19 Lerebours, F. et al. NF-kappa B genes have a major role in inflammatory breast cancer. BMC cancer 8, 41, doi: 10.1186/1471-2407-8-41 (2008).
20 Bjarnadottir, O. et al. Global Transcriptional Changes Following Statin Treatment in Breast Cancer. Clinical cancer research: an official journal of the American Association for Cancer Research 21, 3402-3411, doi: 10.1158/1078-0432.ccr-14-1403 (2015).
21 Ouhtit, A. et al. Simultaneous inhibition of cell-cycle, proliferation, survival, metastatic pathways and induction of apoptosis in breast cancer cells by a phytochemical super-cocktail: genes that underpin its mode of action. Journal of Cancer 4, 703-715, doi: 10.7150/jca.7235 (2013).
22 Wang, J.-W. et al. Deregulated expression of LRBA facilitates cancer cell growth. Oncogene 23, 4089-4097, doi: 10.1038/sj.onc.1207567 (2004).
23 Tozlu, S. et al. Identification of novel genes that co-cluster with estrogen receptor alpha in breast tumor biopsy specimens, using a large-scale real-time reverse transcription-PCR approach. Endocrine-related cancer 13, 1109-1120, doi: 10.1677/erc.1.01120 (2006).
24 Andres, S. A., Brock, G. N. & Wittliff, J. L. Interrogating differences in expression of targeted gene sets to predict breast cancer outcome. BMC cancer 13, 326, doi: 10.1186/1471-2407-13-326 (2013).
25 Wang, J. W. et al. Deregulated expression of LRBA facilitates cancer cell growth. Oncogene 23, 4089-4097, doi: 10.1038/sj.onc.1207567 (2004).
26 Andres, S. A., Smolenkova, I. A. & Wittliff, J. L. Gender-associated expression of tumor markers and a small gene set in breast carcinoma. Breast 23, 226-233, doi: 10.1016/j.breast.2014.02.007 (2014).
27 Holt, O. J., Gallo, F. & Griffiths, G. M. Regulating Secretory Lysosomes. The Journal of Biochemistry 140, 7-12, doi: 10.1093/jb/mvj126 (2006).
28 Bong, I. P., Ng, C. C., Fakiruddin, S. K., Lim, M. N. & Zakaria, Z. Small interfering RNA-mediated silencing of nicotinamide phosphoribosyltransferase (NAMPT) and lysosomal trafficking regulator (LYST) induce growth inhibition and apoptosis in human multiple myeloma cells: A preliminary study. Bosnian journal of basic medical sciences 16, 268-275, doi: 10.17305/bjbms.2016.1568 (2016).
29 Sara, M. PEX1 and PEX7 Genes are Necessary for Completion of Immune Pathways and Survival Post-Infection. STEM Fellowship Journal 6, 5-11, doi: 10.17975/sfj-2020-002 (2020).
30 Pang, E. Y. et al. Identification of PFTAIRE protein kinase 1, a novel cell division cycle-2 related gene, in the motile phenotype of hepatocellular carcinoma cells. Hepatology 46, 436-445, doi: 10.1002/hep.21691 (2007).
31 Chen, Y. et al. Identification of druggable cancer driver genes amplified across TCGA datasets. PLoS One 9, e98293, doi: 10.1371/journal.pone. 0098293 (2014).
32 Del Rizzo, P. A., Krishnan, S. & Trievel, R. C. Crystal structure and functional analysis of JMJD5 indicate an alternate specificity and function. Molecular and cellular biology 32, 4044-4052, doi: 10.1128/MCB.00513-12 (2012).
33 Oh, S., Shin, S. & Janknecht, R. The small members of the JMJD protein family: Enzymatic jewels or jinxes? Biochimica et Biophysica Acta (BBA)—Reviews on Cancer 1871, 406-418, doi: https://doi.org/10.1016/j.bbcan.2019.04.002 (2019).
34 Tucci, A. et al. Novel C12orf65 mutations in patients with axonal neuropathy and optic atrophy. Journal of neurology, neurosurgery, and psychiatry 85, 486-492, doi: 10.1136/jnnp-2013-306387 (2014).
35 Pai, T. et al. Evidence for the association of Epstein-Barr Virus in breast cancer in Indian patients using in-situ hybridization technique. The breast journal 24, 16-22, doi: 10.1111/tbj.12828 (2018).
36 Horakova, D. et al. Risks and protective factors for triple negative breast cancer with a focus on micronutrients and infections. Biomedical papers of the Medical Faculty of the University Palacky, Olomouc, Czechoslovakia 162, 83-89, doi: 10.5507/bp.2018.014 (2018).
37 Chen, X. et al. Cyclin E Overexpression Sensitizes Triple-Negative Breast Cancer to Weel Kinase Inhibition. Clinical cancer research: an official journal of the American Association for Cancer Research 24, 6594-6610, doi: 10.1158/1078-0432.CCR-18-1446 (2018).
38 Sharma, P. Biology and Management of Patients With Triple-Negative Breast Cancer. The oncologist 21, 1050-1062, doi: 10.1634/theoncologist.2016-0067 (2016).
39 Masuda, N. et al. Adjuvant Capecitabine for Breast Cancer after Preoperative Chemotherapy. The New England journal of medicine 376, 2147-2159, doi: 10.1056/NEJMoa1612645 (2017).

1. An isolated set of probes capable of detecting a panel of biomarkers comprising at least two biomarkers selected from the group consisting of ankyrin repeat domain 36 (ANKRD36), ankyrin repeat domain 36B pseudogene 2 (ANKRD36BP2), B-box and SPRY domain-containing (BSPRY), chromosome 12 open reading frame 65 (C12orf65), chromosome 2 open reading frame 49 (C2orf49), chromosome 1 open reading frame 198 (Clorf198), coiled-coil domain containing 114 (CCDC114), claudin 4 (CLDN4), CUE domain containing 1 (CUEDC1), outer dynein arm docking complex subunit 1 (ODAD1), cell growth regulator with EF-hand domain 1 (CGREF1), DEP domain containing 7 (DEPDC7), doublecortin like kinase 2 (DCLK2), diacylglycerol kinase (DGKH), disco interacting protein 2 homolog B (DIP2B), disrupted-in-schizophrenia 1 (DISC1), epithelial membrane protein 1 (EMP1), ERH mRNA splicing and mitosis factor (ERH), growth arrest and DNA damage inducible beta (GADD45B), glutaminase (GLS), grainyhead like transcription factor 1 (GRHL1), glycophorin C (GYPC), H2A histone family member X (H2AFX), HRas proto-oncogene (HRAS), intracellular adhesion molecule 1 (ICAM1), interphotoreceptor matrix proteoglycan 2 (IMPG2), potassium voltage-gated channel subfamily C member 3 (KCNC3), kruppel like factor 6 (KLF6), kruppel like factor 7 (KLF7), keratin 17 (KRT17), LON peptidase N-terminal domain and RING finger protein 2 (LONRF2), LPS responsive beige-like anchor protein (LRBA), leucine rich repeat, Ig-like and transmembrane domains 3 (LRIT3), leucine rich repeat containing 37B (LRRC37B), LSM11, U7 small nuclear RNA associated (LSM11), lysosomal trafficking regulator (LYST), metastasis associated lung adenocarcinoma transcript 1 (MALAT1), minichromosome maintenance complex component 3 associated protein antisense RNA 1 (MCM3AP_AS1), MICAL like 2 (MICALL2), MHC class I polypeptide-related sequence B (MICB), metallothionein 2A (MT2A), myelin expression factor 2 (MYEF2), NEDD4 binding protein 3 (N4BP3), neuroblastoma breakpoint family member 20 (NBPF20), NADH: Ubiquinone Oxidoreductase Core Subunit V2 (NDUFV2), neurogenic locus notch homolog protein 2 (NOTCH2), NADPH oxidase activator 1 (NOXA1), natriuretic peptide receptor 3 (NPR3), nuclear receptor subfamily 6 group A member 1 (NR6A1), P21 (RAC1) activated kinase 3 (PAK3), pantothenate kinase 3 (PANK3), par-6 family cell polarity regulator beta (PARD6B), peroxisomal biogenesis factor 1 (PEX1), piggyBac transposable element derived 4 (PGBD4), praja ring finger ubiquitin ligase 1 (PJA1), pleckstrin homology and FYVE domain containing 1 (PLEKHF1), purine nucleoside phosphorylase (PNP), protein tyrosine phosphatase receptor type A (PTPRA), protein phosphatase, Mg2+/Mn2+ dependent 1K (PPM1K), prickle planar cell polarity protein 1 (PRICKLE1), protein kinase AMP-activated non-catalytic subunit beta 2 (PRKAB2), PYD and CARD domain containing (PYCARD), RAS p21 protein activator 1 (RASA1), RASD family member 2 (RASD2), RAS guanyl releasing protein 1 (RASGRP1), rhophilin RHO GTPase binding protein 2 (RHPN2), Rab interacting lysosomal protein like 2 (RILPL2), roundabout guidance receptor 1 (ROBO1), RAR related orphan receptor A (RORA), Stromal cell derived factor 4 (SDF4), SERTA domain containing 4 (SERTAD4), shisa family member 5 (SHISA5), signal induced proliferation associated 1 like 2 (SIPA1L2), solute carrier family 22 member 20 (SLC22A20P), solute carrier family 24 member 3 (SLC24A3), solute carrier family 2 member 12 (SLC2A12), solute carrier family 39 member 10 (SLC39A10), solute carrier family 25 member 40 (SLC25A40), solute carrier family 43 member 1 (SLC43A1), solute carrier family 45 member 4 (SLC45A4), solute carrier family 6 member 20 (SLC6A20), spectrin beta, erthrocytic (SPTB), stromal antigen 3-like 3 (STAG3L3), sushi domain containing 3 (SUSD3), TATA-Box binding protein associated factor 10 (TAF10), t-complex-associated testis expressed 3 (TCTE3), transmembrane channel like 7 (TMC7), transmembrane and coiled-coil domain family 2 (TMCC2), transcriptional repressor GATA binding 1 (TRPS1), tubulin tyrosine ligase like 4 (TTLL4), tRNA-yW synthesizing protein 5 (TYW5), ubiquitin conjugating enzyme E2 W (UBE2 W), WD repeat, sterile alpha motif and U-box domain containing 1 (WDSUB1), N-protein N-terminal glutamine amidohydrolase (WDYHV1), N-terminal glutamine amidase 1 (NTAQ1), zinc finger BED-type containing 6 (ZBED6), zinc finger and BTB domain containing 46 (ZBTB46), zinc finger CCCH-type containing 13 (ZC3H13), zinc fingers and homeoboxes 2 (ZHX2), zinc finger protein 217 (ZNF217), zinc finger protein 233 (ZNF233), zinc finger protein 248 (ZNF248), zinc finger protein 469 (ZNF469), and zinc finger protein 785 (ZNF785).

2. The isolated set of probes of claim 1, wherein the panel of biomarkers comprises at least two biomarkers selected from the group consisting of DGKH, KLF7, NR6A1, PYCARD, ROBO1, SLC22A20P, SLC24A3, DIP2B, EMP1, NOTCH2, RORA, NOXA1, CUEDC1, PRICKLE1, DCKL2, C12orf65, GADD45B, LRBA, LYST, PEX1, PRKAB2, TYW5, ODAD1, DEPDC7, MICALL2, SLC43A1, SLC6A20, RASA1, SLC45A4, NTAQ1, CGREF1, MICB, LSM11, PJA1, C2orf49, HRAS, KCNC3, MT2A, LRIT3, SHISA5, SLC25A40, H2AFX, PTPRA, RILPL2, ZC3CH13, ZHX2, CDLN4, ERH, GYPC, MTA2, NDUFV2, SDF4, and UBE2 W.

3. The isolated set of probes of claim 1, wherein the panel of biomarkers comprises at least three biomarkers, at least four biomarkers, at least five biomarkers, at least six biomarkers, at least seven biomarkers, at least eight biomarkers, at least nine biomarkers, or ten biomarkers selected from a biomarker signature as follows:
a) DGKH, GADD45B, KLF7, LYST, NR6A1, PYCARD, ROBO1, SLC22A20P, SLC24A3, and SLC45A4;
b) DGKH, DIP2B, EMP1, GADD45B, MT2A, NOTCH2, NR6A1, RORA, SLC22A20P, and SL24A3;
c) GADD45B, LYST, NOXA1, NR6A1, PYCARD, SLC22A20P, SLC24A3, and NTAQ1;
d) DGKH, KLF7, LYST, NR6A1, ROBO1, SLC24A3, and SLC6A20;
e) DGKH, EMP1, GADD45B, LYST, SLC22A20P, SLC24A3, and SLC6A20;
f) CUEDC1, DGKH, EMP1, LYST, NOXA1, SLC22A20P, and SLC6A20;
g) DGKH, KLF7, LYST, NR6A1, PRICKLE1, ROBO1, and SLC24A3;
h) DCLK2, GADD45B, LYST, NR6A1, SLC22A20P, SLC24A3, and NTAQ1;
i) GADD45B, KLF7, LYST, NR6A1, ROBO1, SLC22A20P, and SLC6A20;
j) DCLK2, GADD45B, LYST, NR6A1, SLC22A20P, and NTAQ1;
k) C12orf65, GADD45B, LRBA, LYST, PEX1, PRKAB2, and TYW5;
l) LYST, PEX1, ODAD1, DEPDC7, MICALL2, SLC43A1, and SLC6A20;
m) C12orf65, GADD45B, LYST, PEX1, RASA1, SLC45A4, and NTAQ1;
n) TYW5, DEPDC7, SLC43A1, CGREF1, MICB, HRAS, and MT2A;
o) LRBA, LYST, PEX1, DEPDC7, SLC43A1, MICB, and C2orf49;
p) GADD45B, PEX1, DEPDC7, SLC43A1, LSM11, and PJA1;
q) DEPDC7, SLC43A1, MICB, C2orf49, HRAS, KCNC3, and MT2A;
r) LYST, DEPDC7, SLC43A1, SLC6A20, MICB, and HRAS;
s) PEX1, MICALL2, SLC43A1, RASA1, KCNC3, and LRIT3;
t) GADD45B, SLC43A1, SLC45A4, KCNC3, SHISA5, and SLC25A40;
u) GADD45B, H2AF, PTPRA, RILPL2, RORA, ZC3H13, and ZHX2; or
v) CLDN4, ERH, GADD45B, GYPC, H2AFX, MT2A, NDUFV2, SDF4, and UBE2 W.

4. The isolated set of probes of claim 1, wherein the probe is selected from the group consisting of an aptamer, an antibody, an affibody, a peptide, a protein, an organic molecule, and a nucleic acid.

5. A method for predicting a disease prognosis and/or a treatment outcome for a subject diagnosed with cancer, the method comprising:
a) obtaining a sample from the subject;
b) contacting the sample with the isolated set of probes of claim 1 to detect a panel of biomarkers in the sample;
c) analyzing a pattern of the panel of biomarkers to determine a risk score for the subject.

6. The method of claim 5, wherein the method further comprises:
d) classifying the subject as high risk or low risk based on the risk score.

7. The method of claim 5, wherein the cancer is breast cancer.

8. The method of claim 7, wherein the breast cancer is triple-negative breast cancer (TNBC).

9. The method of claim 8, wherein the TNBC is early-stage TNBC.

10. The method of claim 8, further comprising treating the TNBC in the subject based on the classification of the subject.

11. The method of claim 5, wherein the subject is high risk and the method further comprises an advanced, strengthened, or standard form of treatment for TNBC comprising surgery and/or administering chemotherapeutic agents, radiotherapy, immunotherapeutic agents, any novel therapeutics, or a combination of treatments.

12. The method of claim 11, wherein the immunotherapeutic agent is an immune checkpoint inhibitor selected from pembrolizumab, atezolizumab, nivolumab, ipilimumab, durvalumab, and/or avelumab.

13. (canceled)

14. The method of claim 11, wherein the chemotherapeutic agents comprise capecitabine, doxorubicin, cyclophosphamide, docetaxel, olaparib, carboplatin, paclitaxel, epirubicin, methotrexate, and/or fluorouracil.

15. The method of claim 5, wherein the subject is low risk and the method further comprises administering the standard or attenuated treatment for TNBC comprising surgery only or surgery and/or administering chemotherapeutic agents, radiotherapy, immunotherapy, any novel treatment, or a combination of treatments.

16. The method of claim 15, wherein the immunotherapeutic agent is an immune checkpoint inhibitor selected from pembrolizumab, atezolizumab, nivolumab, ipilimumab, durvalumab, and/or avelumab.

17. (canceled)

18. The method of claim 15, wherein the chemotherapeutic agents comprise capecitabine, doxorubicin, cyclophosphamide, docetaxel, olaparib, carboplatin, paclitaxel, epirubicin, methotrexate, and/or fluorouracil.

19. The method of claim 5, wherein the sample is a tissue sample, a blood sample, or a urine sample.

20. (canceled)

21. A kit for predicting a disease prognosis and/or a treatment outcome for a subject diagnosed with triple negative breast cancer (TNBC), the kit comprising:
a) an isolated set of probes capable of detecting a panel of biomarkers comprising at least two biomarkers selected from the group consisting of ankyrin repeat domain 36 (ANKRD36), ankyrin repeat domain 36B pseudogene 2 (ANKRD36BP2), B-box and SPRY domain-containing (BSPRY), chromosome 12 open reading frame 65 (C12orf65), chromosome 2 open reading frame 49 (C2orf49), chromosome 1 open reading frame 198 (Clorf198), coiled-coil domain containing 114 (CCDC114), claudin 4 (CLDN4), CUE domain containing 1 (CUEDC1), outer dynein arm docking complex subunit 1 (ODAD1), cell growth regulator with EF-hand domain 1 (CGREF1), DEP domain containing 7 (DEPDC7), doublecortin like kinase 2 (DCLK2), diacylglycerol kinase (DGKH), disco interacting protein 2 homolog B (DIP2B), disrupted-in-schizophrenia 1 (DISC1), epithelial membrane protein 1 (EMP1), ERH mRNA splicing and mitosis factor (ERH), growth arrest and DNA damage inducible beta (GADD45B), glutaminase (GLS), grainyhead like transcription factor 1 (GRHL1), glycophorin C (GYPC), H2A histone family member X (H2AFX), HRas proto-oncogene (HRAS), intracellular adhesion molecule 1 (ICAM1), interphotoreceptor matrix proteoglycan 2 (IMPG2), potassium voltage-gated channel subfamily C member 3 (KCNC3), kruppel like factor 6 (KLF6), kruppel like factor 7 (KLF7), keratin 17 (KRT17), LON peptidase N-terminal domain and RING finger protein 2 (LONRF2), LPS responsive beige-like anchor protein (LRBA), leucine rich repeat, Ig-like and transmembrane domains 3 (LRIT3), leucine rich repeat containing 37B (LRRC37B), LSM11, U7 small nuclear RNA associated (LSM11), lysosomal trafficking regulator (LYST), metastasis associated lung adenocarcinoma transcript 1 (MALAT1), minichromosome maintenance complex component 3 associated protein antisense RNA 1 (MCM3AP_AS1), MICAL like 2 (MICALL2), MHC class I polypeptide-related sequence B (MICB), metallothionein 2A (MT2A), myelin expression factor 2 (MYEF2), NEDD4 binding protein 3 (N4BP3), neuroblastoma breakpoint family member 20 (NBPF20), NADH: Ubiquinone Oxidoreductase Core Subunit V2 (NDUFV2), neurogenic locus notch homolog protein 2 (NOTCH2), NADPH oxidase activator 1 (NOXA1), natriuretic peptide receptor 3 (NPR3), nuclear receptor subfamily 6 group A member 1 (NR6A1), P21 (RAC1) activated kinase 3 (PAK3), pantothenate kinase 3 (PANK3), par-6 family cell polarity regulator beta (PARD6B), peroxisomal biogenesis factor 1 (PEX1), piggyBac transposable element derived 4 (PGBD4), praja ring finger ubiquitin ligase 1 (PJA1), pleckstrin homology and FYVE domain containing 1 (PLEKHF1), purine nucleoside phosphorylase (PNP), protein tyrosine phosphatase receptor type A (PTPRA), protein phosphatase, Mg2+/Mn2+ dependent 1K (PPM1K), prickle planar cell polarity protein 1 (PRICKLE1), protein kinase AMP-activated non-catalytic subunit beta 2 (PRKAB2), PYD and CARD domain containing (PYCARD), RAS p21 protein activator 1 (RASA1), RASD family member 2 (RASD2), RAS guanyl releasing protein 1 (RASGRP1), rhophilin RHO GTPase binding protein 2 (RHPN2), Rab interacting lysosomal protein like 2 (RILPL2), roundabout guidance receptor 1 (ROBO1), RAR related orphan receptor A (RORA), Stromal cell derived factor 4 (SDF4), SERTA domain containing 4 (SERTAD4), shisa family member 5 (SHISA5), signal induced proliferation associated 1 like 2 (SIPA1L2), solute carrier family 22 member 20 (SLC22A20P), solute carrier family 24 member 3 (SLC24A3), solute carrier family 2 member 12 (SLC2A12), solute carrier family 39 member 10 (SLC39A10), solute carrier family 25 member 40 (SLC25A40), solute carrier family 43 member 1 (SLC43A1), solute carrier family 45 member 4 (SLC45A4), solute carrier family 6 member 20 (SLC6A20), spectrin beta, erthrocytic (SPTB), stromal antigen 3-like 3 (STAG3L3), sushi domain containing 3 (SUSD3), TATA-Box binding protein associated factor 10 (TAF10), t-complex-associated testis expressed 3 (TCTE3), transmembrane channel like 7 (TMC7), transmembrane and coiled-coil domain family 2 (TMCC2), transcriptional repressor GATA binding 1 (TRPS1), tubulin tyrosine ligase like 4 (TTLL4), tRNA-yW synthesizing protein 5 (TYW5), ubiquitin conjugating enzyme E2 W (UBE2 W), WD repeat, sterile alpha motif and U-box domain containing 1 (WDSUB1), N-protein N-terminal glutamine amidohydrolase (WDYHV1), N-terminal glutamine amidase 1 (NTAQ1), zinc finger BED-type containing 6 (ZBED6), zinc finger and BTB domain containing 46 (ZBTB46), zinc finger CCCH-type containing 13 (ZC3H13), zinc fingers and homeoboxes 2 (ZHX2), zinc finger protein 217 (ZNF217), zinc finger protein 233 (ZNF233), zinc finger protein 248 (ZNF248), zinc finger protein 469 (ZNF469), and zinc finger protein 785 (ZNF785); and
b) instructions for use.

22. The kit of claim 21, wherein the isolated set of probes capable of detecting a panel of biomarkers comprises at least three, at least four, at least five, at least six, at least seven at least eight, at least nine, or ten biomarkers selected from a biomarker signature as follows:
a) DGKH, GADD45B, KLF7, LYST, NR6A1, PYCARD, ROBO1, SLC22A20P, SLC24A3, and SLC45A4;
b) DGKH, DIP2B, EMP1, GADD45B, MT2A, NOTCH2, NR6A1, RORA, SLC22A20P, and SL24A3;
c) GADD45B, LYST, NOXA1, NR6A1, PYCARD, SLC22A20P, SLC24A3, and NTAQ1;
d) DGKH, KLF7, LYST, NR6A1, ROBO1, SLC24A3, and SLC6A20;
e) DGKH, EMP1, GADD45B, LYST, SLC22A20P, SLC24A3, and SLC6A20;
f) CUEDC1, DGKH, EMP1, LYST, NOXA1, SLC22A20P, and SLC6A20;
g) DGKH, KLF7, LYST, NR6A1, PRICKLE1, ROBO1, and SLC24A3;
h) DCLK2, GADD45B, LYST, NR6A1, SLC22A20P, SLC24A3, and NTAQ1;
i) GADD45B, KLF7, LYST, NR6A1, ROBO1, SLC22A20P, and SLC6A20;
j) DCLK2, GADD45B, LYST, NR6A1, SLC22A20P, and NTAQ1;
k) C12orf65, GADD45B, LRBA, LYST, PEX1, PRKAB2, and TYW5;
l) LYST, PEX1, ODAD1, DEPDC7, MICALL2, SLC43A1, and SLC6A20;
m) C12orf65, GADD45B, LYST, PEX1, RASA1, SLC45A4, and NTAQ1;
n) TYW5, DEPDC7, SLC43A1, CGREF1, MICB, HRAS, and MT2A;
o) LRBA, LYST, PEX1, DEPDC7, SLC43A1, MICB, and C2orf49;
p) GADD45B, PEX1, DEPDC7, SLC43A1, LSM11, and PJA1;
q) DEPDC7, SLC43A1, MICB, C2orf49, HRAS, KCNC3, and MT2A;
r) LYST, DEPDC7, SLC43A1, SLC6A20, MICB, and HRAS;
s) PEX1, MICALL2, SLC43A1, RASA1, KCNC3, and LRIT3;
t) GADD45B, SLC43A1, SLC45A4, KCNC3, SHISA5, and SLC25A40;
u) GADD45B, H2AF, PTPRA, RILPL2, RORA, ZC3H13, and ZHX2; or
v) CLDN4, ERH, GADD45B, GYPC, H2AFX, MT2A, NDUFV2, SDF4, and UBE2 W.

23. The kit of claim 21, wherein the probe is selected from the group consisting of an aptamer, an antibody, an affibody, a peptide, a protein, an organic molecule, and a nucleic acid.
